(19) United States
(12) Patent Application Publication
(10) Pub. No.: US 2025/0152413 A1
(43) Pub. Date: May 15, 2025

(54) SYSTEMS, DEVICES, AND METHODS FOR NEEDLE-LESS OCULAR INJECTIONS

(71) Applicant: Clearside Biomedical, Inc., Alpharetta, GA (US)

(72) Inventors: Rafael Victor ANDINO, Grayson, GA (US); Chen-Rei WAN, Smyrna, GA (US); Trent KAHUTE, Dunwoody, GA (US); Bradford A. RANGE, Dacula, GA (US); Clay G. PRICKETT, Atlanta, GA (US)

(21) Appl. No.: 19/020,585

(22) Filed: Jan. 14, 2025

Related U.S. Application Data
(63) Continuation of application No. PCT/US2023/066324, filed on Apr. 27, 2023.
(60) Provisional application No. 63/389,663, filed on Jul. 15, 2022.

(51) Int. Cl. A61F 9/00; A61F 9/008; A61M 37/00; A61N 1/30

(52) U.S. Cl. CPC A61F 9/0008; A61F 9/00825; A61M 37/0092; A61N 1/303; A61F 2009/00844; A61F 2009/00865; A61M 2037/0007

(57) ABSTRACT

Embodiments described herein relate to systems and methods of delivering medicinal components to target regions of an eye of a subject and/or patient. In some aspects, an apparatus includes a housing; a medicament container configured to accommodate a medicinal component; a sensor configured to measure a physical property of a subject; and an injector coupled to the medicament container and configured to produce a stream of the medicinal component directed towards a region of the eye of the subject based on the measured physical property.

### CROSS-REFERENCES TO RELATED APPLICATION

The present application is a continuation of International Application No. PCT/US2023/066324, filed Apr. 27, 2023, which claims priority to and benefit of U.S. provisional application No. 63/389,663, filed Jul. 15, 2022, entitled Systems, Devices, and Methods for Needle-Less Ocular Injections, which are incorporated by reference in their entireties.

### TECHNICAL FIELD

The present disclosure relates to systems, devices, and methods of drug delivery, and more specifically, to delivery of drugs (e.g., medicinal components) to specific portions and/or regions of a human eye without using a piercing member such as a needle, cannula, and/or catheter (also referred to herein as using a needle-less device or method).

### BACKGROUND

Embodiments described herein relate to systems, devices, and methods for delivery of medicinal components to an eye of (e.g., a human) subject and/or patient. The systems, devices, and methods disclosed herein can be used in non-surgical and/or minimally invasive procedures (e.g., in an office setting, without external imaging, etc.), as well as in a surgical and/or operating room environment. Common methods for delivering medicinal components to the eye of a subject and/or patient include retinal injections. Retinal injections require the use of a piercing member such as a needle that penetrates the different layers of the eye to reach the target region where the medicinal component is deposited. Limitations and/or shortcomings of retinal injections include their invasive nature which elicits pain and thus have lower acceptance and/or compliance by subjects and/or patients (e.g., needle phobia), and/or require more complicated and timely procedures and environments, such as, for example, a surgical room with imaging modalities). Consequently, there is a need to develop methods that can safely and effectively deliver one or more medicinal component to specific regions and/or portions of the eye without using piercing members such as needles, cannulas, and/or catheters.

### SUMMARY

Systems, devices and methods for delivering medicinal components to an eye of a human subject and/or patient without using a piercing member such as a needle, cannula, stylet, and/or catheter (e.g., needle-less medicament delivery) are described herein. In some embodiments, an apparatus includes a housing; a medicament container configured to accommodate a medicinal component; a sensor configured to measure a physical property of a subject; and an injector coupled to the medicament container and configured to produce a stream of the medicinal component directed towards a region of the eye of the subject based on the measured physical property.

### BRIEF DESCRIPTION OF THE DRAWINGS

FIG. 1 is a cross-sectional view of an illustration of a human eye.

FIG. 2 is a cross-sectional view of portion of the human eye of FIG. 1, taken along the line 2-2.

FIGS. 3, 4A, and 4B are cross-sectional views of a portion of the human eye of FIG. 1 taken along the line 3-3, illustrating the suprachoroidal space without the presence of a fluid, the suprachoroidal space with the presence of a fluid, and the retina with the presence of fluid, respectively.

FIG. 5 is a schematic illustration of a medicament delivery device, according to an embodiment.

FIG. 6A is a schematic illustration of a medicament delivery device, according to an embodiment.

FIG. 6B is a schematic illustration of a plot relating the pressure of the gases contained in a high-pressure chamber required to direct a stream of medicinal components to a target region of the sclera of an eye of a subject, as a function of the measured thickness of the sclera tissue layer.

FIG. 7 is a schematic illustration of a medicament delivery device, according to an embodiment.

FIG. 8 is a schematic illustration of a medicament delivery device, according to an embodiment.

FIG. 9A shows a schematic illustration of the medicament delivery device including a tissue manipulation component configured to increase the permeability of medicinal components being delivered to the suprachoroidal space of a human eye

FIG. 9B shows a cross sectional schematic illustration of the medicament delivery device shown in FIG. 9A being used to deliver a medicament on a human eye.

### DETAILED DESCRIPTION

Syringes and hypodermic needles are among the most common devices sued to administer medical components (e.g., a drug) to an eye of a subject and/or patient. The use of syringes as a medium of drug delivery has been common practice and widely accepted in spite of being associated with various drawbacks including: needle-stick injuries, needle phobia (e.g., aversion of subject and/or users to needles), lack of reusability due to potential contamination issues, and cost associated with the manufacture of the needles as well as the training of specialized healthcare workers and medical professionals that can operate the syringes in a safe and reliable manner.

The use of syringes for retinal injections in a human patient pose additional difficulties. For example, human eyes can vary widely in overall size, as well as thickness of various tissue layers. Whether injecting an injectate (e.g., a medicinal component) into a tissue layer proximal to the vitreous body (i.e., pushing the delivery member through a few layers of tissue) or distal to the vitreous body (i.e., pushing the delivery member through the layers of tissue proximal to the vitreous body, the vitreous body, and a few layers of tissue distal to the vitreous body), a large amount of variability exists from patient to patient. For example, sclera thickness in the human eye can range anywhere from about 350 μm to about 700 μm. This variability in tissue layer size, for example, can make it difficult to standardize tooling or methods when precisely injecting into a region of the eye. If using the outermost surface of the eye or a region external to the eye as a reference point, it can be difficult to efficiently and accurately locate a specific tissue or tissue interface in the eye.

Systems and devices described herein address the limitations of existing technologies by providing devices that can deliver controlled amounts of medicinal components directed to specific regions of the eye of a subject and/or patient such as the interface between the sclera tissue layer and the choroid tissue layer (e.g., the suprachoroidal space (SCS)), without using a piercing member such as a needle, cannula, stylet, and/or catheter (e.g., needle-less medicament delivery).

As used herein, the singular forms “a,” “an”, and “the” include plural referents unless the context clearly dictates otherwise. Thus, for example, the term “a member” is intended to mean a single member or a combination of members, “a material” is intended to mean one or more materials, or a combination thereof

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one implementation, to at least one, optionally including more than one, A, with no B present (and optionally including elements other than B); in another implementation, to at least one, optionally including more than one, B, with no A present (and optionally including elements other than A); in yet another implementation, to at least one, optionally including more than one, A, and at least one, optionally including more than one, B (and optionally including other elements); etc.

As used herein, a “set” can refer to multiple features or a singular feature with multiple parts. For example, when referring to set of walls, the set of walls can be considered as one wall with distinct portions, or the set of walls can be considered as multiple walls.

As used herein, the terms “about” and “approximately” generally mean plus or minus 10% of the value stated. For example, about 0.5 would include 0.45 and 0.55, about 10 would include 9 to 11, about 1000 would include 900 to 1100.

As used herein, the terms “medicament container”, and “medicament containment chamber” are used interchangeably to refer to an article configured to contain a volume of a substance, for example, a medicament.

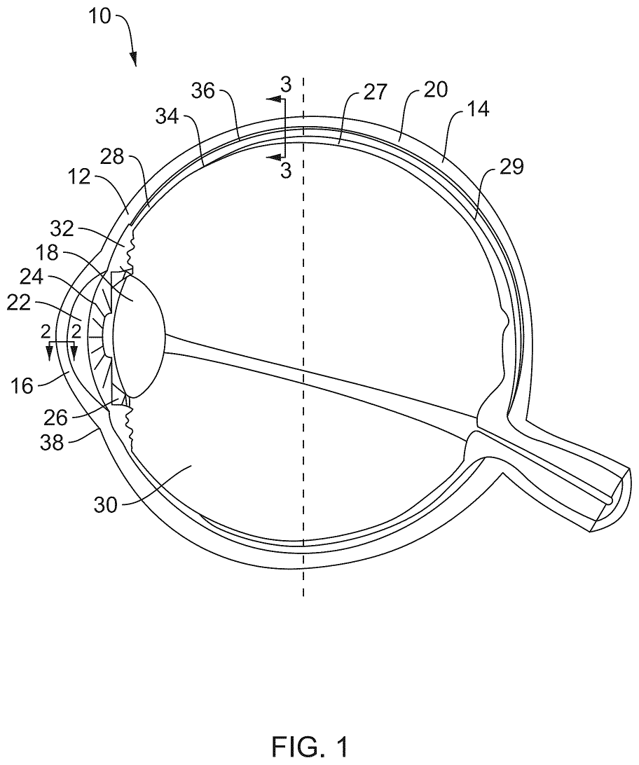

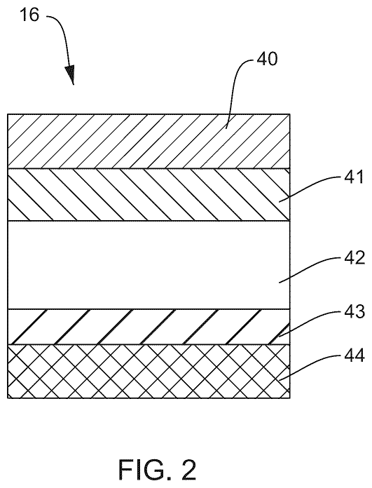

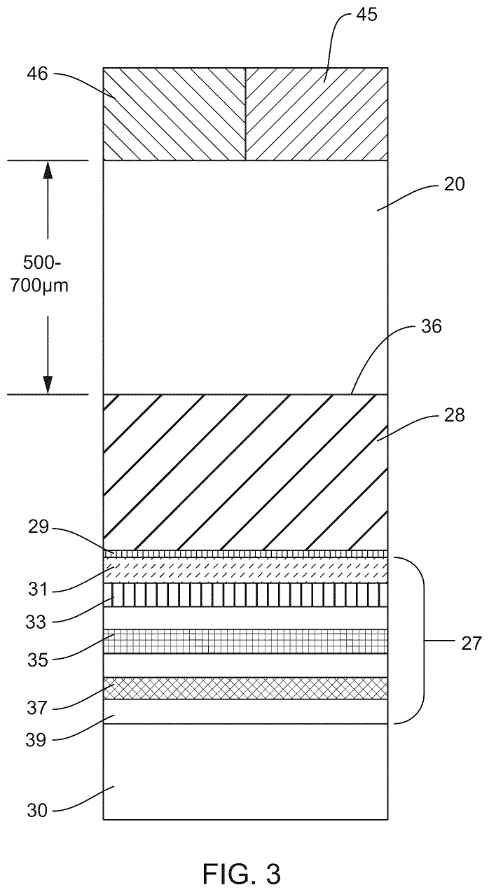

The systems, devices, and methods described herein can be used to deliver substances to various target tissues in the eye without using a piercing member such as a needle, cannula, or catheter. To facilitate the description of the embodiments disclosed herein, FIGS. 1-4 present schematic representations of various views of a human eye 10. While specific regions are identified, those skilled in the art will recognize that the proceeding identified regions do not constitute the entirety of the eye 10, rather the identified regions are presented as a simplified example suitable for the discussion of the embodiments herein. The eye 10 includes both an anterior segment 12 (the portion of the eye in front of and including the lens) and a posterior segment 14 (the portion of the eye behind the lens). The anterior segment 12 is bounded by the cornea 16 and the lens 18, while the posterior segment 14 is bounded by the sclera 20 and the lens 18. The anterior segment 12 is further subdivided into the anterior chamber 22, between the iris 24 and the cornea 16, and the posterior chamber 26, between the lens 18 and the iris 24. The cornea 16 and the sclera 20 collectively form a limbus 38 at the point at which they meet. The exposed portion of the sclera 20 on the anterior segment 12 of the eye is protected by a clear membrane referred to as the conjunctiva 45 (see e.g., FIG. 3). Underlying the sclera 20 is the choroid 28 and the retina 27, collectively referred to as retinachoroidal tissue. The retinal pigment epithelium (RPE) 29 is the most outer layer of the retina 27, depicted adjacent to the choroid 28. A vitreous humour 30 (also referred to as the “vitreous” or “vitreous space”) is disposed between a ciliary body 32 (including a ciliary muscle and a ciliary process) and the retina 27. The anterior portion of the retina 27 forms an ora serrata 34. The loose connective tissue, or potential space, between the choroid 28 and the sclera 20 is referred to as the suprachoroid or suprachoroidal space (SCS). FIG. 2 illustrates the cornea 16, which is composed of the epithelium 40, the Bowman's layer 41, the stroma 42, the Descemet's membrane 43, and the endothelium 44

FIG. 3 illustrates the sclera 20 with surrounding Tenon's Capsule 46 or conjunctiva 45, suprachoroidal space 36, choroid 28, and retina 27, substantially without fluid and/or tissue separation in the suprachoroidal space 36 (i.e., the in this configuration, the space is “potential” suprachoroidal space). As shown in FIG. 3, the sclera 20 has a thickness between about 350 μm and 700 μm. FIG. 3 illustrates sections of the retina 27 including RPE 29, rod and cone outer segments 31, rod and cone nuclei 33, bipolar cells 35, ganglion cells 37, and nerve fibers to the optic nerve 39. FIG. 3 also includes the vitreous humour 30.

FIG. 4A illustrates the sclera 20 with the surrounding Tenon's Capsule 46 or the conjunctiva 45, suprachoroidal space 36, choroid 28, RPE 29, retina 27, with fluid 50 in the SCS 36. FIG. 4B illustrates the sclera 20 with the surrounding Tenon's Capsule 46 or the conjunctiva 45, suprachoroidal space 36, choroid 28, RPE 29, retina 27, with fluid 51 in the retina 27 (between the RPE 29 and the rod and cone outer segments 31)

As used herein, the term “suprachoroidal space,” or SCS which is synonymous with suprachoroid, or suprachoroidia, describes the space (or volume) and/or potential space (or potential volume) in the region of the eye 10 disposed between the sclera 20 and choroid 28. This region primarily is composed of closely packed layers of long pigmented processes derived from each of the two adjacent tissues; however, a space can develop in this region because of fluid or other material buildup in the suprachoroidal space and the adjacent tissues. The suprachoroidal space can be expanded by fluid buildup because of some disease state in the eye or because of some trauma or surgical intervention. In some embodiments, the fluid buildup is intentionally created by the delivery, injection and/or infusion of a drug formulation into the suprachoroid to create and/or expand further the suprachoroidal space 36 (i.e., by disposing a drug formulation therein). This volume may serve as a pathway for uveoscleral outflow (i.e., a natural process of the eye moving fluid from one region of the eye to the other through) and may become a space in instances of choroidal detachment from the sclera.

As used herein, the term “retina” or “retinal space” can include the space (or volume) and/or potential space (or potential volume) between the RPE 29, and (1) the interface between the nerve fibers to the optic nerve 39 and the vitreous body 30, or (2) the photo receptor layer 31.

As used herein, the space between the choroid 28 and the retina 27 can include the space (or volume) and/or potential space (or potential volume) on either side adjacent to the RPE 29 or space within the RPE 29 itself.

The dashed line in FIG. 1 represents the equator of the eye 10. In some embodiments, the devices, systems and methods described herein can facilitate delivering a medicament without the use of a piercing member and/or needle, introducing the medicament through a region of the eye between the equator and the limbus 38 (i.e., in the anterior portion 12 of the eye 10). For example, in some embodiments, the medicant can be delivered through a region and/or portion of the eye between about two millimeters and 10 millimeters (mm) posterior to the limbus 38. In other embodiments, the medicant can be delivered through a region and/or portion of the eye about the equator of the eye 10. In still other embodiments, the medicant can be delivered through a region and/or portion of the eye that is posterior to the equator of the eye 10

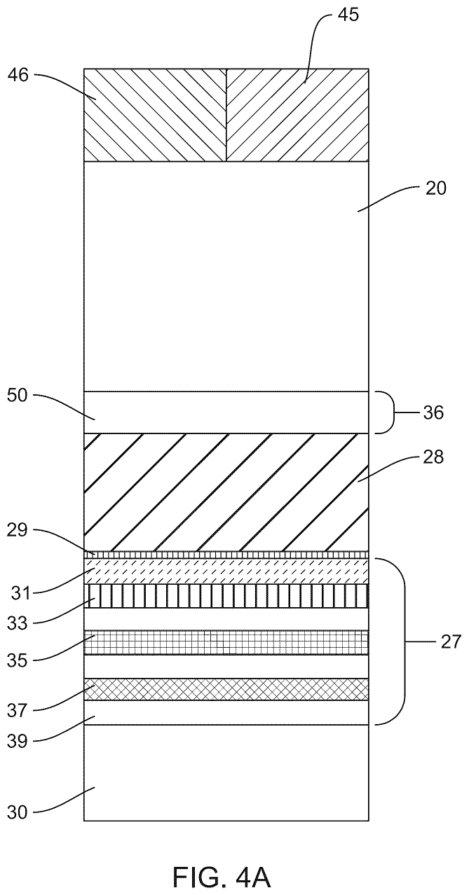

FIG. 5 shows a schematic illustration of a medicament delivery device 100 according to an embodiment. The medicament delivery device 100 (also referred to herein as the “delivery device”) can be configured to delivery one or more medicaments and/or medicinal components to specific portions and/or regions of a human eye without using a piercing member such as a needle, cannula, stylet, and/or catheter (e.g., needle-less medicament delivery). For example, in some implementations the delivery device 100 can be used to deliver medicinal components to a retinal space of the eye or a retinal tissue. In some implementations, the delivery device 100 can used to deliver medicinal components into a region proximal to the retinal space of the eye or retinal tissue (e.g., between the choroid and the retina, or within the choroid adjacent its distal edge such that injectate injected therein could travel towards and into the retina). In some embodiments the medicaments and/or medicinal components can include, for example, an anti-inflammatory compound, a tyrosine kinase inhibitor, an integrin inhibitor, a complement inhibitor, a VEGF inhibitor, a gene therapy, an αvβ3 inhibitor, an αvβ5 inhibitor, an αvβ1 inhibitor, an α4β1 inhibitor, or an α4β7 inhibitor, axitinib, MK-409, and/or aflibercept, as further described herein. The delivery device 100 can include a housing 110, a medicament container 120, an injector 130, one or more sensor(s) 140, and a control unit 150. Optionally, in some embodiments the delivery device 100 can include a tissue manipulation component 160 (also referred to herein as a tissue manipulator), and/or a contact member 170. The housing 110 can be any suitable structure that defines one or more volumes, compartments, chambers, and/or surfaces for accommodating (e.g., housing, containing, supporting, coupling, etc.) one or more components of the delivery device 100. For example, as shown FIG. 5, in some implementations the housing 110 can accommodate the medicament container 120, the injector 130, the sensor(s) 140, the control unit 150, and the optional tissue manipulation component 160. The housing 110 can also provide one or more surfaces that can facilitate coupling the optional contact member 170. The medicament container 120 can be any suitable structure capable of containing and/or storing one or more medicaments. The medicament container 120 can be mechanically and fluidically coupled, at least temporarily, to the injector 130. The injector 130 can be any suitable component configured to transport and/or convey the one or more medicaments from the medicament container 120 to a selected and/or intended region, portion, and/or section of an eye of a subject, patient, and/or user. The sensor(s) 140 can include any type of sensor capable of detecting and/or measuring one or more physiological and/or physical property and/or characteristic of the eye (or a portion thereof) of the subject, patient, and/or user. The sensor(s) 140 can be operatively coupled with the injector 130 and the control unit 150 to measure a property of the eye of the subject and communicate the measured property to the control unit 150. The control unit 150, in response to receiving the measured property from the sensor(s) 140 can adjust and/or control one or more parameters of the injector 130 such that the injector 130 can precisely, and accurately transport and/or convey the one or more medicaments from the medicament container 120 to the intended region, portion, and/or section of the eye of the subject, patient, and/or user.

As described above, the housing 110 can be any suitable structure configured to define one or more volumes, compartments, chambers, and/or surfaces for accommodating and/or coupling the various components of the delivery device 100. In some implementations, the housing 110 can include and/or define a single internal volume and/or chamber in which multiple components such as the medicament container 120, the injector 130, the sensor(s) 140, and/or the control unit 150 can be accommodated. Alternatively, in some implementations the housing 110 can include multiple internal volumes and/or chambers configured to house any suitable combination or distribution of specific components of the delivery device 100. The housing 110 can be configured to provide one or more surfaces that can be used to couple one or more components of the delivery device 100 such as the contact member 170, as well as one or more third-party component and/or device. The housing 110 can be any suitable shape and/or size sufficient to accommodate and/or support the one or more components of the delivery device 100. For example, in some implementations the housing 110 can be an elongate member having a suitable geometrical cross-sectional shape, including, for example, triangular, circular, square, rectangular, hexagonal, polygonal, and/or a combination thereof. In some implementations, the housing 110 can have a shape, surface features, and/or surface materials or finishes that can be configured to increase the ergonomics of the delivery device 100, which can, for example, allow a trained healthcare worker, a medical professional and/or a user to manipulate the delivery device 100 with one hand (i.e., single-handed use).

The housing 110 can be made of any suitable material or materials having sufficient structural strength and rigidity, including, for example, metal, glass, ceramic, and/or polymers. In some implementations, the housing 110 can include multiple portions that can be coupled and/or assembled together to form one or more chambers and/or compartments for receiving the components of the delivery device 100. That is, in some implementations, the housing 110 can be modular. Alternatively, in other implementations, the housing 110 can be made of a monolithic structure.

The medicament container 120 can be any suitable structure capable of containing and/or storing one or more medicinal components to be delivered to an eye of a subject and/or patient. For example, in some implementations the medicament container 120 can include one or more reservoir(s) sized and shaped to contain and/or store (at least temporarily) a volume of a liquid containing the medical component(s). In some implementations, the medicament container 120 can include a reservoir sized and shaped to contain and/or store (at least temporarily) a volume of a solid (e.g., a powder), a gas (or a gas mixture), a dispersion, an emulsion, and or the like containing the medicinal component(s). In some implementations the medicament container 120 can include and/or define multiple chambers and/or compartments, with each chamber and/or compartment being seized and shaped to contain and/or store a predetermined volume, amount and/or type of medicinal component(s). For example, in some implementations the medicament container 120 can include a first chamber sized and shaped to accommodate a first medicinal component(s). The medicament container 120 can include a second chamber sized and shaped to accommodate a second medicinal component(s). The first and/or the second component(s) can be liquids, solids, gases, emulsions, dispersions, or any suitable combination thereof. In some implementations the medicament container 120 can be disposed entirely inside the housing 110. In some implementations the medicament container 120 can be external to the housing (e.g., coupled to and/or attached to the housing externally). In some implementations, a first portion of the medicament container 120 can be disposed inside the housing 110, while and a second portion of the housing can be disposed and/or coupled to the exterior of the housing 110. In some implementations the housing 110 can act and/or serve as the medicament container 120.

As described above, the medicament container 120 can include one or more chambers and/or compartments defining a volume suitable to accommodate one or more medicinal component(s). In some implementations the medicament container 120 can define a volume of at least about 0.1 mL, at least about 0.2 mL, at least about 0.3 mL, at least about 0.4 mL, at least about 0.5 mL, at least about 0.6 mL, at least about 0.7 mL, at least about 0.8 mL, at least about 0.9 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL, at least about 20 mL, at least about 30 mL, at least about 40 mL, at least about 50 mL, at least about 60 mL, at least about 70 mL, at least about 80 mL, at least about 90 mL, at least about 100 mL, at least about 200 mL, at least about 300 mL, or at least about 400 mL. In some embodiments, the medicament container 120 can define a volume of no more than about 500 mL, no more than about 400 mL, no more than about 300 mL, no more than about 200 mL, no more than about 100 mL, no more than about 90 mL, no more than about 80 mL, no more than about 70 mL, no more than about 60 mL, no more than about 80 mL, no more than about 70 mL, no more than about 60 mL, no more than about 50 mL, no more than about 40 mL, no more than about 90 mL, no more than about 80 mL, no more than about 70 mL, no more than about 60 mL, no more than about 50 mL, no more than about 40 mL, no more than about 30 mL, no more than about 20 mL, no more than about 10 mL, no more than about 9 mL, no more than about 8 mL, no more than about 7 mL, no more than about 6 mL, no more than about 5 mL, no more than about 4 mL, no more than about 3 mL, no more than about 2 mL, no more than about 1 mL, no more than about 0.9 mL, no more than about 0.8 mL, no more than about 0.7 mL, no more than about 0.6 mL, no more than about 0.5 mL, no more than about 0.4 mL, no more than about 0.3 mL, or no more than about 0.2 mL.

Combinations of the above-referenced volumes of the medicament container 120 are also possible (e.g., at least about 1 mL and no more than about 500 mL or at least about 50 mL and no more than about 100 mL), inclusive of all values and ranges therebetween. In some embodiments, the medicament container 120 can define a volume of about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 7 mL, about 8 mL, about 9 mL, about 10 mL, about 20 mL, about 30 mL, about 40 mL, about 50 mL, about 60 mL, about 70 mL, about 80 mL, about 90 mL, about 100 mL, about 200 mL, about 300 mL, about 400 mL, or about 500 mL.

FIG. 5 shows the medicament container 120 can be coupled to the injector 130 to provide access to the medicinal component(s) contained and/or stored in the medicament container 120 such that the medicinal component(s) can be delivered (via the injector 130) to one or more regions and/or portions of an eye of a subject and/or patient. The medicament container 120 can include an ejection port, opening, orifice, aperture, and/or passage (not shown) that facilitates evacuating the medicament(s) contained and/or stored in the medicament container 120. In the implementations in which the medicament container 120 includes multiple chambers and/or compartments, the medicament container 120 can also include multiple ejection ports, openings, orifices, apertures, and/or passages to facilitate evacuating the medicament component(s) stored inside those chambers and/or compartments. The ejection port(s), opening(s), orifice(s), aperture(s), and/or passage(s) can be disposed and/or located on a portion or region of the medicament compartment 120 such that the injector 130 can produce a flow of the medicinal component(s) out of the medicament container 120 and into one or more nozzle(s) of the injector 130 (not shown), as further described herein.

The injector 130 can be any suitable component coupled to the medicament container 120 and configured to (1) produce a flow of medicinal component(s) stored and/or contained in the medicament compartment 120, (2) direct and/or guide the produced flow of medicinal component(s) to a nozzle (not shown), and (3) generate, with the aid of the nozzle, a stream having one or more predetermined characteristics including: a width, a cross sectional area, a cross sectional shape, a velocity of the medicinal component(s) (e.g., particle velocity), and/or a specific trajectory that transports the medicinal component(s) to a target region and/or portion of an eye of a subject and/or patient. In some implementations the nozzle can be a stand-alone component and/or formed separately and that can be removably coupled to the injector 130 (e.g., an exchangeable nozzle) while in other implementations the nozzle can be integrated, monolithically formed with, and/or effectively permanently incorporated into the injector 130. In some implementations the nozzle can include one or more holes (e.g., a fine hole), openings, apertures, slots or the like disposed on a substrate material (e.g., metal, ceramic, glass, polymer, etc.). The shape, size, and distribution or position of the fine holes can be configured such that the flow of medicinal component(s) through the fine holes generates the stream of the medicinal component(s) having the predetermined characteristics described above. In some implementations, the nozzle can be a component that defines a pathway for the medicinal component(s) to flow. This pathway can be governed by an electromagnetic field configured to interact with electrically charged species (e.g., anions and/or cations) of the medicinal component(s) and accelerate them to generate the stream of predetermined characteristics. In such implementations, one or more characteristics of the electromagnetic field (e.g., magnitude or strength, direction, on-off frequency, etc.) can be controlled by the control unit 150 to adjust and/or change the predetermined characteristics of the stream of medicinal component(s).

The injector 130 can produce a flow of the medicinal component(s) stored and/or contained in the medicament compartment 120 directed towards the nozzle. The injector 130 can produce the flow of medicinal component(s) by increasing the kinetic energy of the medicinal component(s) via mechanical, electrical, and/or a magnetic forces and/or interactions. For example, in some implementations the injector 130 can generate the flow of medicinal component(s) by means of mechanical forces and/or interactions. In such implementations, the injector 130 can be coupled to the medicament container 120 such that the injector 130 (or a portion thereof) can exert forces on the medicinal component(s) using a movable wall and/or piston to transfer kinetic energy to the medicinal component(s) and produce a flow of medicinal components out of the medicament container 120. For example, in some implementations the injector 130 can include a positive displacement pump (not shown) such as a piston pump or the like, fluidically coupled to the medicament container 120. The piston pump can include a piston disposed inside a cylinder (e.g., a piston cup), two or more valves (e.g., a suction valve and a discharge valves), and an oscillating mechanism. The oscillating mechanism can be configured to move the piston in an up-stroke and a down-stroke movement. In the up-stroke movement, the suction valve can be opened, and the piston can be moved (e.g., upstroke movement) to generate a pressure differential within the medicament container that causes a flow of medicinal component(s) filling the piston cup, a pump chamber, and/or a pump reservoir. The oscillating mechanism can then be configured to move the piston in the down-stroke movement. In the down-stroke movement, the suction valve can be closed while the discharge valve is opened. The piston can be moved (e.g., down-stroke movement) to push out the medicinal component(s) contained in the piston cup, pump chamber, and/or pump reservoir, generating the flow of medicinal component(s) directed towards the nozzle.

In some implementations, the injector 130 can include a high-pressure chamber and a pressurized fluid (not shown). The high-pressure chamber can be mechanically coupled to the medicament container 120. The high-pressure gas chamber can include and/or contain one or more pressurized fluids such as a compressed gas (not shown) disposed inside the high-pressure chamber, and a movable component (not shown) such as a wall and/or a piston coupled to the medicament component 120. The compressed gases can be used to move the movable component of the injector 130 to exert a force on the medicament container 120 (or a portion thereof). In some implementations the movable wall and/or a piston of the high-pressure gas chamber can be mechanically coupled to a movable wall and/or piston of the medicament container 120 disposed in direct contact with the medicinal component(s). Moreover, in some implementations the movable wall and/or a piston of the high-pressure gas chamber can also serve as the movable wall and/or piston of the medicament container 120. The forces exerted by the movable component of the injector 130 on the medicament container 120 can increase kinetic energy to the medicinal component(s) (e.g., accelerate the medicinal component(s) stored inside the medicament container 120). The high-pressure chamber can assume a first state and/or configuration (also referred to herein as a “compressed” configuration) in which the compressed gases are kept inside the high-pressure chamber occupying a first volume at a first pressure. The high-pressure chamber can be transitioned from the first state and/or configuration to a second state and/or configuration (also referred to herein as an “expanded” configuration). In the second state and/or configuration the compressed gases are allowed to expand and move the movable component (e.g., a movable wall and/or a piston) until the compressed gas is contained inside the high pressure chamber within a second volume and at a second pressure, the second volume being larger than the first volume, and the second pressure being lower than the first pressure The movement of the movable wall and/or piston can effectively apply a force on the medicament container 120 (or a portion thereof) such that the medicinal component(s) increase their kinetic energy and a flow of the medicinal component(s) is directed out of the medicament container 120 towards a nozzle of the injector 130. As discussed above, the nozzle of the injector 130 can receive the flow of medicinal component(s) and generate a stream that can be directed to the intended target region and/or portion of the eye of the subject and/or patient.

In some implementations, the injector 130 can include a high-pressure chamber containing the compressed gases, and an outlet port and/or opening, that can be operated to fluidically couple the high-pressure chamber and the medicament container 120. In such embodiments, the high-pressure chamber can assume a first state and/or configuration (also referred to herein as a “compressed” configuration) in which the compressed gases are kept inside the high-pressure chamber at a first pressure. The high-pressure chamber can be transitioned from the first state and/or configuration to a second state and/or configuration (also referred to herein as an “expanded configuration). In the second state and/or configuration at least a portion of the gases are directed, transferred and/or flown through the outlet port into the medicament container 120. Exposure of the medicinal component(s) to the compressed gases in the medicament container 120 can cause dispersion of the medicinal components in the gas atomizing the medicinal component(s). The gas including the atomized medicinal component(s) can be directed to the nozzle of the injector 130 to generate a stream that can be guided to the intended target region and/or portion of the eye of the subject and/or patient.

In some implementations, the injector 130 can include a Lorentz force motor (not shown) coupled to the medicament container 120. The Lorentz force motor can be configured to engage a movable component such as a wall and/or a piston to move and/or accelerate such component using a magnetic force and/or interaction. The Lorentz force motor can include a magnet and a coil (e.g., conductor) to convert electrical energy to mechanical energy according to the Lorentz force principle, which states that a current-carrying conductor located within a magnetic field can generate a force (Lorentz or electromagnetic force). The magnitude of the Lorentz force is proportional to the electrical current passing through the conductor and the magnetic flux produced by the magnet, producing linear force or torque output, and high acceleration or frequency actuation. In some implementations the Lorentz force motor can include a permanent magnet and one or more coils disposed around the magnet. The coils can be disposed around the magnet according to any suitable arrangement and/or layout. For example, in some implementations the coils can be disposed around the magnet according to a toroidal arrangement. In other implementations, the coils can be disposed around the magnet according to a circular, elliptical, and/or other arrangement and/or layout. The coil can be made of an electrically conductive material suitable for passing an electric current. In some implementations the coil can be configured to pass a direct current (e.g., a DC current). In some implementations the coil can be configured to pass an alternating current (e.g., an AC current). Moreover, in some implementations, the coil can be configured to pass either a DC current or an AC current. That is, in some instances, the coil can be configured and/or used to pass a DC current, and in other instances the same coil can be used to pass an AC current.

The electrical current passed thought the coils can induce a magnetic field that interacts with the magnetic field produced magnet. The interaction and/or coupling of the magnetic fields (e.g., the permanent magnetic field of the magnet and the induced magnetic field generated by passing an electrical current through the coils) can generate a magnetic force that acts on the magnet and causes movement of the magnet along a predetermined axis (e.g., the direction of the predetermined axis can be determined based on the of direction of the magnetic field generated by the current passing through the coil, and the direction of the magnetic field of the magnet). The magnet can be mechanically coupled to a movable wall and/or piston of the injector 130 such that the movement of the magnet under the effect of the magnetic force can transfer the movement to the movable wall and/or piston. The movable wall and/or piston of the injector 130 can also be mechanically coupled to at least a portion of the medicament container 120. In that way, the movement of the movable wall and/or piston of the injector 130 caused by the magnetic field in the injector 130 can exert a force on the medicament container 120. The forces exerted by the movable component of the injector 130 on the medicament container 120 can increase kinetic energy to the medicinal component(s) (e.g., accelerate the medicinal component(s) stored inside the medicament container 120). The movement of the movable wall and/or piston of the medicament container can generate a flow of the medical component(s) out of the medicament container 120 via the one or more ejection port(s), opening(s), orifice(s), aperture(s), and/or passage(s) of the medicament container 120. The flow of the medicinal component(s) can be directed towards the nozzle of the injector 130 to generate a stream that can be guided to the intended target region and/or portion of the eye of the subject and/or patient. As described above, in some implementations the nozzle can be coupled to the injector 130 and can be configured to fine tune the width of the stream, the velocity of the medicinal component(s) in the stream, and/or the trajectory of the stream towards the eye of the subject and/or patient.

In some implementations, the injector can include a DC motor, AC motor, synchronous motor, induction motor, and/or the like coupled to the medicament container 120. The motor can include a stator, a rotor, an axle, and/or a commutator configured to move a movable component such as a wall and/or a piston of the injector 130. For example, in some implementations the motor can be a DC motor comprising a stator, a rotor, a commutator and an axle. The stator can be a permanent magnet that produces a static magnetic field. The rotor can include multiple coils of wire with a current running through to generate an electromagnetic field aligned with the center of the coils. The coils of wire can be disposed within the north pole and the south pole of the permanent magnet and can be coupled to the commutator. The commutator can allow each coil to be energized in a predetermined sequence (e.g., passing a current through eh coil). The commutator can turn on and off the coils in sequence to create a rotating magnetic field that interacts with the field of the permanent magnet in the stator, creating torque, and causing the rotor to rotate. The rotor can be coupled to an axle to transfer the movement generated by the DC motor. The movement of the axle can be converted to a linear displacement movement to move a wall and/or piston of the injector 130. The movable wall and/or piston can be mechanically coupled to at least a portion of the medicament container 120. In that way, the movement of the movable wall and/or piston of the injector 130 caused by the DC motor can exert a force on the medicament container 120. The forces exerted by the movable component of the injector 130 on the medicament container 120 can increase kinetic energy to the medicinal component(s) (e.g., accelerate the medicinal component(s) stored inside the medicament container 120). The movement of the movable wall and/or piston of the medicament container can generate a flow of the medical component(s) out of the medicament container 120 via the one or more ejection port(s), opening(s), orifice(s), aperture(s), and/or passage(s) of the medicament container 120. The flow of the medicinal component(s) can be directed towards the nozzle of the injector 130 to generate a stream that can be guided to the intended target region and/or portion of the eye of the subject and/or patient. As described above, in some implementations the nozzle can be coupled to the injector 130 and can be configured to fine tune the width of the stream, the velocity of the medicinal component(s) in the stream, and/or the trajectory of the stream towards the eye of the subject and/or patient.

In some embodiments the injector 130 can be configured to directly interact and/or engage the medicinal component(s) stored inside the medicament container 120 via magnetic field and/or electric field interactions, to move and/or accelerate the medicinal component(s) and generate a high velocity stream of the medicinal component(s) that can be directed towards the target region and/or portion of the eye of the subject and/or patient. For example, in some implementations the injector 130 can include a magnetic levitation device. The magnetic levitation device can include a power source (not shown), and one or more magnetized coils and/or magnetic loops (not shown) coupled to the power source and disposed on the medicament container 120 forming a linear track and/or guideway along the direction of the nozzle. The magnetized coils can be made of a conductive material such as aluminum, copper, or the like. The power source can pass an electric current through the magnetized coils to create a unique system of magnetic fields capable of interacting directly with medicinal component(s) stored inside the medicament container that have and/or include chemical species with an electrostatic charge (e.g., an ionic species), as further described herein.

The magnetized coils can produce magnetic fields that oppose and/or attract the medicinal component(s) having and/or bearing an electrostatic charge to guide them along the track and/or guideway in the direction of the nozzle. For example, in some implementations, the magnetized coils can be disposed such that the magnetized coils generate an alternating magnetic field. That is a first magnetized coil can be disposed on the track and/or guideway and be configured to produce a first magnetic field that attracts the electrostatic charge of the medicinal component(s). A second magnetized coil can be disposed on the track and/or guideway adjacent to the first magnetized coil, and at a predetermined distance. The second magnetized coil can be configured to produce a second magnetic field that opposes and/or repels the electrostatic charge of the medicinal component(s). The track and/or guideway can include an alternating sequence of the first and the second magnetized coil to generate an alternating magnetic field along the track and/or guideway. The alternating magnetic field can accelerate the medicinal component(s) and increase their kinetic energy propelling them along the direction of the track and/or guideway. The accelerated medicinal component(s) can then be directed out of the medicinal container 120 via the one or more ejection port(s), opening(s), orifice(s), aperture(s), and/or passage(s) of the medicament container 120, producing a flow of the medicinal component(s). The flow of the medicinal component(s) can be directed towards the nozzle to generate a stream that can be guided to the intended target region and/or portion of the eye of the subject and/or patient. In some instances, the flow of medicinal component(s) produced by the injector 130 can be controlled and/or fined tuned by manipulating one or more parameters and/or variables of the injector 130 including, for example, the voltage supplied by the power source, and/or the current passed through the magnetized coils. In some implementations the medicinal component(s) having an electrostatic charge can include a scaffold that contains medicinal component(s) as well as ferromagnetic materials infiltrated. In some implementations, the medicinal component(s) can be disposed, embedded, and/or bound to a conductive polymer such as poly (dimethyl aminopropyl acrylamide (PDMAPAA), hydrogels, and/or the like. In some implementations, the medicinal component(s) can be coupled to, and/or bound to magnetic nanoparticles (MNPs). In some implementations the MNPs can be coupled to the medicinal components via covalent bonds and/or covalent interactions. In some implementations the MNPs can be coupled to the medicinal components via noncovalent interactions. For example, in some implementations the MNPs can be coupled to the medicinal component(s) via ionic interactions, hydrogen bonding, Van der Waals forces, x-effects, hydrophilic/hydrophobic effect, or the like.

FIG. 5 shows the injector 130 can be coupled to an optional control unit 150. In some implementations the injector 130 can be electrically and/or operatively coupled to the control unit 150 to activate, and/or control the operation of the injector 130. More specifically, in some implementations the control unit 150 can be configured to receive one or more signals from the sensor(s) 140 associated with a measured characteristic and/or property of the eye of the subject and/or patient, as further described herein. In response to receiving the signals, the control unit 150 can adjust one or more operating conditions of the injector 130 such that the injector 130 can transfer sufficient kinetic energy to the medicinal component(s) to direct the medicinal component(s) to the intended target region and/or portion of the eye of the subject and/or patient via the nozzle. For example, in some implementations the control unit 150 can be operably coupled to the injector 130 to adjust the operation of the high-pressure chamber of the injector 130. In such implementations the control unit 150 can be configured to control and/or define one or more operating conditions of the high pressure-chamber including, but not limited to, the pressure of the compressed gases contained in the high-pressure chamber of the injector 130, the transitioning between the first and the second configuration and/or state of the high-pressure chamber, and/or the magnitudes of the first pressure, the second pressure, the first volume and/or the second volume of the high-pressure chamber.

In use, the control unit 150 can receive one or more signals from the sensor(s) 140, with the signals being associated with one or more characteristics, parameters, and/or properties of one or more layers of the eye, such as, for example, the sclera, choroid, and/or retina tissue layers of the eye of the subject and/or patient (either each layer by itself, or in combination), as measured by the sensor(s) 140. The control unit 150 can include a processor (not shown) and a transducer (not shown) configured to receive and analyze the signals received from the sensor(s) 140 (e.g., electrical, optical, acoustic, thermal, and/or mechanical signals) and determine, based on the signals received, the one or more characteristics, parameters, and/or properties of the eye of the subject and/or patient. The processor can be further configured to adjust one or more operating conditions of the high-pressure chamber described above, based on the determined characteristics, parameters and/or properties of the eye of the subject and/or patient. In some implementations the one or more characteristics, parameters, and/or properties of the eye of the subject and/or patient can include a thickness of a sclera layer, a thickness of a choroid layer, a thickness of a retina (e.g., a retinachoroidal tissue), a permeability of the sclera layer, a permeability of the choroid layer, a permeability of a retina, a distance and/or depth between the outer surface of the eye of the subject and/or patient and an interface between the sclera layer and the choroid layer, a density of the sclera, the choroid, the retina and/or other tissue layers, an electrical and/or an ionic impedance of the sclera, the choroid, the retina and/or other tissue layers, an optical impedance, reflectivity, and/or absorbance of the sclera, the choroid, the retina and/or other tissue layers, an acoustic impedance, reflectivity and/or an absorbance of the sclera, the choroid, the retina and/or other tissue layers, a thermal impedance, reflectivity, and/or absorbance of the sclera, the choroid, the retina and/or other tissue layers, and/or a distance and/or depth between the outer surface of the eye of the subject and/or patient and an interface between the choroid layer and the retina. In some implementations the control unit 150 can adjust the operating conditions of the high-pressure chamber based on the determined characteristics and/or properties of the eye of the subject and/or patient as well as on the type of nozzle of the injector 130 (e.g., nozzle size, shape, and other technical specifications). For example, as described above, the control unit 150 can adjust the pressure of the compressed gases contained in the high-pressure chamber of the injector 130, the timing of the transitioning between the first configuration and/or state and the second configuration and/or state of the high-pressure chamber, and/or the magnitudes of the first pressure, the second pressure, the first volume and/or the second volume of the high-pressure chamber. The control unit 150 can interact with the injector 130 to adjust the operating conditions of the high-pressure chamber and then cause and/or trigger the transition of the high-pressure chamber form the first state to the second state to produce the flow of the medicinal component(s). The flow of the medicinal component(s) can be directed to the nozzle of the injector 130 to generate a stream having a predetermined width of the stream, velocity of the medicinal component(s) in the stream, and/or trajectory of the stream towards the eye of the subject and/or patient, as described above.

In some implementations, the control unit can be operably coupled to the injector 130 to adjust the operation of the piston pump of the injector 130. In such implementations, the control unit 150 can be configured to control and/or define one or more operating conditions of the piston pump including, but not limited to, the timing of the opening and closing of the suction and/or the discharge valves, the travel length of the piston (e.g., the distance that the piston can be moved in the up-stroke and/or down-stroke movement), the speed with which the piston is moved, the interior volume of the piston cup, pump chamber, and/or a pump reservoir, the frequency of the up-stroke and/or down-stroke movement, and/or the pressure and/or the velocity of the flow of medicinal component(s) produced by the piston pump.

In use, the control unit 150 can receive one or more signals from the sensor(s) 140, with the signals being associated with one or more characteristics, parameters, and/or properties of the sclera, choroid, and/or retina tissue layers of the eye of the subject and/or patient (either each layer by itself, or in combination), as measured by the sensor(s) 140. The control unit 150 can include a processor (not shown) and a transducer (not shown) configured to receive and analyze the signals received from the sensor(s) 140 (e.g., electrical, optical, acoustic, thermal, and/or mechanical signals) and determine, based on the signals received, the one or more characteristics, parameters, and/or properties of the eye of the subject and/or patient. The processor can be further configured to adjust one or more operating conditions of the piston pump described above, based on the determined characteristics, parameters, and/or properties of the eye of the subject and/or patient. In some implementations the one or more characteristics, parameters, and/or properties of the eye of the subject and/or patient can include a thickness of a sclera layer, a thickness of a choroid layer, a thickness of a retina (e.g., a retinachoroidal tissue), a permeability of the sclera layer, a permeability of the choroid layer, a permeability of a retina, a distance and/or depth between the outer surface of the eye of the subject and/or patient and an interface between the sclera layer and the choroid layer, a density of the sclera, the choroid, the retina and/or other tissue layers, an electrical and/or an ionic impedance of the sclera, the choroid, the retina and/or other tissue layers, an optical impedance, reflectivity, and/or absorbance of the sclera, the choroid, the retina and/or other tissue layers, an acoustic impedance, reflectivity and/or an absorbance of the sclera, the choroid, the retina and/or other tissue layers, a thermal impedance, reflectivity, and/or absorbance of the sclera, the choroid, the retina and/or other tissue layers, and/or a distance and/or depth between the outer surface of the eye of the subject and/or patient and an interface between the choroid layer and the retina. In some implementations the control unit 150 can adjust the operating conditions of the piston pump based on the determined characteristics, parameters, and/or properties of the eye of the subject and/or patient, as well as on the type of nozzle of the injector 130 (e.g., nozzle size, shape, and other technical specifications). For example, as described above, the control unit 150 can adjust the timing of the opening and closing of the suction and/or the discharge valves, the travel length of the piston, the speed with which the piston is moved, the interior volume of the piston cup, pump chamber, and/or a pump reservoir, the frequency of the up-stroke and/or down-stroke movement, and/or the pressure and/or the velocity of the flow of medicinal component(s) produced by the piston pump. The control unit 150 can interact with the injector 130 to adjust the operating conditions of the piston pump and then produce the flow of the medicinal component(s). The flow of the medicinal component(s) can be directed to the nozzle of the injector 130 to generate a stream having a predetermined width of the stream, velocity of the medicinal component(s) in the stream, and/or trajectory of the stream towards the eye of the subject and/or patient, as described above.

In some implementations, the control unit 150 can be operably coupled to the injector 130 to adjust and/or control the operation of the Lorentz force motor of the injector 130. In such implementations the control unit 150 can be configured to control and/or define one or more operating conditions of the Lorentz force motor including, but not limited to, the type of current to be passed through the coil of the Lorentz force motor (e.g., an AC current or a DC current), the magnitude of the current passed on the coil, the frequency of an AC current passed through the coils, and/or the length that along an axis that the magnet is allowed to move in response to the magnetic force generated by the Lorentz force motor.

In use, the control unit 150 can receive one or more signals from the sensor(s) 140, with the signals being associated with one or more characteristics, parameters, and/or properties of the choroid, and/or retina tissue layers of the eye of the subject and/or patient (either each layer by itself, or in combination), as measured by the sensor(s) 140. The control unit 150 can include a processor (not shown) and a transducer (not shown) configured to receive and analyze the signals received from the sensor(s) 140 (e.g., electrical, optical, acoustic, thermal, and/or mechanical signals) and determine, based on the signals received, the one or more characteristics, parameters, and/or properties of the eye of the subject and/or patient. The processor can be further configured to adjust one or more operating conditions of the Lorentz force motor described above, based on the determined characteristics, parameters, and/or properties of the eye of the subject and/or patient. As described above, the one or more characteristics and/or properties of the eye of the subject and/or patient can include a thickness of a sclera layer, a thickness of a choroid layer, a thickness of a retina (e.g., a retinachoroidal tissue), a permeability of the sclera layer, a permeability of the choroid layer, a permeability of a retina, a distance and/or depth between the outer surface of the eye of the subject and/or patient and an interface between the sclera layer and the choroid layer, a density of the sclera, the choroid, the retina and/or other tissue layers, an electrical and/or an ionic impedance of the sclera, the choroid, the retina and/or other tissue layers, an optical impedance, reflectivity, and/or absorbance of the sclera, the choroid, the retina and/or other tissue layers, an acoustic impedance, reflectivity and/or an absorbance of the sclera, the choroid, the retina and/or other tissue layers, a thermal impedance, reflectivity, and/or absorbance of the sclera, the choroid, the retina and/or other tissue layers, and/or a distance and/or depth between the outer surface of the eye of the subject and/or patient and an interface between the choroid layer and the retina. In some implementations the control unit 150 can adjust the operating conditions of the Lorentz motor based on the determined characteristics, parameters, and/or properties of the eye of the subject and/or patient as well on as the type of nozzle of the injector 130 (e.g., nozzle size, shape, and other technical specifications). For example, as described above, the control unit 150 can adjust the type of current to be passed through the coil of the Lorentz force motor (e.g., an AC current or a DC current), the magnitude of the current passed on the coil, the frequency of an AC current passed through the coils, and/or the length that along an axis that the magnet is allowed to move in response to the magnetic force generated by the Lorentz force motor. The control unit 150 can interact with the injector 130 to adjust the operating conditions for the Lorentz force motor and then produce the flow of the medicinal component(s). The flow of the medicinal component(s) can be directed to the nozzle of the delivery device 100 to generate a stream having a predetermined width of the stream, velocity of the medicinal component(s) in the stream, and/or trajectory of the stream towards the eye of the subject and/or patient, as described above.

In some implementations, the control unit 150 can be operably coupled to the injector 130 to adjust and/or control the operation of the magnetic levitation device of the injector 130. In such implementations the control unit 150 can be configured to control and/or define one or more operating conditions of the magnetic levitation device, including, but not limited to, a magnitude of a current passed through the magnetized coils, a frequency with which current is passed through the magnetized coils, and/or the strength of the alternating magnetic field.

In use, the control unit 150 can receive one or more signals from the sensor(s) 140, with the signals being associated with one or more characteristics, parameters, and/or properties of the choroid, and/or retina tissue layers of the eye of the subject and/or patient (either each layer by itself, or in combination), as measured by the sensor(s) 140. The control unit 150 can include a processor and a transducer configured to receive and analyze the signals received from the sensor(s) 140 (e.g., electrical, optical, acoustic, thermal, and/or mechanical signals) and determine, based on the signals received, the one or more characteristics, parameters, and/or properties of the eye of the subject and/or patient. The processor can be further configured to adjust one or more operating conditions of the magnetic levitation device described above, based on the determined characteristics, parameters, and/or properties of the eye of the subject and/or patient. As described above, the one or more characteristics and/or properties of the eye of the subject and/or patient can include a thickness of a sclera layer, a thickness of a choroid layer, a thickness of a retina (e.g., a retinachoroidal tissue), a permeability of the sclera layer, a permeability of the choroid layer, a permeability of a retina, a distance and/or depth between the outer surface of the eye of the subject and/or patient (e.g., including or excluding the conjunctiva) and an interface between the sclera layer and the choroid layer, a density of the sclera, the choroid, the retina and/or other tissue layers, an electrical and/or an ionic impedance of the sclera, the choroid, the retina and/or other tissue layers, an optical impedance, reflectivity, and/or absorbance of the sclera, the choroid, the retina and/or other tissue layers, an acoustic impedance, reflectivity and/or an absorbance of the sclera, the choroid, the retina and/or other tissue layers, a thermal impedance, reflectivity, and/or absorbance of the sclera, the choroid, the retina and/or other tissue layers, and/or a distance and/or depth between the outer surface of the eye of the subject and/or patient and an interface between the choroid layer and the retina. In some implementations the control unit 150 can adjust the operating conditions for the magnetic levitation device based on the determined characteristics, parameters, and/or properties of the eye of the subject and/or patient as well as on the type of nozzle of the injector 130 (e.g., nozzle size, shape, and other technical specifications). For example, as described above, the control unit 150 can adjust the magnitude of a current passed through the magnetized coils, and/or the frequency with which current passed through the magnetized coils. The control unit 150 can interact with the injector 130 to adjust the operating conditions of the magnetized levitation device and then produce the flow of the medicinal component(s). The flow of the medicinal component(s) can be directed to the nozzle of the injector 130 to generate a stream having a predetermined width of the stream, velocity of the medicinal component(s) in the stream, and/or trajectory of the stream towards the eye of the subject and/or patient, as described above.

The sensor(s) 140 can include any suitable device configured to sense and/or detect one or more properties, characteristics, and/or parameters of an eye of subject and/or patient. The sensor(s) 140 can be disposed and/or accommodated inside the housing 110. In some implementations, the sensor(s) 140 can be partially disposed inside the housing 110. Said in other words, in some implementations a first portion of the sensor(s) 140 can be disposed inside the housing 110, and a second portion of the sensor(s) 140 can be coupled to the housing 110 and/or disposed on a surface of the housing 110. In some implementations, the sensor(s) 140 can be disposed outside and/or separate from the housing 110, and operatively (e.g., communicatively) coupled to the device 110 (or more specifically, the control unit 150). The sensor(s) 140 can be electrically and/or operatively coupled to the control unit 150 such that the control unit 150 can activate, operate, and/or control the sensor(s) 140. In some implementations the sensor(s) 140 can receive from the control unit 150 a set of instructions and/or commands to measure and/or estimate a property of an eye of a subject and/or patient. For example, the sensor(s) 140 can receive a set of instructions and/or commands to produce, generate, and/or direct one or more excitation signals and/or stimuli to a region and/or portion of the eye of a subject and/or patient. In response to receiving the set of instructions, the sensor(s) 140 can produce the excitation signals and/or stimuli. The sensor(s) 140 can direct the excitation signals and/or stimuli to a portion and/or region of an eye of a subject and/or patient. The sensor(s) 140 can then detect and/or sense one or more response signals from the eye of the subject and/or patient, with the response signals being produced in response to the excitation and/or stimuli signals. The sensor(s) 140 can transmit the detected and/or sensed response signals (or data associated to the response signals) to the control unit 150. The control unit 150 can receive the response signals (or the data associated to the response signals) and determine via a processor of the control unit 150, one or more characteristics and/or properties of the eye of the subject and/or patient based on the detected and/or sensed signals (or the data associated to the detected and/or sensed signal). For example, in some implementations, the one or more characteristics and/or properties of the eye of the subject and/or patient can include a thickness of a sclera layer, a thickness of a choroid layer, a thickness of a retina (e.g., a retinachoroidal tissue), a permeability of the sclera layer, a permeability of the choroid layer, a permeability of a retina, a distance and/or depth between the outer surface of the eye of the subject and/or patient and an interface between the sclera layer and the choroid layer, a density of the sclera, the choroid, the retina and/or other tissue layers, an electrical and/or an ionic impedance of the sclera, the choroid, the retina and/or other tissue layers, an optical impedance, reflectivity, and/or absorbance of the sclera, the choroid, the retina and/or other tissue layers, an acoustic impedance, reflectivity and/or an absorbance of the sclera, the choroid, the retina and/or other tissue layers, a thermal impedance, reflectivity, and/or absorbance of the sclera, the choroid, the retina and/or other tissue layers, and/or a distance and/or depth between the outer surface of the eye of the subject and/or patient and an interface between the choroid layer and the retina.

In some implementations the sensor(s) 140 can be and/or include one or more optical sensor(s) (not shown). The optical sensor(s) can emit excitation signals directed towards an eye of a subject and/or patient and detect and/or sense response signals produced in response to the excitation signal. The optical sensor(s) can include, but are not limited to, a photodiode, a light emitting diode (LED), an LED coordinated with a photosensor (e.g., photodetector), a fiber optic sensor (e.g., fiber optic strand, fiber Bragg Grating sensor, fluoroptic sensor, and the like), and/or a flexible photonic sensor. In some implementations the fiber optic sensor can include and intrinsic sensor, where the fiber optic sensor itself represents the sensing element; or an extrinsic fiber optic sensor, where the fiber optic sensor acts as a conveyor of light, which is measured by a separate detector. The optical sensor can be configured to emit and/or detect one or more light signals (e.g., light signals having different wavelengths). For example, in some implementations the optical sensor(s) can include a near infrared light sensor, an infrared light sensor, a visible light sensor, and/or an ultraviolet light (e.g., fluoresce) sensor. The optical sensor(s) can be configured to detect and/or sense at least one of transmitted light and/or reflected light signals.

In use, the optical sensor can receive a set of instructions and/or commands from the control unit 150 to emit an excitation light signal. The optical sensor can direct the excitation light signal to a portion and/or region of the eye of the subject and/or patient. The excitation light signal can be reflected, absorbed, and/or transmitted by the portion and/or region of the eye of the subject and/or patient. The reflected, absorbed, and/or transmitted light can produce and/or generate a response light signal that can be detected by the optical sensor. The light absorbance and/or transmittance determined by comparing the excitation light signal and the response light signal can be used to determine one or more properties of the portion and/or region of the eye of the subject and/or patient. For example, in some implementations the light absorbance and/or transmittance can be used to determine a thickness, density, and/or permeability of one or more layers and/or tissues of the eye of the subject and/or patient.

In some implementations the sensor(s) 140 can be and/or include an ocular tonometry sensor (not shown). The tonometry sensor can be used to exert an excitation force on a surface of an eye of a subject and/or patient. The tonometry sensor can detect and/or sense an opposing force exerted by the eye of the subject and/or patient in response to the excitation force. The detected and/or sensed opposing force can be used to determine one or more characteristic of the eye of the subject and/or patient including, for example, intraocular pressure (IOP). In some implementations, the ocular tonometry sensor can be configured to measure the IOP via applanation tonometry. In such implementations, the IOP is inferred from the force required to flatten (applanate) a constant area of the cornea. In some implementations, the ocular tonometry sensor can be configured to measure the IOP via Goldman tonometry. In such embodiments, the tonometer includes a prism mounted on the head of the tonometer and placed against the cornea of the eye of a subject and/or patient, an optical filter, and a dial connected to a variable tension spring used to adjust the force exerted by the tonometer.

In use, the force applied to the tonometer head is adjusted using a dial until a mark such as one or more semicircles produced by the filter matches and/or overlaps in the viewfinder of the tonometer, which mark the flattening of a predetermined area of the cornea. At that position, the opposing forces of corneal rigidity and the tear film are roughly approximate and cancel each other out allowing the pressure in the eye to be determined from the force applied. In some implementations the tonometer can be configured to conduct one or more methods to determine intraocular pressure (IOP) and/or other characteristics and/or properties of the eye of the subject and or patent. For example, in some implementations the tonometer can be configured to conduct dynamic contour tonometry (DCT), electronic indentation tonometry, rebound tonometry, pneumatonometry, impression tonometry, non-corneal and transpalpebral tonometry, non-contact tonometry, and/or ocular response analysis (ORA). The tonometer can be used to estimate and/or measure the mechanical resistance of the sclera and other ocular tissue layers to an applied force, and determine, based on the measurements, one or more characteristics of the eye of the subject such as a thickness, a density, and/or a permeability of one or more layers and/or tissues of the eye of the subject and/or patient. The tonometer can also be used to determine changes to those characteristics intra-procedure, e.g., to determine when a tissue, such as the sclera, has been sufficiently manipulated to receive a medicament from the medicament container 120.

In some implementations the sensor(s) 140 can be and/or include an interferometer (not shown) including, but not limited to, a low-coherence interferometer. The interferometer can be configured to conduct one or more imaging methods such as optical coherence tomography (OTC) (e.g., OTC interferometer). The OTC interferometer can generate an excitation optical beam and direct the excitation optical beam to a receiving surface of an ocular tissue of an eye of a subject and/or patient. A first portion of the light included in the excitation optical beam can be scatter at large angles by the ocular tissue of the eye, while a second (much smaller) portion of the light can be reflected by a layer of the ocular tissue located underneath the receiving surface. The OTC interferometer can detect and/or sense a response light beam reflected in response to the excitation optical beam. The OTC interferometer can also be configured to determine the optical pathlength of photons received to generate a three-dimensional image of the ocular tissue later of the eye.

In use, the OTC interferometer can receive electrical signals associated with a set of instructions and/or commands from the control unit 150 to generate one or more excitation optical beams. The OTC interferometer can be configured to direct the excitation optical beam to a portion and/or region of the eye of the subject and/or patient. The excitation optical beam can interact with the portion and/or region of the eye of the subject and/or patient and generate a response beam (e.g., a reflected light beam). The OCT interferometer can also detect, sense and/or measure the response beam and determine the optical pathlength of photons received (allowing to reject photons that scatter multiple times before detection) to produce high quality 3D images of the ocular tissue layers of the eye of the subject and/or patient. As described above, the measured OTC interference can be used to determined and/or estimate a small distance, a refractive index, and/or a surface irregularity, which in turn can be used to determine a thickness, density, and/or permeability of one or more layers and/or tissues of the eye of the subject and/or patient.

In some implementations the sensor(s) 140 can be and/or include one or more electrodes. The one or more electrodes can be configured to produce an excitation electrical signal (e.g., a voltage and/or a current) on a surface of an eye of a subject and/or patient. The one or more electrodes can also detect and/or sense a response electrical signal (e.g., a voltage and/or a current) produced on the surface of an eye of a subject and/or patient in response to the excitation electrical signal.

In some implementations the electrodes can include one or more excitation electrodes and one or more sensing electrodes. In some implementations the electrodes can include a set of electrodes, with each electrode of the set of electrodes being configured to serve as either an excitation electrode or a sensing electrode. In some implementations the set of electrodes can include excitation electrodes, designated to deliver excitation signals to a portion and/or region of the eye of the subject and/or patient, and sensing electrodes designated to sense a response signal from the portion and/or region of the eye in response to an application of an excitation signal. In some implementations, the electrodes can be configured to generate an excitation current signal in the form of direct current (DC). In some implementations, the electrodes are configured such that the generated excitation current signal is in the form of alternating current (AC). In some implementations the electrodes can have a carbon coating to reduce contamination and/or corrosive effects of the surface of the eye (due to salinity, and/or other bodily fluids present on the surface of the eye of the subject and/or patient).

In use, the electrode (or a set of electrodes) can receive a set of instructions and/or commands from the control unit 150 to generate an excitation electrical signal (e.g., a voltage and/or a current) on a surface of the eye of the subject and/or patient. The excitation electrical signal can interact with the eye of the subject and/or patient and generate a response electrical signal (e.g., a voltage and/or current) that can be detected by the electrodes (or the set of electrodes). The response electrical can be used to measure one or more characteristics of the eye of the subject and/or patient, including an electrical resistance, an electrical impedance, and/or an ionic impedance. The resistance and/or impedances measured by the electrodes can be used to determine a thickness, density, and/or permeability of one or more layers and/or tissues of the eye of the subject and/or patient.

In some implementations the sensor(s) 140 can be and/or include an ultrasonic sensor. The ultrasonic sensor can be configured to emit an excitation sound wave directed towards an eye of a subject and/or patient and detect and/or sense response sound wave produced in response to the excitation sound waves. The ultrasonic sensor can include a transmitter and a receiver (not shown). The transmitter can include a piezoelectric crystal configured to emit excitation sound waves directed to the eye of a subject and/or patient. The receiver can encounter and/or detect response sound waves produced in response to the excitation sound waves after the excitation sound waves have travelled from the transmitter to a target region or portion of the eye, such as the sclera tissue, and then back to the ultrasonic sensor. The ultrasonic sensor can measure a distance between the ultrasonic sensor an object such as the interface between the sclera and choroid (e.g., the suprachoroidal space SCS) by measuring the time that passes between the emission of the excitation sound wave by the transmitter and the detection of the response sound wave by the receiver. In use, the ultrasonic sensor can receive a set of instructions and/or commands from the control unit 150 to emit an excitation sound wave. The ultrasonic sensor can direct the excitation sound wave to a portion and/or region of the eye of the subject and/or patient. The excitation sound wave can be reflected by the portion and/or region of the eye of the subject and/or patient. The reflected sound wave can be detected by the receiver. The ultrasonic sensor can then determine one or more properties of the portion and/or region of the eye of the subject and/or patient. For example, in some implementations the response from the ultrasonic sensor can be used to determine a thickness, density, and/or permeability of one or more layers and/or tissues of the eye of the subject and/or patient.

The control unit 150 can be configured to activate and/or control the operation of one or more components of the delivery device 100, e.g., by receiving electrical signal(s) from and/or sending electrical signal(s) to other components of the delivery device 100. The control unit 150 can include a memory, a processor, and an input/output (I/O) device (not shown).

The memory can be, for example, a random-access memory (RAM), a memory buffer, a hard drive, a database, an erasable programmable read-only memory (EPROM), an electrically erasable read-only memory (EEPROM), a read-only memory (ROM), and/or so forth. In some implementations, memory stores instructions that cause processor to execute modules, processes, and/or functions associated with operating one or more components of the delivery device 100. Such instructions can be designed to integrate specialized functions into the control unit, such that the delivery device 100 can perform methods, as further described below

The processor of the control unit 150 can be any suitable processing device configured to run and/or execute functions associated the delivery device 100. For example, processor can be configured to process and/or analyze sensor data (e.g., received from sensor(s) 140), to cause the injector 130 to generate a stream of medicinal component(s) stored in the medicament container 120, to adjust one or more parameters of the injector 130 (e.g., a pressure, an electrical current, a voltage, a frequency, amplitude, etc.), and to generate feedback and/or instructions to provide to a healthcare professional and/or a user to facilitate application of the medicinal component(s) in to the eye of a subject and/or patient using the delivery device 100, as further described herein. The processor 154 can be a general-purpose processor, a Field Programmable Gate Array (FPGA), an Application Specific Integrated Circuit (ASIC), a Digital Signal Processor (DSP), and/or the like

The input/output (I/O) device includes one or more components for receiving information and/or sending information to other components of delivery device 100 and/or other devices. In some embodiments, the I/O device can optionally include or be operatively coupled to a display, audio device, or other output device for presenting information to a healthcare professional and/or a user. In some implementations, the I/O device can include a communication interface (not shown) that can enable communication between control unit 150 and the medicament container 120, the one or more of sensor(s) 140, the injector 130, and/or the optional tissue manipulation component 160. In some implementations, the I/O device can include a network interface that can enable communication between control unit 150 and one or more external devices, including, for example, an external user device (e.g., a mobile phone, a tablet, a laptop) and/or other compute device (e.g., a local or remote compute, a server, etc.). The network interface can be configured to provide a wired connection with the external device, e.g., via a port or firewall interface, can be configured to communicate with the external device via a wireless network (e.g., Wi-Fi, Bluetooth®, low powered Bluetooth®, Zigbee and the like). In some embodiments, the communication interface can also be used to recharge a power source (e.g., a rechargeable battery).

In some embodiments, the control unit 150 can be or form part of an integrated circuit. For example, the control unit 150 can be an integrated chip that integrates components including a memory, a central processing unit, and one or more ports and/or interfaces for communication (e.g., I/O device).

The control unit 150 can generate electrical signals to control one or more components of the delivery device 100, e.g., based on instructions stored in the memory. These electrical signals may be communicated between the control unit 150 and other components of the delivery device 100 via a communication interface of I/O device. In some implementations, the control unit 150 can control the injector 130 to generate a stream of the medicinal component(s) stored and/or contained in the medicament compartment 120, and direct and/or guide the generated stream of medicinal component(s) (via the nozzle of the injector 130) towards one or more predetermined regions and/or portions of an eye of a subject and/or patient. For example, in some implementations the control unit 150 can control a high-pressure chamber of the injector 130 which can be transitioned from a first configuration and/or state to a second configuration and/or state to move a movable wall and/or piston and generate a flow of medicinal component(s) directed to the nozzle of the injector 130. In some implementations the control unit 150 can control a Lorentz motor of the injector 130 which can be engaged to generate an oscillating magnetic field that can be used to move a movable component of the injector 130 such as a wall and/or a piston and generate a flow of medicinal component(s) directed to the nozzle of the injector 130. In some implementations the control unit 150 can control a Lorentz driver and/or actuator of the injector 130 which can be engaged to generate a magnetic field that can directly interact with the medicinal component(s) stored inside the medicament container 120 and generate a flow of medicinal component(s) directed to the nozzle of the injector 130. In some implementations, the control unit 150 can adjust one or more parameters of the injector 130 (e.g., a pressure, an electrical current, a voltage, a frequency, amplitude, etc.) based on signals or data received from the sensor(s) 140. For example, the sensor(s) 140 can be configured to measure a light signal, an exerted force, a voltage, a current, an impedance, or the like, and the control unit 150 can control the operation of the injector 130 and/or the tissue manipulation component 160 (e.g., by sending electrical signals to the injector 130 and/or the tissue manipulation component 160) based on data received from the sensors 140.

The optional tissue manipulation component 160 can be any suitable device configured to interact with one or more ocular tissue layers of an eye of a subject and/or patient to increase the ocular tissue layer permeability to the medicinal component(s), facilitating delivery of the medicinal component(s) to a target region and/or portion of the eye of the subject and/or patient. FIG. 5 shows the tissue manipulation component 160 can be coupled to the control unit 150. Although the tissue manipulation component 160 is shown disposed within the housing 110, in some embodiments the tissue manipulation component 160 may be disposed outside and/or separate from the housing 110. Furthermore, in some implementations the tissue manipulation component 160 can include or be included within a kit, e.g., including multiple components such as one or more reservoirs containing chemical reagents, active ingredients, and/or other species to initiate chemical and/or physical transformations on ocular tissue layers, and one or more devices for delivering such chemical reagents (e.g., microinjectors, needles, and/or topical delivery devices). In some implementations the tissue manipulation component 160 can be operatively coupled to the control unit 150 to activate, and/or control the operation of the tissue manipulation component 160. For example, in some implementations the tissue manipulation component 160 can receive one or more electrical signals associated with a set of instructions to adjust one or more parameter of the tissue manipulation component 160 such as a wavelength, a frequency, a pulse length, a pulse energy, a number of pulses, an exposure time (e.g., the duration of an interaction), and/or a pulse repetition.

In some implementations, the tissue manipulation component 160 can include one or more chemical species that can initiate chemical reactions and/or physical transformation(s) on a specific ocular tissue layer such as the sclera, to increase the permeability and/or diffusivity of medicinal component(s) in the sclera. In some implementations the tissue manipulation component 160 can include chemical species that can initiate chemical reactions and/or physical transformation(s) on multiple ocular tissue layers (e.g., the sclera, cornea, choroid, limbus, conjunctiva, and/or the retina), to increase the permeability and/or diffusivity of medicinal component(s) in the ocular tissue layers. In such implementations, the tissue manipulation component 160 can include a reservoir (not shown) sized and shaped to contain the chemical species, and a chemical reagent injector (not shown), as further described herein. In some implementations the reservoir of the tissue manipulation component 160 can be integrated and/or incorporated in the medicament container 120. For example, in some implementations the reservoir can be one or more chambers and/or compartments of the medicament container 120, which are sized and shaped to accommodate the chemical species required to initiate chemical reactions and/or physical transformations on the tissue layers of the eye of the subject and/or patient. In other implementations, the reservoir of the tissue manipulation component 160 can be a stand-alone reservoir disposed within the housing 110 and configured to store and/or accommodate the chemical species of the tissue manipulation component 160. In some implementations, the reservoir can be disposed separate from the housing 110.

The chemical reagent injector of the tissue manipulation component 160 can be any suitable component configured to (1) generate a stream of the chemical species required to initiate chemical reactions and/or physical transformations on the ocular tissue layers of the eye of the subject and/or patient, and (2) direct the generated stream of the chemical species to the ocular tissue layer(s) of the eye of the subject and/or patient. In some implementations the chemical reagent injector of the tissue manipulation component 160 can be integrated and/or incorporated in the injector 130. In such implementations the injector 130 serves a dual purpose. In some instances, the injector 130 can generate a stream of medicinal component(s) and direct and/or guide (via the nozzle) the generated stream of medicinal component(s) to a target region of the eye of the subject and/or patient. In other instances, the injector 130 can generate a stream of the chemical species required to initiate the chemical reactions and/or physical transformations, and direct and/or guide the chemical species to the ocular tissue layer(s) of the eye of the subject and/or patient. As described above, the tissue manipulation component 160 can be coupled to the control unit 150 such that the control unit 150 can control the operation of the tissue manipulation component 160. In some implementations, the control unit 150 can be configured to send electrical signals to the tissue manipulation component 160 to control an amount of chemical species (e.g., a volume, a mass, etc.) directed to the eye of the subject and/or patient, and/or a frequency of delivering the chemical species to the eye of the subject and/or patient.

The chemical species included in the tissue manipulation component 160 can be any suitable reagent that can interact (chemically and/or physically) with one or more components of the different ocular layers of an eye to increase the permeability and/or diffusivity of the medicinal component(s) within an eye of a subject and/or patient. For example, in some implantations the chemical species of tissue manipulation component 160 can include agent effective to degrade collagen or glycosaminoglycan (GAG) fibers in the sclera or corneal stroma, which may enhance penetration/release of the medicinal component(s) into the ocular tissues. This agent may be, for example, an enzyme, such a hyaluronidase, a collagenase, or a combination thereof (e.g., a collagen degradation agent). In some implementations the chemical species can include a topical anesthetic species formulated to prevent and/or minimize pain during the procedure, as well as to aid increasing the permeability of the sclera layer. In some implementations, the anesthetic species can be compounded with the collagen degradation agent. In use, the tissue manipulation component 160 of the delivery device 100 can be used to deposit the anesthetic species and the collagen degradation agent under the conjunctiva tissue layer to initiate the chemical reactions to increase the permeability of the sclera tissue.

In some implementations the tissue manipulation component 160 can be any suitable device configured to impart mechanical and/or thermal perturbations to one or more ocular tissue layers (e.g., the cornea, sclera, choroid, limbus, conjunctiva, and/or the retina) to increase the permeability and/or diffusivity of medicinal component(s). For example, in some implementations the tissue manipulation component 160 can include an ultrasound actuator (not shown). The ultrasound actuator can be a device configured to contact an external surface of an eye of a subject and/or patient and apply an ultrasound perturbation at a frequency having sufficient intensity to reduce the resistance of the ocular tissue layers (e.g., the sclera).

In some implementations the ultrasound actuator can be configured to apply a frequency of no more than about 16 MHZ, no more than about 14 MHZ, no more than about 12 MHz, no more than about 10 MHz, no more than about 8 MHZ, no more than about 6 MHz, no more than about 4 MHZ, no more than about 2 MHz, no more than about 1 MHZ, no more than about 500 kHz, no more than about 300 kHz, no more than about 100 kHz, no more than about 50 kHz, no more than about 20 kHz, inclusive of all values and ranges therebetween. In some implementations the ultrasound actuator can be configured to apply a frequency of at least about 20 kHz, at least about 50 kHz, at least about 100 kHz, at least about 200 kHz, at least about 400 kHz, at least about 600 kHz, at least about 800 kHz, at least about 1 MHZ, at least about 2 MHZ, at least about 4 MHZ, at least about 8 MHz, at least about 12 MHZ, at least about 16 MHz. Combinations of the above-referenced ranges for the frequencies of the ultrasound actuator are also possible (e.g., at least about 20 kHz and no more than 16 MHZ, or at least about 500 kHz and no more than 8 MHz).

In some implementations the tissue manipulation component 160 can include a thermal ablation device (not shown). The thermal ablation device can be configured to heat a small area of an ocular tissue layer of an eye of a subject and/or patient to deplete the ocular tissue layer at a that site without significantly damaging ocular tissue disposed underneath the exposed area. In some implementations the thermal ablation device can include a laser gun, a radiofrequency (RF) device, and/or an electrical heating element (not shown). For example, in some implementations the thermal ablation device can include a laser gun configured to deposit optical energy over a small and/or controlled area of an ocular tissue (e.g., the sclera), causing evaporation of water and formation of microchannels within the sclera that can facilitate the diffusion of a stream of medicinal component(s). In some implementations the laser gun of the tissue manipulation component 160 can be operatively coupled to the control unit 150 to activate, and/or control the operation of the laser gun. In such implementations, the laser gun can receive one or more electrical signals from the control unit 150 associated with a set of instructions to adjust one or more parameter of the laser gun such as a wavelength, a frequency, a pulse length, a pulse energy, a number of pulses, an exposure time (e.g., the duration of an interaction), and/or a pulse repetition. In some implementations, the tissue manipulation component 160 and/or the laser gun can include an end effector configured to contact the conjunctiva tissue layer and prevent undesired absorption of the optical energy by water in the conjunctiva tissue layer (e.g., the conjunctiva tissue layer can include about 80% water). The end effector can disrupt the conjunctiva tissue layer and expose a surface of the sclera tissue layer located directly underneath. In that way, the laser gun can direct the optical energy directly to the sclera and avoid absorption of energy by water molecules in the conjunctiva tissue layer.

In some implementations, an opening can be formed in the conjunctiva to provide direct access to the sclera. The conjunctiva is typically under tension, and so an operator can leverage that tension to create a suitable opening in the conjunctiva by performing a small incision in the conjunctiva.

As described above, in some implementations the thermal ablation device can include a radiofrequency (RF) device. In such implementations, the RF device can include a thin probe and/or needle electrode that can be placed directly into an ocular layer (e.g., the sclera, and/or other tissue layers such as the cornea, sclera, choroid, limbus, conjunctiva, and/or others) of an eye of a subject and/or user. The RF device can apply and/or pass a high frequency alternating (AC) current thought the ocular tissue layer(s) to produce and/or open microscopic pathways within the ocular tissue layer that can be used to diffuse medicinal component(s) In some implementations the RF device can be configured to apply an AC current at frequency of no more than about 500 kHz, no more than about 450 kHz, no more than about 400 kHz, no more than about 350 kHz, no more than about 300 kHz, no more than about 250 kHz, no more than about 200 kHz no more than about 180 kHz, no more than about 160 kHz no more than about 140 kHz, no more than about 120 kHz, no more than about 100 kHz, inclusive of all values and ranges therebetween. In some implementations the RF device can be configured to apply an AC current at frequency of at least about 100 kHz, at least about 140 kHz, at least about 180 kHz, at least about 220 kHz, at least about 260 kHz, at least about 300 kHz, at least about 340 kHz, at least about 380 kHz, at least about 420 kHz, at least about 460 kHz, at least about 500 kHz. Combinations of the above-referenced ranges for the AC current at frequencies of the RF device are also possible (e.g., at least about 10 kHz and no more than 500 kHz, or at least about 280 kHz and no more than 450 kHz).

In some implementations the tissue manipulation component 160 can include an iontophoresis device (not shown). The iontophoresis device can include one or more electrodes (e.g., a cathode and an anode electrode) that can be disposed on an external surface of an eye of a subject and/or patient. The electrodes can be coupled to a power source (not shown) that can establish a potential (e.g., a voltage) between the electrodes and apply and/or pass an excitation DC current (at a relatively low current density in mA/cm²) on the eye of the subject and/or patient. The excitation DC current can interact with charged species disposed on the ocular tissue layers of the eye such that positively charged species (e.g., cationic species) disposed under the anode electrode are driven away into the inner ocular tissue layers as they are repelled by the anode electrode. Similarly, negatively charged species (e.g., anionic species) disposed on inner ocular tissue layers are attracted to the cathode electrode. In use, medicinal component(s) having an effective charge (e.g., cationic and/or anionic species) can be disposed on an external surface of an eye of a subject and/or patient. For example, cationic and/or anionic medicinal component(s) can be disposed on an external surface of an eye of a subject and/or patient using the injector 130 of the delivery device 100, as described above. The anode electrode of the iontophoresis device can then be disposed on an external surface of the eye in which cationic medicinal components have been deposited. The cathode electrode of the iontophoresis device can be disposed on an external surface of the eye in which anionic medicinal components have been deposited. The iontophoresis device can then be controlled by the control unit 150 to pass a low current on the eye of the subject and/or patient and cause diffusion of the medicinal components due to their electrical attraction/repulsion to the electrodes. In some implementations, the control unit 150 can be configured to send electrical signals to the tissue manipulation component 160 to control the magnitude of the DC excitation current passed, the duration of the excitation DC current, and/or the frequency with which the DC excitation current is applied.

In some implementations the iontophoresis device can be configured to apply an DC current of no more than about 1 mA/cm², no more than about 0.9 mA/cm², no more than about 0.8 mA/cm², no more than about 0.7 mA/cm², no more than about 0.6 mA/cm², no more than about 0.5 mA/cm², no more than about 0.4 mA/cm², no more than about 0.3 mA/cm², no more than about 0.2 mA/cm², no more than about 0.1 mA/cm², inclusive of all values and ranges therebetween. In some implementations the iontophoresis device can be configured to apply an DC current of at least about 0.1 mA/cm², at least about 0.15 mA/cm², at least about 0.2 mA/cm², at least about 0.25 mA/cm², at least about 0.3 mA/cm², at least about 0.35 mA/cm², at least about 0.4 mA/cm², at least about 0.45 mA/cm², at least about 0.5 mA/cm², at least about 0.55 mA/cm², at least about 0.6 mA/cm², at least about 0.65 mA/cm², at least about 0.7 mA/cm², at least about 0.75 mA/cm², at least about 0.8 mA/cm², at least about 0.85 mA/cm², at least about 0.9 mA/cm², at least about 0.95 mA/cm², at least about 1 mA/cm². Combinations of the above-referenced ranges for the DC current of the iontophoresis device are also possible (e.g., at least about 0.1 mA/cm² and no more than 1 mA/cm², or at least about 0.25 mA/cm² and no more than 0.95 mA/cm²)

In some implementations the tissue manipulation component 160 can be, include, or be integrated or associated with a separate device such as an injector or microinjector configured to assist in the insertion of a delivery member (e.g., a needle, microneedle, a solid microneedle, a trocar, a catheter, a hollow trephine, cannula, microneedle patch, hollow microneedle, or hollow conduit) into the eye, or more specifically, a retinal space of the eye or a retinal tissue. The delivery member can be used to introduce chemical reagents that alter the salinity and/or electrical properties of the retinal tissue. For example, in some implementations the delivery member can introduce a chemical species having a net positive charge (cationic species) or a net negative charge (anionic species), ionic compounds such as organic salts, inorganic salts, buffer solutions, electrolytes, and the like. The chemical species can be dispersed on the retinal tissue to change the electrical properties of the retina, and/or to generate a layer of electrically charged species (thus generating an electrical potential between the outer surface of the eye and the retinal tissue). In use, the delivery member can be used to deposit one or more chemical species to the retinal tissue. The delivery device 100 can then be used to direct medicinal component(s) to an ocular tissue layer disposed over the retina (e.g., an outer surface of the eye of the patient and/or subject, or an outer tissue layer such as the cornea, the conjunctiva, and/or the sclera). The medicinal component(s) can be selected such that they exhibit a net electrical charge opposite to the net electrical charge of the chemical species introduced via the delivery member. For example, the medicinal component(s) be and/or include cationic and/or anionic species. The medicinal components can diffuse towards the retinal tissue layer driven by the attraction between opposite charges present on the medicinal component and the chemical species in the retinal tissue.

The optional contact member 170 can be any suitable component that can be disposed in direct contact with an external surface of an eye of a subject and/or patient. The contact member 170 can be configured to temporarily adhere (e.g., atraumatically) to the external surface of the eye and prevent and/or minimize movement of the delivery device 100 while the delivery device 100 is being operated to direct a stream of medicinal component(s) to portions and/or regions of the eye. The contact member 170 can be configured to contact an external tissue layer of the eye of the subject and/or patient (e.g., the conjunctiva or sclera of the eye) when the delivery device 100 is being used to convey medicinal component(s) to a target ocular tissue layer such as the sclera, the choroid and/or the retina. In some implementations, the contact member 170 can be configured to deform a surface (e.g., a surface of the conjunctiva of the eye) when the delivery 100 is placed in physical contact with the eye of the subject and/or patient. This surface can also be referred to herein as the “contact surface of the eye”. In some implementations, at least a portion of the contact member 170 can have a substantially convex shape, for example, a hemispherical shape such that at least a portion of the contact member 170 defines a sealing portion with the conjunctiva of the eye. The sealing portion can be configured to define a substantially fluid-tight seal with the contact surface of the eye when the contact member 170 is in contact with the contact surface of the eye. For example, the contact member 170 can deform the conjunctiva of the eye such that the sealing portion is contiguous with the contact surface of the eye and forms the substantially fluid-tight seal. In some embodiments, the sealing portion can be symmetric about an axis of symmetry of the medicament delivery device 100 (e.g., an axial axis of the delivery device 100). This can, for example, facilitate perpendicular approach of the injector 130 to facilitate generating a stream of medicinal component(s) aimed at a target ocular tissue.

The contact member 170 can have any suitable shape and/or form. In some implementations, the contact member 170 can be substantially cylindrical is shape. In such implementations, the contact member 170 can have a circular cross-section such that the convex shape of the contact member 170 resembles, for example, a hemisphere. The sealing portion generated by the contact member 170 can circumferentially surround the injector 130 to form a hemispherical substantially fluid-tight seal with the contact surface of the eye. Alternatively, in some embodiments the contact member 170 can have a conical, or a parabolic shape. In use, a medical professional and/or a user can apply pressure to the delivery device 100 against the contact surface of the eye of the patent and/or user to generate a fluid-tight seal with the contact surface of the eye.

The contact member 170 can be formed or constructed of one or more biocompatible materials. In some implementations, the biocompatible materials can be selected based on one or more properties of the constituent material such as, for example, stiffness, toughness, durometer, bioreactivity, etc. Examples of suitable biocompatible materials include metals, glasses, ceramics, or polymers. Examples of suitable metals include pharmaceutical grade stainless steel, gold, titanium, nickel, iron, platinum, tin, chromium, copper, and/or alloys thereof. A polymer material may be biodegradable or non-biodegradable. Examples of suitable biodegradable polymers include polylactides, polyglycolides, polylactide-co-glycolides (PLGA), polyanhydrides, polyorthoesters, polyetheresters, polycaprolactones, polyesteramides, poly (butyric acid), poly (valeric acid), polyurethanes, and/or blends and copolymers thereof. Examples of non-biodegradable polymers include nylons, polyesters, polycarbonates, polyacrylates, polysiloxanes (silicones), polymers of ethylene-vinyl acetates and other acyl substituted cellulose acetates, non-degradable polyurethanes, polystyrenes, polyvinyl chloride, polyvinyl fluoride, poly (vinyl imidazole), chlorosulphonate polyolefins, polyethylene oxide, and/or blends and copolymers thereof.

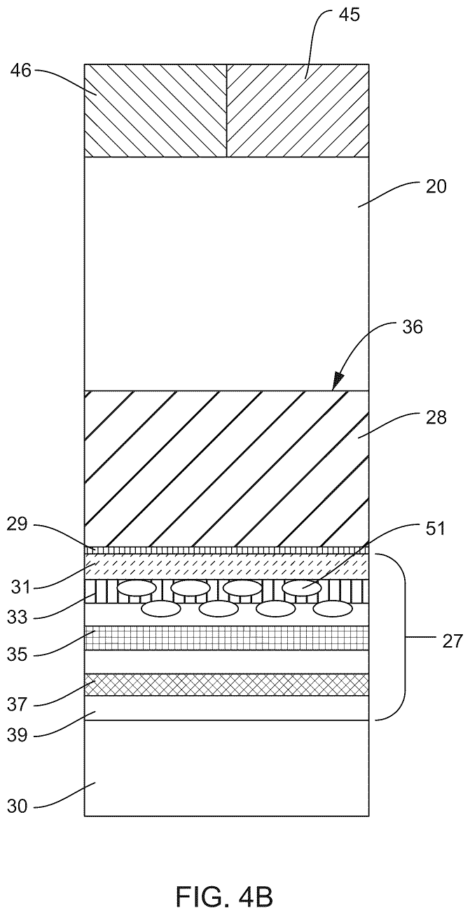

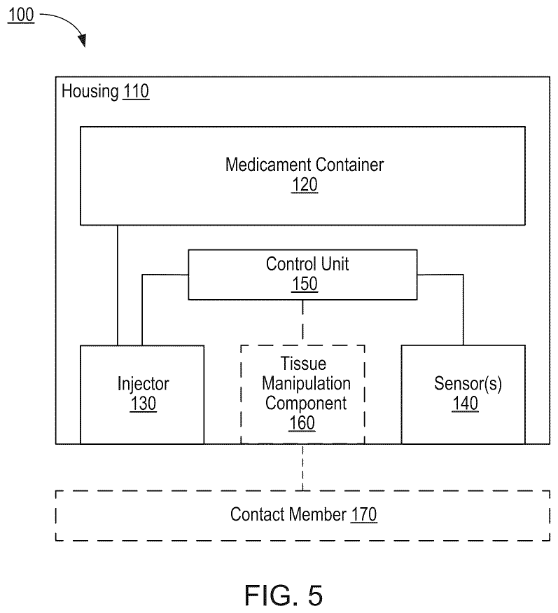

FIG. 6A illustrates a medicament delivery device 200 according to an embodiment.

The medicament delivery device 200 (also referred to herein as the “delivery device”) can be similar in form and/or function to the delivery device 100, described above. For example, as described above with reference to the delivery device 100, the delivery device 200 can be used to delivery one or more medicaments and/or medicinal components to specific portions and/or regions of a human eye without using a piercing member such as a needle, cannula, stylet, and/or catheter (e.g., needle-less medicament delivery). The delivery device 200 can include a housing 210, a medicament container (not shown), an injector 230, a sensor 240, a control unit (not shown), and a contact member 270. In some implementations, portions and/or aspects of the delivery device 200 can be similar to and/or substantially the same as portions and/or aspects of the delivery device 100 described above with reference to FIG. 5. Accordingly, such similar portions and/or aspects may not be described in further detail herein.

The housing 210 defines an internal volume, compartment, and/or chamber for accommodating and/or coupling the various components of the delivery device 200. The housing 210 can be an elongated member having a circular cross-sectional shape that defines an interior volume sufficient large to accommodate and/or support the components of the delivery device 200. Alternatively, in some implementations the housing 210 can be any suitable shape. In some implementations, the housing 210 can have a shape, surface features, and/or surface materials or finishes that can be configured to increase the ergonomics of the delivery device 200, which can, for example, allow a trained healthcare worker, a medical professional and/or a user to manipulate the delivery device 200 with one hand (i.e., single-handed use). The housing 210 can be made of any suitable material or materials having sufficient structural strength and rigidity, including, for example, metal, glass, ceramic, and/or polymers. In some implementations, the housing 210 can include multiple portions that can be coupled and/or assembled together to form the interior chamber and/or compartment for receiving the components of the delivery device 200. That is, in some implementations, the housing 210 can be modular. Alternatively, in other implementations, the housing 210 can be made of a monolithic structure.

FIG. 6A shows the injector 230 can be configured to (1) produce a flow of medicinal component(s) stored and/or contained in the medicament compartment of the delivery device 200, (2) direct and/or guide the produced flow of medicinal component(s) to the nozzle 232, and (3) generate, with the aid of the nozzle 232, a stream 234 of medicinal component(s) having one or more predetermined characteristics including: a width, a cross sectional area, a cross sectional shape, a velocity of the medicinal component(s), and/or a specific trajectory that transports the medicinal component(s) to a target region and/or portion of an eye of a subject and/or patient, such as the suprachoroidal space (SCS).

The injector 230 can include a high-pressure chamber (not shown) mechanically coupled to the medicament container of the delivery device 200. As described above with reference to the injector 130, the high-pressure gas chamber can contain one or more compressed gases (not shown) disposed inside the high-pressure chamber, and a movable component (not shown) such as a wall and/or a piston coupled to the medicament component of the delivery device 200. The compressed gases can be used to move the movable component of the injector 230 to exert a force on the medicament container (or a portion thereof). The forces exerted by the movable component of the injector 230 on the medicament container 120 can increase kinetic energy to the medicinal component(s) (e.g., accelerate the medicinal component(s) stored inside the medicament container of the delivery device 200). The high-pressure chamber can assume a first state and/or configuration (also referred to herein as a “compressed” configuration) in which the compressed gases are kept inside the high-pressure chamber occupying a first volume at a first pressure. The high-pressure chamber can be transitioned from the first state and/or configuration to a second state and/or configuration (also referred to herein as an “expanded” configuration). In the second state and/or configuration the compressed gases are allowed to expand and move the movable component (e.g., a movable wall and/or a piston) until the compressed gas is contained inside the high pressure chamber within a second volume and at a second pressure, the second volume being larger than the first volume, and the second pressure being lower than the first pressure The movement of the movable wall and/or piston can effectively apply a force on the medicament container of the delivery device 200 (or a portion thereof) such that the medicinal component(s) increase their kinetic energy and a flow of the medicinal component(s) is directed out of the medicament container of the delivery device 200 towards the nozzle 232. As discussed above, the nozzle 232 can receive the flow of medicinal component(s) and generate the stream 234 directed to the intended target region and/or portion of the eye of the subject and/or patient.

As described above with respect to the injector 130, in some implementations the injector 230 can be electrically and/or operatively coupled to the control unit included in the delivery device 200 to activate, and/or control the operation of the injector 230. The control unit can receive one or more signals from the sensor(s) 240, with the signals being associated with one or more characteristics and/or properties of the eye of the subject and/or patient measured by the sensor(s) 140. The control unit can include a processor (not shown) configured to analyze the signals received from the sensor(s) 240 and determine, based on the signals received, the one or more characteristics and/or properties of the eye of the subject and/or patient. The processor can be further configured to adjust the one or more parameters and/or operating conditions for the high-pressure chamber, based on the determined properties of the eye of the subject and/or patient. For example, the processor can be configured to determine, based on the signals received from the sensor(s) 240, a pressure (e.g., delivery pressure PDelivery) of the compressed gases contained in the high-pressure chamber of the injector 230 suitable to generate a stream of medicinal component(s) that can be directed to a target region and/or portion of an eye of a subject and/or patient.

FIG. 6B shows a chart and/or plot relating the delivery pressure (PDelivery) of the high-pressure chamber required to direct a stream of medicinal component(s) and penetrate one or more ocular tissue layers such as the sclera to deliver the medicinal component(s) at a target region of an eye of a subject, as a function of the measured thickness of the ocular tissues (e.g., the sclera tissue layer). In use, the processor can receive signals from the sensor(s) 240, with the signals being associated with one or more characteristics and/or properties of the eye of the subject and/or patient measured by the sensor(s) 240, as further described herein. The processor can use the signals to determine a thickness of the sclera layer of the eye of the subject and/or patient. The processor can use the chart or plot shown in FIG. 6 to determine, based on the measured sclera thickness, a suitable delivery pressure (PDelivery) to penetrate the sclera tissue and deliver the medicinal components to the suprachoroidal space (SCS) or any desired location beyond the sclera, such as, for example, the retina, vitreous, etc.

The sensor 240 can be disposed and/or accommodated inside the housing 210 with a portion of the sensor 240 being disposed at a distal portion of the housing 210, as shown in FIG. 6A. The sensor 240 can be electrically and/or operatively coupled to the control unit of the delivery device 200 such that the control unit can activate, operate, and/or control the sensor(s) 240. In some implementations the sensor(s) 240 can receive from the control unit a set of instructions and/or commands to measure and/or estimate a property of an eye of a subject and/or patient. The sensor 240 can be an IR optical sensor that can emit an IR excitation signals directed towards the eye of the subject and/or patient and detect and/or sense an IR response signals produced in response to the excitation signal. The IR signals can be used to determine via the processor one or more characteristics and/or properties of the eye of the subject such as a thickness of a sclera layer, a thickness of a choroid layer, a thickness of a retina (e.g., a retinachoroidal tissue), a permeability of the sclera layer, a permeability of the choroid layer, a permeability of a retina, a distance and/or depth between the outer surface of the eye of the subject and/or patient and an interface between the sclera layer and the choroid layer, and/or a distance and/or depth between the outer surface of the eye of the subject and/or patient and an interface between the choroid layer and the retina

The contact member 270 can be disposed and/or coupled to an outer surface of the housing 210, as shown in FIG. 6A. The contact member 270 can be placed in direct contact with an external surface of an eye of a subject and/or patient. The contact member 270 can adhere to the external surface of the eye and prevent and/or minimize movement of the delivery device 200 while the delivery device 200 is being operated to direct a stream of medicinal component(s) to portions and/or regions of the eye. The contact member 270 can be configured to contact an external tissue layer of the eye of the subject and/or patient (e.g., the conjunctiva or sclera of the eye) when the delivery device 200 is being used to convey medicinal component(s) to a target ocular tissue layer such as the sclera, the choroid and/or the retina. In some implementations, the contact member 270 can be configured to deform a surface (e.g., a surface of the conjunctiva of the eye) when the delivery 100 is placed in physical contact with the contact surface of the eye. In some implementations, at least a portion of the contact member 270 can have a substantially convex shape, for example, a hemispherical shape such that at least a portion of the contact member 270 defines a sealing portion with the conjunctiva of the eye. The sealing portion can be configured to define a substantially fluid-tight seal with the contact surface of the eye when the contact member 270 is in contact with the contact surface of the eye. For example, the contact member 270 can deform the conjunctiva of the eye such that the sealing portion is contiguous with the contact surface of the eye and forms the substantially fluid-tight seal. In some embodiments, the sealing portion can be symmetric about an axis of symmetry of the medicament delivery device 200 (e.g., an axial axis of the medicament delivery device 200). This can, for example, facilitate perpendicular approach of the injector 230 to facilitate generating a stream of medicinal component(s) aimed at a target ocular tissue. The contact member 270 can have any suitable shape and/or form. In some implementations, the contact member 270 can be substantially cylindrical is shape. In such implementations, the contact member 270 can have a circular cross-section such that the convex shape of the contact member 270 resembles, for example, a hemisphere. The sealing portion generated by the contact member 270 can circumferentially surround the injector 230 to form a hemispherical substantially fluid-tight seal with the contact surface of the eye. Alternatively, in some embodiments the contact member 270 can have a conical, or a parabolic shape. In use, a medical professional and/or a user can apply pressure to the delivery device 200 against the contact surface of the eye of the patent and/or user to generate a fluid-tight seal with the contact surface of the eye.

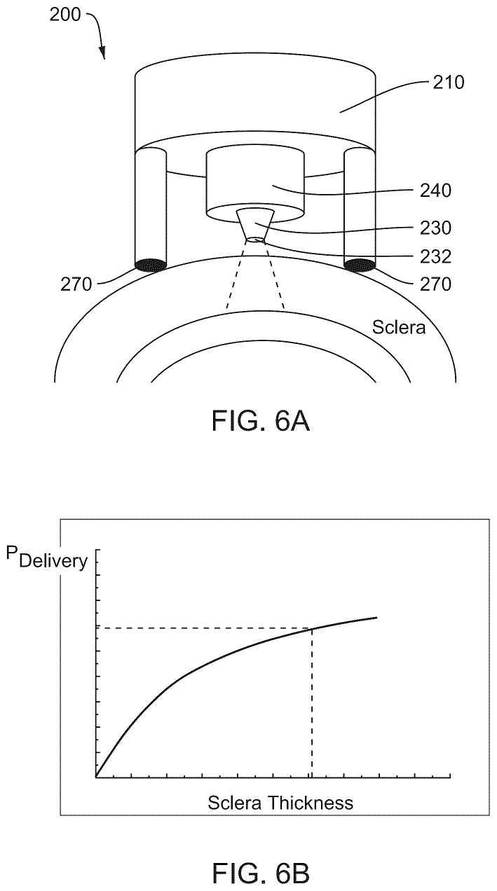

FIG. 7 illustrates a medicament delivery device 300 according to an embodiment. The medicament delivery device 300 (also referred to herein as the “delivery device”) can be similar in form and/or function to the delivery device 100 and 200, described above. For example, as described above with reference to the delivery device 100, the delivery device 300 can be used to delivery one or more medicaments and/or medicinal components to specific portions and/or regions of a human eye without using a piercing member such as a needle, cannula, stylet, and/or catheter (e.g., needle-less medicament delivery). The delivery device 300 can include a housing 310, a medicament container (not shown), an injector 330, a sensor 340, and a control unit (not shown). In some implementations, portions and/or aspects of the delivery device 300 can be similar to and/or substantially the same as portions and/or aspects of the delivery device 100 described above with reference to FIG. 5. Accordingly, such similar portions and/or aspects may not be described in further detail herein

The housing 310 defines an internal volume, compartment, and/or chamber for accommodating and/or coupling the various components of the delivery device 300. The housing 310 can be an elongated member having a circular cross-sectional shape that defines an interior volume sufficient large to accommodate and/or support the components of the delivery device 300. Alternatively, in some implementations the housing 310 can be any suitable shape. In some implementations, the housing 310 can have a shape, surface features, and/or surface materials or finishes that can be configured to increase the ergonomics of the delivery device 300, which can, for example, allow a trained healthcare worker, a medical professional and/or a user to manipulate the delivery device 300 with one hand (i.e., single-handed use). The housing 310 can be made of any suitable material or materials having sufficient structural strength and rigidity, including, for example, metal, glass, ceramic, and/or polymers. In some implementations, the housing 310 can include multiple portions that can be coupled and/or assembled together to form the interior chamber and/or compartment for receiving the components of the delivery device 300. That is, in some implementations, the housing 310 can be modular. Alternatively, in other implementations, the housing 310 can be made of a monolithic structure.

FIG. 7 shows the injector 330 can be configured to (1) produce a flow of medicinal component(s) stored and/or contained in the medicament compartment of the delivery device 300, (2) direct and/or guide the produced flow of medicinal component(s) to the nozzle 332, and (3) generate, with the aid of the nozzle 332, a stream 334 of medicinal component(s) having one or more predetermined characteristics including: a width, a cross sectional area, a cross sectional shape, a velocity of the medicinal component(s), and/or a specific trajectory that transports the medicinal component(s) to a target region and/or portion of an eye of a subject and/or patient, such as the suprachoroidal space (SCS).

The injector 330 can be any suitable injector including a high-pressure chamber, a Lorentz motor, and/or a Lorentz driver and/or actuator. The features and/or characteristics of the injector 330 can be substantially similar to the o features and/or characteristics of the injector 130 described above with reference to the delivery device 100. Consequently, the injector 330 will not be further described herein.

FIG. 7 shows the sensor 340 can be disposed and/or coupled to an outer surface of the housing 310. The sensor 340 can be one or more ultrasonic sensors disposed on a distal portion of the housing 310 such that the sensors 340 can contact an external surface of an eye of a subject and/or patient. The ultrasonic sensors 340 can be configured to emit an excitation sound wave directed towards the eye of the subject and/or patient, and detect and/or sense response sound wave produced in response to the excitation sound waves. The ultrasonic sensor 340 can include a transmitter and a receiver (not shown). The transmitter can include a piezoelectric crystal configured to emit excitation sound waves directed to the eye of the subject and/or patient. The receiver can encounter and/or detect response sound waves produced in response to the excitation sound waves after the excitation sound waves have travelled from the transmitter to a target region or portion of the eye, such as the sclera tissue, and then back to the ultrasonic sensor 340. The ultrasonic sensor 340 can measure a distance between the ultrasonic sensor an object such as the interface between the sclera and choroid (e.g., the suprachoroidal space SCS), or the choroid and the retina (or an interface therebetween), by measuring the time that passes between the emission of the excitation sound wave by the transmitter and the detection of the response sound wave by the receiver. In use, the ultrasonic sensor 340 can receive a set of instructions and/or commands from the control unit of the delivery device 300 to emit an excitation sound wave. The ultrasonic sensor 340 can direct the excitation sound wave to a portion and/or region of the eye of the subject and/or patient. The excitation sound wave can be reflected by the portion and/or region of the eye of the subject and/or patient. The reflected sound wave can be detected by the receiver. The ultrasonic sensor 340 can then determine one or more properties of the portion and/or region of the eye of the subject and/or patient. For example, in some implementations the response from the ultrasonic sensor 340 can be used to determine a thickness, density, and/or permeability of one or more layers and/or tissues of the eye of the subject and/or patient.

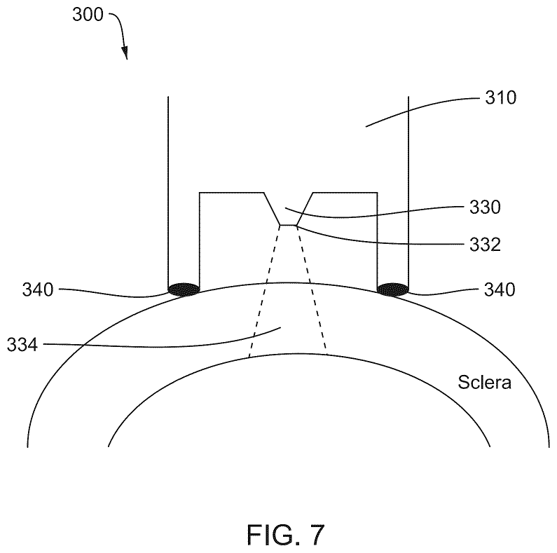

FIG. 8 illustrates a medicament delivery device 400 according to an embodiment. The medicament delivery device 400 (also referred to herein as the “delivery device”) can be similar in form and/or function to the delivery device 100, 200, and 300 described above. For example, as described above with reference to the delivery device 100, the delivery device 400 can be used to delivery one or more medicaments and/or medicinal components to specific portions and/or regions of a human eye without using a piercing member such as a needle, cannula, stylet, and/or catheter (e.g., needle-less medicament delivery). The delivery device 400 can include a housing 410, a medicament container (not shown), an injector 430, a control unit (not shown), and a contact member 470. In some implementations, portions and/or aspects of the delivery device 400 can be similar to and/or substantially the same as portions and/or aspects of the delivery device 100 described above with reference to FIG. 5. Accordingly, such similar portions and/or aspects may not be described in further detail herein

The housing 410 defines an internal volume, compartment, and/or chamber for accommodating and/or coupling the various components of the delivery device 400. The housing 410 can be an elongated member having a circular cross-sectional shape that defines an interior volume sufficient large to accommodate and/or support the components of the delivery device 400. Alternatively, in some implementations the housing 410 can be any suitable shape. In some implementations, the housing 410 can have a shape, surface features, and/or surface materials or finishes that can be configured to increase the ergonomics of the delivery device 400, which can, for example, allow a trained healthcare worker, a medical professional and/or a user to manipulate the delivery device 400 with one hand (i.e., single-handed use). The housing 410 can be made of any suitable material or materials having sufficient structural strength and rigidity, including, for example, metal, glass, ceramic, and/or polymers. In some implementations, the housing 410 can include multiple portions that can be coupled and/or assembled together to form the interior chamber and/or compartment for receiving the components of the delivery device 400. That is, in some implementations, the housing 410 can be modular. Alternatively, in other implementations, the housing 410 can be made of a monolithic structure

The injector 430 can be configured to (1) produce a flow of medicinal component(s) stored and/or contained in the medicament compartment of the delivery device 400, (2) direct and/or guide the produced flow of medicinal component(s) to a nozzle (not shown), and (3) generate, with the aid of the nozzle, a stream 434 of medicinal component(s) having one or more predetermined characteristics including: a width, a cross sectional area, a cross sectional shape, a velocity of the medicinal component(s), and/or a specific trajectory that transports the medicinal component(s) to a target region and/or portion of an eye of a subject and/or patient, such as the suprachoroidal space (SCS).

The injector 330 can be any suitable injector including a high-pressure chamber, a Lorentz motor, and/or a Lorentz driver and/or actuator. The features and/or characteristics of the injector 330 can be substantially similar to the o features and/or characteristics of the injector 130 described above with reference to the delivery device 100. Consequently, the injector 330 will not be further described herein. The injector 430 can be any suitable injector including a high-pressure chamber, a Lorentz motor, and/or a Lorentz driver and/or actuator. The features and/or characteristics of the injector 430 can be substantially similar to the o features and/or characteristics of the injector 130 described above with reference to the delivery device 100. Consequently, the injector 430 will not be further described herein.

FIG. 8 shows the delivery device 400 can include a contact member 470. The contact member 470 can be a separate structure that can be removably coupled to the delivery device 400. The contact member 470 can have any suitable shape and/or form. For example, as shown in FIG. 8, the contact member 470 can be an annular section having an inner diameter larger than the diameter of the housing 410. The contact member 470 can also include a shoulder 472 that can facilitate coupling the delivery device 400 and the contact member 470. In use, the device 400 and the contact member 470 can be coupled by introducing a distal portion of the delivery device 400 into the annular section of the contact member 470 until the distal portion of the delivery device 400 touches and or contacts the shoulders 472. In some implementations, the contact member 470 can have a predetermined distance and/or height H, measured from the distal end-portion of the contact member 470 to the position of the shoulders 472. The distance and/or height H can be selected to provide a suitable distance separating the injector 430 from the intended target region of the eye of the subject and/or patient, to ensure that the stream 434 can reach the intended target region. In some implementations, a contact member having a predetermined distance and/or height H can be selected when one or more properties of the eye of the subject care known (e.g., a thickness of the sclera tissue of the eye, a permeability of the eye, etc).

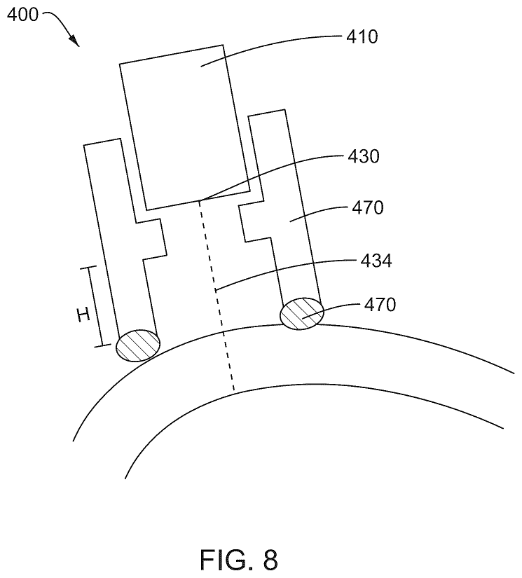

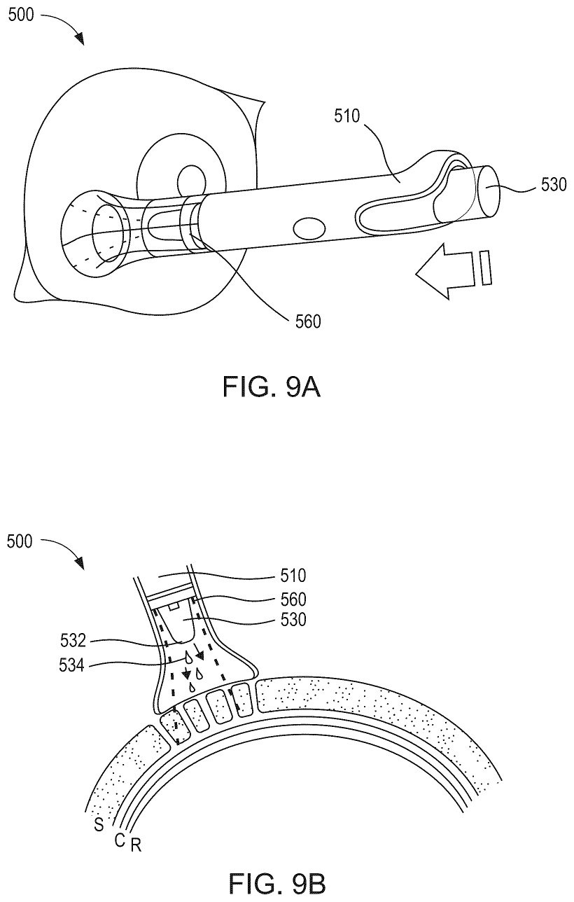

FIGS. 9A-9B illustrate a medicament delivery device 500 according to an embodiment. The medicament delivery device 500 (also referred to herein as the “delivery device”) can be similar in form and/or function to the delivery device 100, 200, 300, and 400 described above. For example, as described above with reference to the delivery device 100, the delivery device 500 can be used to delivery one or more medicaments and/or medicinal components to specific portions and/or regions of a human eye without using a piercing member such as a needle, cannula, stylet, and/or catheter (e.g., needle-less medicament delivery). The delivery device 500 can include a housing 510, a medicament container (not shown), an injector 530, a control unit (not shown), and a tissue manipulator 560. In some implementations, portions and/or aspects of the delivery device 500 can be similar to and/or substantially the same as portions and/or aspects of the delivery device 100 described above with reference to FIG. 5. Accordingly, such similar portions and/or aspects may not be described in further detail herein

The housing 510 defines an internal volume, compartment, and/or chamber for accommodating and/or coupling the various components of the delivery device 500. The housing 510 can be an elongated member having a circular cross-sectional shape that defines an interior volume sufficient large to accommodate and/or support the components of the delivery device 500. Alternatively, in some implementations the housing 510 can be any suitable shape. In some implementations, the housing 510 can have a shape, surface features, and/or surface materials or finishes that can be configured to increase the ergonomics of the delivery device 500, which can, for example, allow a trained healthcare worker, a medical professional and/or a user to manipulate the delivery device 500 with one hand (i.e., single-handed use). The housing 510 can be made of any suitable material or materials having sufficient structural strength and rigidity, including, for example, metal, glass, ceramic, and/or polymers. In some implementations, the housing 510 can include multiple portions that can be coupled and/or assembled together to form the interior chamber and/or compartment for receiving the components of the delivery device 500. That is, in some implementations, the housing 510 can be modular. Alternatively, in other implementations, the housing 510 can be made of a monolithic structure

FIGS. 9A-9B show the injector 530 can be configured to (1) produce a flow of medicinal component(s) stored and/or contained in the medicament compartment of the delivery device 500, (2) direct and/or guide the produced flow of medicinal component(s) to a nozzle 532, and (3) generate, with the aid of the nozzle 532, a stream 534 of medicinal component(s) to transports the medicinal component(s) to a target region and/or portion of an eye of a subject and/or patient. The injector 530 can be any suitable injector including a high-pressure chamber, a Lorentz motor, and/or a Lorentz driver and/or actuator. The features and/or characteristics of the injector 530 can be substantially similar to the o features and/or characteristics of the injector 130 described above with reference to the delivery device 100. Consequently, the injector 530 will not be further described herein.

FIG. 9B shows the delivery device 500 includes a tissue manipulator 560. The tissue manipulator 560 can be a thermal ablation device configured to heat a small area of an ocular tissue layer of an eye of a subject and/or patient to deplete the ocular tissue layer at a that site without significantly damaging ocular tissue disposed underneath the exposed area. In some implementations the tissue manipulator 560 can include a laser gun. The tissue manipulator 560 can be configured to deposit optical energy over a small and/or controlled area of an ocular tissue (e.g., the sclera), causing evaporation of water and formation of microchannels within the sclera that can facilitate the diffusion of a stream of medicinal component(s), as shown in FIG. 9B. In some implementations the tissue manipulation component 560 can be operatively coupled to the control unit of the delivery device 500 to activate, and/or control the operation of the tissue manipulator 560. In such implementations, the tissue manipulator 560 can receive one or more electrical signals from the control unit of the delivery device 500 associated with a set of instructions to adjust one or more parameter of the tissue manipulator 560 such as a wavelength, a frequency, a pulse length, a pulse energy, a number of pulses, an exposure time (e.g., the duration of an interaction), and/or a pulse repetition.

In one embodiment, an angiogenesis inhibitor is delivered via the methods and devices described herein, in one embodiment, is interferon gamma 1β, interferon gamma 1β (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, Astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260 or XV615.

In one embodiment, a VEGF modulator is delivered via one of the devices described herein. In one embodiment, the VEGF modulator is a VEGF antagonist. In one embodiment, the VEGF modulator is a VEGF-receptor kinase antagonist, an anti-VEGF antibody or fragment thereof, an anti-VEGF receptor antibody, an anti-VEGF aptamer, a small molecule VEGF antagonist, a thiazolidinedione, a quinoline or a designed ankyrin repeat protein (DARPin). In one embodiment, the VEGF antagonist is an antagonist of a VEGF receptor (VEGFR), i.e., a drug that inhibits, reduces, or modulates the signaling and/or activity of a VEGFR. The VEGFR may be a membrane-bound or soluble VEGFR. In a further embodiment, the VEGFR is VEGFR-1, VEGFR-2 or VEGFR-3. In one embodiment, the VEGF antagonist targets the VEGF-C protein. In another embodiment, the VEGF modulator is an antagonist of a tyrosine kinase or a tyrosine kinase receptor. In another embodiment, the VEGF modulator is a modulator of the VEGF-A protein. In yet another embodiment, the VEGF antagonist is a monoclonal antibody. In a further embodiment, the monoclonal antibody is a humanized monoclonal antibody.

In one embodiment, the VEGF modulator is one or more of the following: AL8326, 2C3 antibody, AT001 antibody, HyBEV, bevacizumab (Avastin®), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, sunitinib malate (Sutent®), INDUS815C, R84 antibody, KD019, NM3, allogenic mesenchymal precursor cells combined with an anti-VEGF antagonist (e.g., anti-VEGF antibody), MGCD265, MG516, VEGF-Receptor kinase inhibitor, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Fltl receptor, cediranib (Recentin™), AV-951, tivozanib (KRN-951), regorafenib (Stivarga®), volasertib (BI6727), CEP11981, KH903, lenvatinib (E7080), lenvatinib mesylate, terameprocol (EM1421), ranibizumab (Lucentis®), pazopanib hydrochloride (Votrient™), PF00337210, PRS050, SP01 (curcumin), carboxyamidotriazole orotate, hydroxychloroquine, linifanib (ABT869, RG3635), fluocinolone acetonide (Iluvien®), ALG1001, AGN150998, DARPin MP0112, AMG386, ponatinib (AP24534), AVA101, nintedanib (Vargatef™), BMS690514, KH902, golvatinib (E7050), everolimus (Afinitor®), dovitinib lactate (TKI258, CHIR258), ORA101, ORA102, axitinib (Inlyta®, AG013736), plitidepsin (Aplidin®), PTC299, aflibercept (Zaltrap®, Eylea®), pegaptanib sodium (Macugen™, LI900015), verteporfin (Visudyne®), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPL010, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PTI101, TG100948, ICS283, XL647, enzastaurin hydrochloride (LY317615), BC194, quinolines, COT601M06.1, COT604M06.2, Mabion VEGF, SIR-Spheres coupled to anti-VEGF or VEGF-R antibody, Apatinib (YN968D1), and AL3818. In addition, delivery of a VEGF antagonist using the devices and non-surgical methods disclosed herein may be combined with one or more agents listed herein or with other agents known in the art, either in a single or multiple formulations.

In one embodiment, an immunosuppressive agent is delivered via one of the devices described herein. In a further embodiment, the immunosuppressive agent is a glucocorticoid, cytokine inhibitor, cytostatic, alkylating agent, anti-metabolite, folic acid analogue, cytotoxic antibiotic, interferon, opioid, T-cell receptor directed antibody or an IL-2 receptor directed antibody. In one embodiment, the immunosuppressive agent is an anti-metabolite and the anti-metabolite is a purine analog, pyrimidine analogue, folic acid analogue or a protein synthesis inhibitor. In another embodiment, the immunosuppressive agent is an interleukin-2 inhibitor (e.g., basiliximab or daclizumab). Other immunosuppressive agents amenable for use with the methods and formulations described herein include, but are not limited to cyclophosphamide, nitrosourea, methotrexate, azathioprine, mercaptopurine, fluorouracil, dactinomycin, anthracycline, mitomycin C, bleomycin, mithramycin, muromonab-CD3, cyclosporine, tacrolimus, sirolimus or mycophenolate. In one embodiment, the drug formulation comprises an effective amount mycophenolate.

In one embodiment, the drug formulation delivered via one of the devices described herein comprises an effective amount of vascular permeability inhibitor. In one embodiment, the vascular permeability inhibitor is a vascular endothelial growth factor (VEGF) antagonist or an angiotensin converting enzyme (ACE) inhibitor. In a further embodiment, the vascular permeability inhibitor is an angiotensin converting enzyme (ACE) inhibitor and the ACE inhibitor is captopril.

In one embodiment, the drug is a steroid or a non-steroid anti-inflammatory drug (NSAID). In another embodiment, the anti-inflammatory drug is an antibody or fragment thereof, an anti-inflammatory peptide(s) or an anti-inflammatory aptamer(s).

Steroidal compounds that can be administered via the methods provided herein include hydrocortisone, hydrocortisone-17-butyrate, hydrocortisone-17-aceponate, hydrocortisone-17-buteprate, cortisone, tixocortol pivalate, prednisolone, methylprednisolone, prednisone, triamcinolone, triamcinolone acetonide, mometasone, amcinonide, budesonide, desonide, fluocinonide, halcinonide, bethamethasone, bethamethasone dipropionate, dexamethasone, fluocortolone, hydrocortisone-17-valerate, halometasone, alclometasone dipropionate, prednicarbate, clobetasone-17-butyrate, clobetasol-17-propionate, fluocortolone caproate, fluocortolone pivalate, fluprednidene acetate and prednicarbate.

Specific classes of NSAIDs that can be administered via the methods provided herein include salicylates, propionic acid derivatives, acetic acid derivatives, enolic acid derivatives, fenamic acid derivatives and cyclooxygenase-2 (COX-2) inhibitors. In one embodiment, the devices and methods provided herein are used to deliver one or more of the following NSAIDs to the eye of a patient in need thereof: acetylsalicylic acid, diflunisal, salsalate, ibuprofen, dexibuprofen, naproxen, fenoprofen, keotoprofen, dexketoprofen, flurbiprofen, oxaprozin, loxaprofen, indomethacin, tolmetin, sulindac, etodolac, ketorolac, diclofenac or nabumetone, piroxicam, meloxicam, tenoxicam, droxicam, lornoxicam or isoxicam, mefanamic acid, meclofenamic acid, flufenamic acid, tolfenamic acid, celecoxib, refecoxib, valdecoxib, parecoxib, lumiracoxib, etoricoxib or firocoxib.

Other examples of anti-inflammatory drugs include, but are not limited to: mycophenolate, remicase, nepafenac, 19AV agonist(s), 19GJ agonists, 2MD analogs, 4SC101, 4SC102, 57-57, 5-HT2 receptor antagonist, 64G12, A804598, A967079, AAD2004, AB1010, AB224050, abatacept, etaracizumab (Abegrin™), Abevac®, AbGn134, AbGn168, Abki, ABN912, ABR215062, ABR224050, cyclosporine (Abrammune®), docosanol (behenyl alcohol, Abreva®), ABS15, ABS4, ABS6, ABT122, ABT325, ABT494, ABT874, ABT963, ABXIL8, ABXRB2, AC430, Accenetra, lysozyme chloride (Acdeam®), ACE772, aceclofenac (Acebloc, Acebid, Acenac), acetaminophen, chlorzoxazone, serrapeptase, tizanidine hydrochloride, betadex, Aceclogesic Plus, Aceclon, Acecloren, Aceclorism, acecrona, Aceffein, acemetacin, asprin (Acenterine), Acetal-SP (Aceclofenac-combination, ibuprofen, Acetyl-G, acetylsalicylate dl-lysine, acetylsalicylic acid, Acicot, Acifine, Acik, Aclocen, Acloflam-P, Aclomore, Aclon, A-CQ, ACS15, actarit, Actemra, Acthelea liofilizado, Actifast, Actimab-B, Actiquim, Actirin, Actis PLUS, activated leukocyte cell adhesion molecule antibody, Acular X, AD452, adalimumab, ADAMTS5 inhibitor, ADC1001, Adco-Diclofenac, Adco-Indomethacin, Adco-Meloxicam, Adco-Naproxen, Adco-Piroxicam, Adcort, Adco-Sulindac, adenosine triphosphate disodium, AdenosineA2a Receptor Agonist, Adimod, Adinos, Adioct, Adiodol, Adipoplus, adipose derived stem and/or regenerative cells, Adizen, Adpep, Advacan, Advagraf, Advel, Adwiflam, AEB071, Aental, Afenac, Affen Plus, Afiancen, Afinitor, Aflamin, Aflazacort, Aflogen, Afloxan, AFM15, AFM16, AFM17, AFM23, Afpred-Dexa, AFX200, AG011, Agafen, aganirsen, AGI1096, Agidex, AGS010, Agudol, A-Hydrocort, AIK1, AlN457, Airtal, AIT110, AJM300, ajulemic acid, AK106, AL-24-2A1, AL4-1A1, Ala Cort, Alanz, Albumin immune-globulin, alclometasone dipropionate, ALD518, aldesleukin, Aldoderma, alefacept, alemtuzumab, Alequel™, Alergolon, Alergosone, Aletraxon, Alfenac, Algason, Algin vek coat, Algioflex, Algirex, Algivin Plus, alicaforsen sodium, Alin, Alinia, Aliviodol, Aliviosin, alkaline phosphatase, ALKS6931, allantoin, Allbupen, Allmol, Allochrysine, allogeneic endothelial cells, allogeneic mesenchymal precursor cells, allogeneic mesenchymal stem cells, alminoprofen, alpha 1 antitrypsin, Alpha 7 nicotinic agonists, alpha amylase, alpha chymotrypsin, alpha fetoprotein, alpha linolenic acid, alpha-1-antitrypsin, α2β1 integrin inhibitor, α2β1 integrin inhibitor, αvβ3 inhibitor, αvβ5 inhibitor, αvβ1 inhibitor, α4β1 inhibitor, α4β7 inhibitor Alphacort, Alphafen, alpha-hexidine, alpha-trypsin, Alphintern, Alpinamed mobility omega 3, Alpoxen, AL-Rev1, Alterase, ALX0061, ALX0761, ALXN1007, ALXN1102, AM3840, AM3876, AMAB, AMAP102, Amason, Ambene, AmbezimG, amcinonide, AME133v, Amecin, Ameloteks, A-Methapred, Amevive, AMG108, AMG139, AMG162, AMG181, AMG191, AMG220, AMG623, AMG674, AMG714, AMG719, AMG729, AMG827, Amidol, amifampridine phosphate, diclofenac (Emifenac®), Amimethacin, amiprilose hydrochloride, Amiprofen, Ammophos, Amoflam, AMP110, Ampikyy, Ampion, ampiroxicam, amtolmetin guacil, AMX256, AN6415, ANA004, ANA506, Anabu, Anacen, Anaflam, Anaflex ACI, Anaida, anakinra, Analgen Artritis, Anapan, Anaprox, Anavan, Anax, Anco, andrographis, Aneol, Anergix, Anervax.RA™ (therapeutic peptide vaccine), Anflene, ANG797, Anilixin, Anmerushin, Annexin 1 peptides, annexin A5, Anodyne, Ansaid, Anspirin, Antarene, anti BST2 antibody, anti C5a MAb, anti ILT7 antibody, anti VLAl antibody, anti-alphall antibody, anti-CD4 802-2, anti-CD86 monoclonal antibody, anti-chemokine, anti-DC-SIGN, anti-HMGB-1 MAb, anti-IL-18 Mab, anti-IL-1R MAb, anti-IL-1R MAb, anti-IL23 BRISTOL, anti-interleukin-1β antibody, anti-LIGHT antibody, anti-MIF antibody, anti-MIF antibody, anti-miR181a, antioxidant inflammation modulators, Antiphlamine, AntiRAGE MAb, antithrombin III, Anti-TIRC-7 MAb, Anusol-HC, Anyfen, AP105, AP1089, AP1189, AP401, AP501, apazone, APD334, Apentac, APG103, Apidone, apilimod mesylate, Apitac, Apitoxin, Apizel, APN inhibitor, apo-azathioprine, Apo-dexamethasone, ApoE mimetics, ApoFasL, apo-Indomethacin, apo-mefenamic, apo-methotrexate, apo-nabumetone, Apo-Napro-NA, apo-Naproxen, aponidin, apo-Phenylbutazone, apo-Piroxicam, apo-Sulin, Apo-Tenoxicam, apo-Tiaprofenic, Apranax, apremilast, apricoxib, Aprofen, Aprose, Aproxen, APX001 antibody, APX007 antibody, APY0201, AqvoDex, AQX108, AQX1125, AQX131135, AQX140, AQX150, AQX200, AQX356, AQXMN100, AQXMN106, ARA290, Arava, Arcalyst, Arcoxia, Arechin, Arflur, ARG098, ARG301, arginine aescin, arginine deiminase (pegylated), ARGX109 antibody, ARGX110, Arheuma, Aristocort, Aristospan, Ark-AP, ARN4026, Arofen, Aroff EZ, Arolef, Arotal, Arpibru, Arpimune, Arpu Shuangxin, ARQ101, Arrestin SP, Arrox, ARRY162, ARRY371797, ARRY614, ARRY872, ART621, Artamin, Arthfree, Artho Tech, Arthrexin, Arthrispray, Arthrotec, aeterna shark cartilage extract (Arthrovas™, Neoretna™, Psovascar™), Artifit, Artigo, Artin, Artinor, Artisid, Artoflex, Artren Hipergel, Artridol, Artrilase, Artrocaptin, Artrodiet, Artrofen, Artropan, Artrosil, Artrosilene, Artrotin, Artrox, Artyflam, Arzerra, AS604850, AS605858, Asacol, ASA-Grindeks, Asazipam, Aseclo, ASF1096, ASF1096, ASK8007, ASKP1240, ASLAN003, Asmo ID, Asonep, ASP015K, ASP2408, ASP2409, Aspagin, Aspeol, Aspicam, Aspirimex, AST120, astaxanthin, AstroCort, Aszes, AT002 antibody, AT007, AT008 antibody, AT008 antibody, AT010, AT1001, atacicept, Ataspin, Atepadene, Atgam, ATG-Fresenius, Athrofen, ATI003, atiprimod, ATL1222, ATN103, ATN192, ATR107, Atri, Atrmin, Atrosab antibody, ATX3105, AU801, auranofin, Aurobin, Auropan, Aurothio, aurotioprol, autologous adipose derived regenerative cells, Autonec, Avandia, AVE9897, AVE9940, Avelox, Avent, AVI3378, Avloquin, AVP13546, AVP13748, AVP28225, AVX002, Axcel Diclofenac, Axcel Papain, Axen, AZ17, AZ175, Azacortid, AZA-DR, Azafrine, Azamun, Azanin, Azap, Azapin, Azapren, Azaprin, Azaram, Azasan, azathioprine, AZD0275, AZD0902, AZD2315, AZD5672, AZD6703, AZD7140, AZD8309, AZD8566, AZD9056, Azet, Azintrel, azithromycin, Az-od, Azofit, Azolid, Azoran, Azulene, Azulfidine, Azulfin, B1 antagonists, Baclonet, BAF312, BAFF Inhibitor, Bages, Baily S.P., Baleston, Balsolone, baminercept alfa, bardoxolone methyl, baricitinib, Barotase, Basecam, basiliximab, Baxmune, Baxo, BAY869766, BB2827, BCX34, BCX4208, Becfine, Beclate-C, Beclate-N, Beclolab Q, beclomethasone dipropionate, Beclorhin, Becmet-CG, Begita, Begti, belatacept, belimumab, Belosalic, Bemetson, Ben, Benevat, Benexam, Benflogin, Benisan, Benlysta, Benlysta, benorilate, Benoson, benoxaprofen, Bentol, benzydamine hydrochloride, Benzymin, Beofenac, Berafen, Berinert, Berlofen, Bertanel, Bestamine, Bestofen, Beta Nicip, Betacort, Betacorten G, Betafoam, beta-glucan, Betalar, Beta-M, Betamed, Betamesol, betamethasone, betamethasone dipropionate, betamethasone sodium, betamethasone sodium phosphate, betamethasone valerate, Betane, Betanex, Betapanthen, Betapar, Betapred, Betason, Betasonate, Betasone, Betatrinta, Betaval, Betazon, Betazone, Betesil, Betnecort, Betnesol, Betnovate, Bextra, BFPC13, BFPC18, BFPC21, BFPT6864, BG12, BG9924, BI695500, BI695501, BIA12, Big-Joint-D, BIIB023 antibody, Bi-ksikam, Bingo, BioBee, Bio-Cartilage, Bio-C-Sinkki, Biodexone, Biofenac, Bioreucam, Biosone, Biosporin, BIRB796, Bitnoval, Bitvio, Bivigam, BKT140, BKTP46, BL2030, BL3030, BL4020, BL6040, BL7060, BLI1300, blisibimod, Blokium B12, Blokium Gesic, Blokium, BMS066, BMS345541, BMS470539, BMS561392, BMS566419, BMS582949, BMS587101, BMS817399, BMS936557, BMS945429, BMS-A, BN006, BN007, BNP166, Bonacort, Bonas, bone marrow stromal cell antigen 2 antibody, Bonflex, Bonifen, Boomiq, Borbit, Bosong, BR02001, BR3-FC, Bradykinin B1 Receptor Antagonist, Bredinin, Brexecam, Brexin, Brexodin, briakinumab, Brimani, briobacept, Bristaflam, Britten, Broben, brodalumab, Broen-C, bromelains, Bromelin, Bronax, Bropain, Brosiral, Bruace, Brufadol, Brufen, Brugel, Brukil, Brusil, BT061, BTI9, BTK kinase inhibitors, BTT1023 antibody, BTT1507, bucillamine, Bucillate, Buco Reigis, bucolome, Budenofalk, budesonide, Budex, Bufect, Bufencon, Bukwang Ketoprofen, Bunide, Bunofen, Busilvex, busulfan, Busulfex, Busulipo, Butartrol, Butarut B12, Butasona, Butazolidin, Butesone, Butidiona, BVX10, BXL628, BYM338, B-Zone, Cl esterase inhibitor, C243, c4462, c5997, C5aQb, c7198, c9101, C9709, c9787, CAB101, cadherin 11 antibody, caerulomycin A, CAL263, Calcort, Calmatel, CAM3001, Camelid Antibodies, Camlox, Camola, Campath, Camrox, Camtenam, canakinumab, Candida albicans antigen, Candin, cannabidiol, CAP1.1, CAP1.2, CAP2.1, CAP2.2, CAP3.1, CAP3.2, Careram, Carimune, Cariodent, Cartifix, CartiJoint, Cartilago, Cartisafe-DN, Cartishine, Cartivit, Cartril-S, Carudol, CaspaCIDe, CaspaCIDe, Casyn, CAT1004, CAT1902, CAT2200, Cataflam, Cathepsin S inhibitor, Catlep, CB0114, CB2 agonist, CC0478765, CC10004, CC10015, CC1088, CC11050, CC13097, CC15965, CC16057, CC220, CC292, CC401, CC5048, CC509, CC7085, CC930, CCR1 antagonist, CCR6 inhibitor, CCR7 antagonist, CCRL2 antagonist, CCX025, CCX354, CCX634, CD Diclofenac, CD102, CD103 antibody, CD103 antibody, CD137 antibody, CD16 antibody, CD18 antibody, CD19 antibody, CD1d antibody, CD20 antibody, CD200Fc, CD209 antibody, CD24, CD3 antibody, CD30 antibody, CD32A antibody, CD32B antibody, CD4 antibody, CD40 ligand, CD44 antibody, CD64 antibody, CDC839, CDC998, CDIM4, CDIM9, CDK9-Inhibitor, CDP146, CDP323, CDP484, CDP6038, CDP870, CDX1135, CDX301, CE224535, Ceanel, Cebedex, Cebutid, Ceclonac, Ceex, CEL2000, Celact, Celbexx, Celcox, Celebiox, Celebrex, Celebrin, Celecox, celecoxib, Celedol, Celestone, Celevex, Celex, CELG4, Cell adhesion molecule antagonists, CellCept, Cellmune, Celosti, Celoxib, Celprot, Celudex, cenicriviroc mesylate, cenplacel-l, CEP11004, CEP37247, CEP37248, Cephyr, Ceprofen, Certican, certolizumab pegol, Cetofenid, Cetoprofeno, cetylpyridinium chloride, CF101, CF402, CF502, CG57008, CGEN15001, CGEN15021, CGEN15051, CGEN15091, CGEN25017, CGEN25068, CGEN40, CGEN54, CGEN768, CGEN855, CGI1746, CGI560, CGI676, Cgtx-Peptides, CH1504, CH4051, CH4446, chaperonin 10, chemokine C-C motif ligand 2, chemokine C-C motif ligand 2 antibody, chemokine C-C motif ligand 5 antibody, chemokine C-C motif receptor 2 antibody, chemokine C-C motif receptor 4 antibody, chemokine C-X-C motif ligand 10 antibody, chemokine C-X-C motif ligand 12 aptamer, Chemotaxis Inhibitor, Chillmetacin, chitinase 3-like 1, Chlocodemin, Chloquin, chlorhexidine gluconate, chloroquine phosphate, choline magnesium trisalicylate, chondroitin sulfate, Chondroscart, CHR3620, CHR4432, CHR5154, Chrysalin, Chuanxinlian, Chymapra, Chymotase, chymotrypsin, Chytmutrip, CI202, CI302, Cicloderm-C, Ciclopren, Cicporal, Cilamin, Cimzia, cinchophen, cinmetacin, cinnoxicam, Cinoderm, Cinolone-S, Cinryze, Cipcorlin, cipemastat, Cipol-N, Cipridanol, Cipzen, Citax F, Citogan, Citoken T, Civamide, CJ042794, CJ14877, c-Kit monoclonal antibody, cladribine, Clafen, Clanza, Claversal, clazakizumab, Clearoid, Clease, Clevegen, Clevian, Clidol, Clindac, Clinoril, Cliptol, Clobenate, Clobequad, clobetasol butyrate, clobetasol propionate, Clodol, clofarabine, Clofen, Clofenal LP, Clolar, Clonac, Clongamma, clonixin lysine, Clotasoce, Clovacort, Clovana, Cloxin, CLT001, CLT008, C-MAF Inhibitor, CMPX1023, Cnac, CNDO201, CNI1493, CNTO136, CNTO148, CNTO1959, Cobefen, CoBenCoDerm, Cobix, Cofenac, Cofenac, COG241, COL179, colchicine, Colchicum Dispert, Colchimax, Colcibra, Coledes A, Colesol, Colifoam, Colirest, collagen, type V, Comcort, complement component (3b/4b) receptor 1, Complement Component Cls Inhibitors, complement component C3, complement factor 5a receptor antibody, complement factor 5a receptor antibody, complement factor D antibody, Condrosulf, Condrotec, Condrothin, conestat alfa, connective tissue growth factor antibody, Coolpan, Copaxone, Copiron, Cordefla, Corhydron, Cort S, Cortan, Cortate, Cort-Dome, Cortecetine, Cortef, Corteroid, Corticap, Corticas, Cortic-DS, corticotropin, Cortiderm, Cortidex, Cortiflam, Cortinet M, Cortinil, Cortipyren B, Cortiran, Cortis, Cortisolu, cortisone acetate, Cortival, Cortone acetate, Cortopin, Cortoral, Cortril, Cortypiren, Cosamine, Cosone, cosyntropin, COT Kinase Inhibitor, Cotilam, Cotrisone, Cotson, Covox, Cox B, COX-2/5-LO Inhibitors, Coxeton, Coxflam, Coxicam, Coxitor, Coxtral, Coxypar, CP195543, CP412245, CP424174, CP461, CP629933, CP690550, CP751871, CPSI2364, C-quin, CR039, CR074, CR106, CRA102, CRAC channel inhibitor, CRACM ion channel inhibitor, Cratisone, CRB15, CRC4273, CRC4342, C-reactive protein 2-methoxyethyl phosphorothioate oligonucleotide, CreaVax-RA, CRH modulators, critic-aid, Crocam, Crohnsvax, Cromoglycic acid, cromolyn sodium, Cronocorteroid, Cronodicasone, CRTX803, CRx119, CRx139, CRx150, CS502, CS670, CS706, CSFIR Kinase Inhibitors, CSL324, CSL718, CSL742, CT112, CT1501R, CT200, CT2008, CT2009, CT3, CT335, CT340, CT5357, CT637, CTP05, CTP10, CT-P13, CTP17, Cuprenil, Cuprimine, Cuprindo, Cupripen, Curaquin, Cutfen, CWF0808, CWP271, CX1020, CX1030, CX1040, CX5011, Cx611, Cx621, Cx911, CXC chemokine receptor 4 antibody, CXCL13 antibodies, CXCR3 antagonists, CXCR4 antagonist, Cyathus 1104 B, Cyclo-2, Cyclocort, cyclooxygenase-2 inhibitor, cyclophosphamide, Cyclorine, Cyclosporin A Prodrug, Cyclosporin analogue A, cyclosporine, Cyrevia, Cyrin CLARIS, CYT007TNFQb, CYT013IL1bQb, CYT015IL17Qb, CYT020TNFQb, CYT107, CYT387, CYT99007, cytokine inhibitors, Cytopan, Cytoreg, CZC24832, D1927, D9421C, daclizumab, danazol, Danilase, Dantes, Danzen, dapsone, Dase-D, Daypro, Daypro Alta, Dayrun, Dazen, DB295, DBTP2, D-Cort, DD1, DD3, DE096, DE098, Debio0406, Debio0512, Debio0615, Debio0618, Debio1036, Decaderm, Decadrale, Decadron, Decadronal, Decalon, Decan, Decason, Decdan, Decilone, Declophen, Decopen, Decorex, Decorten, Dedema, Dedron, Deexa, Defcort, De-flam, Deflamat, Deflan, Deflanil, Deflaren, Deflaz, deflazacort, Defnac, Defnalone, Defnil, Defosalic, Defsure, Defza, Dehydrocortison, Dekort, Delagil, delcasertib, delmitide, Delphicort, Deltacorsolone prednisolone (Deltacortril), Deltafluorene, Deltasolone, Deltasone, Deltastab, Deltonin, Demarin, Demisone, Denebola, denileukin diftitox, denosumab, Denzo, Depocortin, Depo-medrol, Depomethotrexate, Depopred, Deposet, Depyrin, Derinase, Dermol, Dermolar, Dermonate, Dermosone, Dersone, Desketo, desonide, desoxycorticosterone acetate, Deswon, Dexa, Dexabene, Dexacip, Dexacort, dexacortisone, Dexacotisil, dexadic, dexadrin, Dexadron, Dexafar, Dexahil, Dexalab, Dexalaf, Dexalet, Dexalgen, dexallion, dexalocal, Dexalone, Dexa-M, Dexamecortin, Dexamed, Dexamedis, dexameral, Dexameta, dexamethasone, dexamethasone acetate, dexamethasone palmitate, dexamethasone phosphate, dexamethasone sodium metasulfobenzoate, dexamethasone sodium phosphate, Dexamine, Dexapanthen, Dexa-S, Dexason, Dexatab, Dexatopic, Dexaval, Dexaven, Dexazolidin, Dexazona, Dexazone, Dexcor, Dexibu, dexibuprofen, Dexico, Dexifen, Deximune, dexketoprofen, dexketoprofen trometamol, Dexmark, Dexomet, Dexon I, Dexonalin, Dexonex, Dexony, Dexoptifen, Dexpin, Dextan-Plus, dextran sulfate, Dezacor, Dfz, diacerein, Diannexin, Diastone, Dicarol, Dicasone, Dicknol, Diclo, Diclobon, Diclobonse, Diclobonzox, Diclofast, Diclofen, diclofenac, diclofenac beta-dimethylaminoethanol, diclofenac deanol, diclofenac diethylamine, diclofenac epolamine, diclofenac potassium, diclofenac resinate, diclofenac sodium, Diclogen AGIO, Diclogen Plus, Diclokim, Diclomed, Diclo-NA, Diclonac, Dicloramin, Dicloran, Dicloreum, Diclorism, Diclotec, Diclovit, Diclowal, Diclozem, Dico P, Dicofen, Dicoliv, Dicorsone, Dicron, Dicser, Difena, Diffutab, diflunisal, dilmapimod, Dilora, dimethyl sulfone, Dinac, D-Indomethacin, Dioxaflex Protect, Dipagesic, Dipenopen, Dipexin, Dipro AS, Diprobeta, Diprobetasone, Diproklenat, Dipromet, Dipronova, Diprosone, Diprovate, Diproxen, Disarmin, Diser, Disopain, Dispain, Dispercam, Distamine, Dizox, DLT303, DLT404, DM199, DM99, DMI9523, dnaJP1, DNX02070, DNX04042, DNX2000, DNX4000, docosanol, Docz-6, Dolamide, Dolaren, Dolchis, Dolex, Dolflam, Dolfre, Dolgit, Dolmax, Dolmina, Dolo Ketazon, Dolobest, Dolobid, Doloc, Dolocam, Dolocartigen, Dolofit, Dolokind, Dolomed, Dolonac, Dolonex, Dolotren, Dolozen, Dolquine, Dom0100, Dom0400, Dom0800, Domet, Dometon, Dominadol, Dongipap, Donica, Dontisanin, doramapimod, Dorixina Relax, Dormelox, Dorzine Plus, Doxatar, Doxtran, DP NEC, DP4577, DP50, DP6221, D-Penamine, DPIV/APN Inhibitors, DR1 Inhibitors, DR4 Inhibitors, DRA161, DRA162, Drenex, DRF4848, DRL15725, Drossadin, DSP, Duexis, Duo-Decadron, Duoflex, Duonase, DV1079, DV1179, DWJ425, DWP422, Dymol, DYN15, Dynapar, Dysmen, E5090, E6070, Easy Dayz, Ebetrexat, EBI007, EC0286, EC0565, EC0746, Ecax, Echinacea purpurea extract, EC-Naprosyn, Econac, Ecosprin 300, Ecosprin 300, Ecridoxan, eculizumab, Edecam, efalizumab, Efcortesol, Effigel, Eflagen, Efridol, EGFR Antibody, EGS21, eIF5A1 siRNA, Ekarzin, elafin, Eldoflam, Elidel, Eliflam, Elisone, Elmes, Elmetacin, ELND001, ELND004, elocalcitol, Elocom, elsibucol, Emanzen, Emcort, Emifen, Emifenac, emorfazone, Empynase, emricasan, Emtor, Enable, Enbrel, Enceid, EncorStat, Encortolon, Encorton, Endase, Endogesic, Endoxan, Enkorten, Ensera, Entocort, Enzylan, Epanova, Eparang, Epatec, Epicotil, epidermal growth factor receptor 2 antibody, epidermal growth factor receptor antibody, Epidixone, Epidron, Epiklin, EPPA1, epratuzumab, EquiO, Erac, Erazon, ERB041, ERB196, Erdon, EryDex, Escherichia coli enterotoxin B subunit, Escin, E-Selectin Antagonists, Esfenac, ESN603, esonarimod, Esprofen, estetrol, Estopein, Estrogen Receptor beta agonist, etanercept, etaracizumab, ETC001, ethanol propolis extract, ETI511, etiprednol dicloacetate, Etodin, Etodine, Etodol, etodolac, Etody, etofenamate, Etol Fort, Etolac, Etopin, etoricoxib, Etorix, Etosafe, Etova, Etozox, Etura, Eucob, Eufans, eukaryotic translation initiation factor 5A oligonucleotide, Eunac, Eurocox, Eurogesic, everolimus, Evinopon, EVT401, Exaflam, EXEL9953, Exicort, Expen, Extra Feverlet, Extrapan, Extrauma, Exudase, F16, F991, Falcam, Falcol, Falzy, Farbovil, Farcomethacin, Farnerate, Farnezone, Farnezone, Farotrin, fas antibody, Fastflam, FasTRACK, Fastum, Fauldmetro, FcgammaRIA antibody, FE301, Febrofen, Febrofid, felbinac, Feldene, Feldex, Feloran, Felxicam, Fenac, Fenacop, Fenadol, Fenaflan, Fenamic, Fenaren, Fenaton, Fenbid, fenbufen, Fengshi Gutong, Fenicort, Fenopine, fenoprofen calcium, Fenopron, Fenris, Fensupp, Fenxicam, fepradinol, Ferovisc, Feverlet, fezakinumab, FG3019, FHT401, FHTCT4, FID114657, figitumumab, Filexi, filgrastim, Fillase, Final, Findoxin, fingolimod hydrochloride, firategrast, Firdapse, Fisiodar, Fivasa, FK778, Flacoxto, Fladalgin, Flagon, Flamar, Flamcid, Flamfort, Flamide, Flaminase, Flamirex Gesic, Flanid, Flanzen, Flaren, Flaren, Flash Act, Flavonoid Anti-inflammatory Molecule, Flebogamma DIF, Flenac, Flex, Flexafen 400, Flexi, Flexidol, Flexium, Flexon, Flexono, Flogene, Flogiatrin B12, Flogomin, Flogoral, Flogosan, Flogoter, Flo-Pred, Flosteron, Flotrip Forte, Flt3 inhibitors, fluasterone, Flucam, Flucinar, fludrocortisone acetate, flufenamate aluminum, flumethasone, Flumidon, flunixin, fluocinolone, fluocinolone acetonide, fluocinonide, fluocortolone, Fluonid, fluorometholone, Flur, flurbiprofen, Fluribec, Flurometholone, Flutal, fluticasone, fluticasone propionate, Flutizone, Fluzone, FM101 antibody, fms-related tyrosine kinase 1 antibody, Folitrax, fontolizumab, formic acid, Fortecortin, Fospeg, fostamatinib disodium, FP1069, FP13XX, FPA008, FPA031, FPT025, FR104, FR167653, Framebin, Frime, Froben, Frolix, FROUNT Inhibitors, Fubifen PAP, Fucole ibuprofen, Fulamotol, Fulpen, Fungifin, Furotalgin, fusidate sodium, FX002, FX141L, FX201, FX300, FX87L, Galectin modulators, gallium maltolate, Gamimune N, Gammagard, Gamma-I.V., GammaQuin, Gamma-Venin, Gamunex, Garzen, Gaspirin, Gattex, GBR500, GBR500 antibody, GBT009, G-CSF, GED0301, GED0414, Gefenec, Gelofen, Genepril, Gengraf, Genimune, Geniquin, Genotropin, Genz29155, Gerbin, Gerbin, gevokizumab, GF01564600, Gilenia, Gilenya, givinostat, GL0050, GL2045, glatiramer acetate, Globulin, Glortho Forte, Glovalox, Glovenin-I, GLPG0259, GLPG0555, GLPG0634, GLPG0778, GLPG0974, Gluco, Glucocerin, glucosamine, glucosamine hydrochloride, glucosamine sulfate, Glucotin, Gludex, Glutilage, GLY079, GLY145, Glycanic, Glycefort up, Glygesic, Glysopep, GMCSF Antibody, GMI1010, GMI1011, GMI1043, GMR321, GN4001, Goanna Salve, Goflex, gold sodium thiomalate, golimumab, GP2013, GPCR modulator, GPR15 Antagonist, GPR183 antagonist, GPR32 antagonist, GPR83 antagonist, G-protein Coupled Receptor Antagonists, Graceptor, Graftac, granulocyte colony-stimulating factor antibody, granulocyte-macrophage colony-stimulating factor antibody, Gravx, GRC4039, Grelyse, GS101, GS9973, GSC100, GSK1605786, GSK1827771, GSK2136525, GSK2941266, GSK315234, GSK681323, GT146, GT442, Gucixiaotong, Gufisera, Gupisone, gusperimus hydrochloride, GW274150, GW3333, GW406381, GW856553, GWB78, GXP04, Gynestrel, Haloart, halopredone acetate, Haloxin, HANALL, Hanall Soludacortin, Havisco, Hawon Bucillamin, HB802, HC31496, HCQ 200, HD104, HD203, HD205, HDAC inhibitor, HE2500, HE3177, HE3413, Hecoria, Hectomitacin, Hefasolon, Helen, Helenil, HemaMax, Hematom, hematopoietic stem cells, Hematrol, Hemner, Hemril, heparinoid, Heptax, HER2 Antibody, Herponil, hESC Derived Dendritic Cells, hESC Derived Hematopoietic stem cells, Hespercorbin, Hexacorton, Hexadrol, hexetidine, Hexoderm, Hexoderm Salic, HF0220, HF1020, HFT-401, hG-CSFR ED Fc, Hiberna, high mobility group box 1 antibody, Hiloneed, Hinocam, hirudin, Hirudoid, Hison, Histamine H4 Receptor Antagonist, Hitenercept, Hizentra, HL036, HL161, HMPL001, HMPL004, HMPL004, HMPL011, HMPL342, HMPL692, honey bee venom, Hongqiang, Hotemin, HPH116, HTI101, HuCAL Antibody, Human adipose mesenchymal stem cells, anti-MHC class II monoclonal antibody, Human Immunoglobulin, Human Placenta Tissue Hydrolysate, HuMaxCD4, HuMax-TAC, Humetone, Humicade, Humira, Huons Betamethasone sodium phosphate, Huons dexamethasone sodium phosphate, Huons Piroxicam, Huons Talniflumate, Hurofen, Huruma, Huvap, HuZAF, HX02, Hyalogel, hyaluronate sodium, hyaluronic acid, hyaluronidase, Hyaron, Hycocin, Hycort, Hy-Cortisone, hydrocortisone, hydrocortisone acetate, hydrocortisone butyrate, hydrocortisone hemisuccinate, hydrocortisone sodium phosphate, hydrocortisone sodium succinate, Hydrocortistab, Hydrocortone, Hydrolin, Hydroquine, Hydro-Rx, Hydrosone HIKMA, hydroxychloroquine, hydroxychloroquine sulfate, Hylase Dessau, HyMEX, Hypen, HyQ, Hysonate, HZN602, I.M.75, IAP Inhibitors, Ibalgin, Ibalgin, Ibex, ibrutinib, IBsolvMIR, Ibu, Ibucon, Ibudolor, Ibufen, Ibuflam, Ibuflex, Ibugesic, Ibu-Hepa, Ibukim, Ibumal, Ibunal, Ibupental, Ibupril, Ibuprof, ibuprofen, Ibuscent, Ibusoft, Ibusuki Penjeong, Ibususpen, Ibutard, Ibutop, Ibutop, Ibutrex, IC487892, ichthammol, ICRAC Blocker, IDEC131, IDECCE9.1, Ides, Idicin, Idizone, IDN6556, Idomethine, IDR1, Idyl SR, Ifen, iguratimod, IK6002, IKK-beta inhibitor, IL17 Antagonist, IL-17 Inhibitor, IL-17RC, IL18, IL1Hy1, ILIR1, IL-23 Adnectin, IL23 Inhibitor, IL23 Receptor Antagonist, IL-31 mAb, IL-6 Inhibitor, IL6Qb, Ilacox, Ilaris, ilodecakin, ILV094, ILV095, Imaxetil, IMD0560, IMD2560, Imesel Plus, Iminoral, Immodin, IMMU103, IMMU106, Immucept, Immufine, Immunex Syrup, immunoglobulin, immunoglobulin G, Immunoprin, ImmunoRel, Immurin, IMO8400, IMP731 antibody, Implanta, Imunocell, Imuran, Imurek, Imusafe, Imusporin, Imutrex, IN0701, Inal, INCB039110, INCB18424, INCB28050, INCB3284, INCB3344, Indexon, Indic, Indo, Indo-A, Indobid, Indo-Bros, Indocaf, Indocarsil, Indocid, Indocin, Indomehotpas, Indomen, Indomet, Indometacin, indomethacin, Indomethasone, Indometin, Indomin, Indopal, Indoron, Indotroxin, INDUS830, INDUS83030, Infladase, Inflamac, Inflammasome inhibitor, Inflavis, Inflaxen, Inflectra, infliximab, Ingalipt, Inicox dp, Inmecin, Inmunoartro, Innamit, InnoD06006, INO7997, Inocin, Inoten, Inovan, Inpra, Inside Pap, Insider-P, Instacyl, Instracool, Intafenac, Intaflam, Inteban, Inteban Spansule, integrin, alpha 1 antibody, integrin, alpha 2 antibody, Intenurse, interferon alfa, interferon beta-la, interferon gamma, interferon gamma antibody, Interking, interleukin 1 Hy1, interleukin 1 antibody, interleukin 1 receptor antibody, interleukin 1, beta antibody, interleukin 10, interleukin 10 antibody, interleukin 12, interleukin 12 antibody, interleukin 13 antibody, interleukin 15 antibody, interleukin 17 antibody, interleukin 17 receptor C, interleukin 18, interleukin 18 binding protein, interleukin 18 antibody, interleukin 2 receptor, alpha antibody, interleukin 20 antibody, Interleukin 21 mAb, interleukin 23 aptamer, interleukin 31 antibody, interleukin 34, Interleukin 6 Inhibitor, interleukin 6 antibody, interleukin 6 receptor antibody, interleukin 7, interleukin 7 receptor antibody, interleukin 8, interleukin 8 antibody, interleukin-18 antibody, Intidrol, Intradex, Intragam P, Intragesic, Intraglobin F, Intratect, Inzel, Iomab B, IOR-T3, IP751, IPH2201, IPH2301, IPH24, IPH33, IPI145, Ipocort, IU.S. Plant Pat. No. 201,007, I-Profen, Iprox, Ipson, Iputon, IRAK4 Inhibitor, Iremod, Irtonpyson, IRX3, IRX5183, ISA247, ISIS104838, ISIS2302, ISISCRPRx, Ismafron, IsoQC inhibitor, Isox, ITF2357, Iveegam EN, Ivepred, IVIG-SN, IW001, Izilox, J607Y, J775Y, JAK Inhibitor, JAK3 inhibitor, JAK3 kinase inhibitor, JI3292, JI4135, Jinan Lida, JNJ10329670, JNJ18003414, JNJ26528398, JNJ27390467, JNJ28838017, JNJ31001958, JNJ38518168, JNJ39758979, JNJ40346527, JNJ7777120, JNT-Plus, Joflam, Joint Glucosamin, Jointec, Jointstem, Joinup, JPE1375, JSM10292, JSM7717, JSM8757, JTE051, JTE052, JTE522, JTE607, Jusgo, K412, K832, Kaflam, KAHR101, KAHR102, KAI9803, Kalymin, Kam Predsol, Kameton, KANAb071, Kappaproct, KAR2581, KAR3000, KAR3166, KAR4000, KAR4139, KAR4141, KB002, KB003, KD7332, KE298, keliximab, Kemanat, Kemrox, Kenacort, Kenalog, Kenaxir, Kenketsu Venoglobulin-IH, Keplat, Ketalgipan, Keto Pine, Keto, Ketobos, Ketofan, Ketofen, Ketolgan, Ketonal, Ketoplus Kata Plasma, ketoprofen, Ketores, Ketorin, ketorolac, ketorolac tromethamine, Ketoselect, Ketotop, Ketovail, Ketricin, Ketroc, Ketum, Keyi, Keyven, KF24345, K-Fenac, K-Fenak, K-Gesic, Kifadene, Kilcort, Kildrol, KIM127, Kimotab, Kinase Inhibitor 4SC, Kinase N, Kincort, Kindorase, Kineret, Kineto, Kitadol, Kitex, Kitolac, KLK1 Inhibitor, Klofen-L, Klotaren, KLS-40or, KLS-40ra, KM277, Knavon, Kodolo orabase, Kohakusanin, Koide, Koidexa, Kolbet, Konac, Kondro, Kondromin, Konshien, Kontab, Kordexa, Kosa, Kotase, KPE06001, KRP107, KRP203, KRX211, KRX252, KSB302, K-Sep, Kv 1.3 Blocker, Kv1.3 4SC, Kv1.3 inhibitor, KVK702, Kynol, L156602, Labizone, Labohydro, Labopen, Lacoxa, Lamin, Lamit, Lanfetil, laquinimod, larazotide acetate, LAS186323, LAS187247, LAS41002, Laticort, LBEC0101, LCP3301, LCP-Siro, LCP-Tacro, LCsA, LDP392, Leap-S, Ledercort, Lederfen, Lederlon, Lederspan, Lefenine, leflunomide, Leflux, Lefno, Lefra, Leftose, Lefumide, Lefunodin, Lefva, lenalidomide, lenercept, LentiRA, LEO15520, Leodase, Leukine, Leukocyte function-associated antigen-1 antagonist, leukocyte immunoglobulin-like receptor, subfamily A, member 4 antibody, Leukothera, leuprolide acetate, levalbuterol, levomenthol, LFA-1 Antagonist, LFA451, LFA703, LFA878, LG106, LG267 Inhibitors, LG688 Inhibitors, LGD5552, Li Life, LidaMantle, Lidex, lidocaine, lidocaine hydrochloride, Lignocaine hydrochloride, LIM0723, LIM5310, Limethason, Limus, Limustin, Lindac, Linfonex, Linola acute, Lipcy, lisofylline, Listran, Liver X Receptor modulator, Lizak, LJP1207, LJP920, Lobafen, Lobu, Locafluo, Localyn, Locaseptil-Neo, Locpren, Lodine, Lodotra, Lofedic, Loflam, Lofnac, Lolcam, Lonac, lonazolac calcium, Loprofen, Loracort, Lorcam, Lorfenamin, Lorinden Lotio, Lorncrat, lornoxicam, Lorox, losmapimod, loteprednol etabonate, Loteprednol, Lotirac, Low Molecular Ganoderma lucidum Polysaccharide, Loxafen, Loxfenine, Loxicam, Loxofen, Loxonal, Loxonin, loxoprofen sodium, Loxoron, LP183A1, LP183A2, LP204A1, LPCN1019, LT1942, LT1964, LTNS101, LTNS103, LTNS106, LTNS108, LTS1115, LTZMP001, Lubor, lumiracoxib, Lumitect, LX2311, LX2931, LX2932, LY2127399, LY2189102, LY2439821, LY294002, LY3009104, LY309887, LY333013, lymphocyte activation gene 3 antibody, Lymphoglobuline, Lyser, lysine aspirin, Lysobact, Lysoflam, Lysozyme hydrochloride, M3000, M834, M923, mAb hG-CSF, MABP1, macrophage migration inhibitory factor antibody, Maitongna, Majamil prolongatum, major histocompatibility complex class II DR antibody, major histocompatibility complex class II antibody, Malidens, Malival, mannan-binding lectin, mannan-binding lectin-associated serine protease-2 antibody, MapKap Kinase 2 Inhibitor, maraviroc, Marlex, masitinib, Maso, MASP2 antibody, MAT304, Matrix Metalloprotease Inhibitor, mavrilimumab, Maxiflam, Maxilase, Maximus, Maxisona, Maxius, Maxpro, Maxrel, Maxsulid, Maxyl2, Maxy30, MAXY4, Maxy735, Maxy740, Mayfenamic, MB11040, MBPY003b, MCAF5352A, McCam, McRofy, MCS18, MD707, MDAM, MDcort, MDR06155, MDT012, Mebicam, Mebuton, meclofenamate sodium, Meclophen, Mecox, Medacomb, Medafen, Medamol, Medesone, MEDI2070, MEDI5117, MEDI541, MEDI552, MEDI571, Medicox, Medifen, Medisolu, Medixon, Mednisol, Medrol, Medrolon, medroxyprogesterone acetate, Mefalgin, mefenamic acid, Mefenix, Mefentan, Meflen, Mefnetra forte, Meftagesic-DT, Meftal, Megakaryocyte Growth and Development Factor, Megaspas, Megaster, megestrol acetate, Meite, Meksun, Melbrex, Melcam, Melcam, Melflam, Melic, Melica, Melix, Melocam, Melocox, Mel-One, Meloprol, Melosteral, Melox, Meloxan, Meloxcam, Meloxic, Meloxicam, Meloxifen, Meloxin, Meloxiv, Melpred, Melpros, Melurjin, Menamin, Menisone, Menthomketo, Menthoneurin, Mentocin, Mepa, Mepharen, meprednisone, Mepresso, Mepsolone, mercaptopurine, Mervan, Mesadoron, mesalamine, Mesasal, Mesatec, Mesenchymal Precursor Cells, mesenchymal stem cell, Mesipol, Mesren, Mesulan, Mesulid, Metacin, Metadaxan, Metaflex, Metalcaptase, metalloenzyme inhibitors, Metapred, Metax, Metaz, Meted, Metedic, Methacin, Methaderm, Methasone, Methotrax, methotrexate, methotrexate sodium, Methpred, Methyl prednisolone acetate, methyl salicylate, methyl sulphonyl methane, Methylon, Methylpred, methylprednisolone, methylprednisolone acetate, methylprednisolone sodium succinate, methylprednisolone succinate, Methylprednisolone, Methysol, Metindol, Metoart, Metoject, Metolate, Metoral, Metosyn, Metotab, Metracin, Metrex, metronidazole, Metypred, Mevamox, Mevedal, Mevilox, Mevin SR, Mexilal, Mexpharm, Mext, Mextran, MF280, M-FasL, MHC class II beta chain peptide, Micar, Miclofen, Miclofenac, Micofenolato Mofetil, Micosone, Microdase, microRNA 181a-2 oligonucleotide, MIF Inhibitors, MIFQb, MIKA-Ketoprofen, Mikametan, milodistim, Miltax, Minafen, Minalfen, Minalfene, Minesulin, Minocort, Mioflex, Miolox, Miprofen, Miridacin, Mirloks, Misoclo, Misofenac, MISTB03, MISTB04, Mitilor, mizoribine, MK409, MK0359, MK0812, MK0873, MK2 Inhibitors, MK50, MK8457, MK8808, MKC204, MLN0002, MLN0415, MLN1202, MLN273, MLN3126, MLN3701, MLN3897, MLNM002, MM093, MM7XX, MN8001, Mobic, Mobicam, Mobicox, Mobifen Plus, Mobilat, Mobitil, Mocox, Modigraf, Modrasone, Modulin, Mofecept, Mofetyl, mofezolac sodium, Mofilet, Molace, molgramostim, Molslide, Momekin, Momen Gele, Moment 100, Momesone, Momesun, Mometamed, mometasone, mometasone furoate, Monimate, monosodium alpha-luminol, Mopik, MOR103, MOR104, MOR105, MOR208 antibody, MORAb022, Moricam, morniflumate, Mosuolit, Motoral, Movaxin, Mover, Movex, Movix, Movoxicam, Mox Forte, Moxen, moxifloxacin hydrochloride, Mozobil, MP, MP0210, MP0270, MP1000, MP1031, MP196, MP435, MPA, mPGES-1 inhibitor, MPSS, MRX7EAT, MSL, MT203, MT204, mTOR Inhibitor, MTRX1011A, Mucolase, Multicort, MultiStem, muramidase, muramidase, muramidase hydrochloride, muromonab-CD3, Muslax, Muspinil, Mutaze, Muvera, MX68, Mycept, Mycocell, Mycocept, Mycofenolatmofetil Actavis, Mycofet, Mycofit, Mycolate, Mycoldosa, Mycomun, Myconol, mycophenolate mofetil, mycophenolate sodium, mycophenolic acid, Mycotil, myeloid progenitor cells, Myfenax, Myfetil, Myfortic, Mygraft, Myochrysine, Myocrisin, Myprodol, Mysone, nab-Cyclosporine, Nabentac, nabiximols, Nabton, Nabuco, Nabucox, Nabuflam, Nabumet, nabumetone, Nabuton, Nac Plus, Nacta, Nacton, Nadium, Naklofen SR, NAL1207, NAL1216, NAL1219, NAL1268, NAL8202, Nalfon, Nalgesin S, namilumab, Namsafe, nandrolone, Nanocort, Nanogam, Nanosomal Tacrolimus, Napageln, Napilac, Naprelan, Napro, Naprodil, Napronax, Napropal, Naproson, Naprosyn, Naproval, Naprox, naproxen, naproxen sodium, Naproxin, Naprozen, Narbon, Narexsin, Naril, Nasida, natalizumab, Naxdom, Naxen, Naxin, Nazovel, NC2300, ND07, NDC01352, Nebumetone, NecLipGCSF, Necsulide, Necsunim, Nelsid-S, Neo Clobenate, Neo Swiflox FC, Neocoflan, Neo-Drol, Neo-Eblimon, Neo-Hydro, Neoplanta, Neoporine, Neopreol, Neoprox, Neoral, Neotrexate, Neozen, Nepra, Nestacort, Neumega, Neupogen, Neuprex, Neurofenac, Neurogesic, Neurolab, Neuroteradol, Neuroxicam, Neutalin, neutrazumab, Neuzym, New Panazox, Newfenstop, NewGam, Newmafen, Newmatal, Newsicam, NEX1285, sFcRIIB, Nextomab, NF-kappaB Inhibitor, NF-kB inhibitor, NGD20001, NHP554B, NHP554P, NI0101 antibody, NI0401, NI0501 antibody, NI0701, NI071, NI1201 antibody, NI1401, Nicip, Niconas, Nicool, NiCord, Nicox, Niflumate, Nigaz, Nikam, Nilitis, Nimace, Nimaid, Nimark-P, Nimaz, Nimcet Juicy, Nime, Nimed, Nimepast, nimesulide, Nimesulix, Nimesulon, Nimica Plus, Nimkul, Nimlin, Nimnat, Nimodol, Nimpidase, Nimsaid-S, Nimser, Nimsy-SP, Nimupep, Nimusol, Nimutal, Nimuwin, Nimvon-S, Nincort, Niofen, Nipan, Nipent, Nise, Nisolone, Nisopred, Nisoprex, Nisulid, nitazoxanide, Nitcon, nitric oxide, Nizhvisal B, Nizon, NL, NMR1947, NN8209, NN8210, NN8226, NN8555, NN8765, NN8828, NNC014100000100, NNC051869, Noak, Nodevex, Nodia, Nofenac, Noflagma, Noflam, Noflamen, Noflux, Non-antibacterial Tetracyclines, Nonpiron, Nopain, Normferon, Notpel, Notritis, Novacort, Novagent, Novarin, Novigesic, NOXA12, NOXD19, Noxen, Noxon, NPI1302a-3, NPI1342, NPI1387, NPI1390, NPRCS1, NPRCS2, NPRCS3, NPRCS4, NPRCS5, NPRCS6, NPS3, NPS4, nPT-ery, NU3450, nuclear factor NF-kappa-B p65 subunit oligonucleotide, Nucort, Nulojix, Numed-Plus, Nurokind Ortho, Nusone-H, Nutrikemia, Nuvion, NV07alpha, NX001, Nyclobate, Nyox, Nysa, Obarcort, OC002417, OC2286, ocaratuzumab, OCTSG815, Oedemase, Oedemase-D, ofatumumab, Ofgyl-O, Ofvista, OHR118, OKi, Okifen, Oksamen, Olai, olokizumab, Omeprose E, Omnacortil, Omneed, Omniclor, Omnigel, Omniwel, onercept, ONO057, ONS1210, ONS1220, Ontac Plus, Ontak, ONX0914, OPC6535, opebacan, OPN101, OPN201, OPN302, OPN305, OPN401, oprelvekin, OPT66, Optifer, Optiflur, OptiMIRA, Orabase Hca, Oradexon, Oraflex, OralFenac, Oralog, Oralpred, Ora-sed, Orasone, orBec, Orbone forte, Orcl, ORE10002, ORE10002, Orencia, Org214007, Org217993, Org219517, Org223119, Org37663, Org39141, Org48762, Org48775, Orgadrone, Ormoxen, Orofen Plus, Oromylase Biogaran, Orthal Forte, Ortho Flex, Orthoclone OKT3, Orthofen, Orthoflam, Orthogesic, Orthoglu, Ortho-II, Orthomac, Ortho-Plus, Ortinims, Ortofen, Orudis, Oruvail, OS2, Oscart, Osmetone, Ospain, Ossilife, Ostelox, Osteluc, Osteocerin, osteopontin, Osteral, otelixizumab, Otipax, Ou Ning, OvaSave, OX40 Ligand Antibody, Oxa, Oxagesic CB, Oxalgin DP, oxaprozin, OXCQ, Oxeno, Oxib MD, Oxibut, Oxicam, Oxiklorin, Oximal, Oxynal, oxyphenbutazone, Oxyphenbutazone, ozoralizumab, P13 peptide, P1639, P21, P2X7 Antagonists, p38 Alpha Inhibitor, p38 Antagonist, p38 MAP kinase inhibitor, p38alpha MAP Kinase Inhibitor, P7 peptide, P7170, P979, PA401, PA517, Pabi-dexamethasone, PAC, PAC10649, paclitaxel, Painoxam, Paldon, Palima, pamapimod, Pamatase, Panafcort, Panafcortelone, Panewin, PanGraf, Panimun Bioral, Panmesone, Panodin SR, Panslay, Panzem, Panzem NCD, PAP1, papain, Papirzin, Pappen K Pap, Paptinim-D, paquinimod, PAR2 Antagonist, Paracetamol, Paradic, Parafen TAJ, Paramidin, Paranac, Parapar, Parci, parecoxib, Parixam, Parry-S, Partaject Busulfan, pateclizumab, Paxceed, PBI0032, PBI1101, PBI1308, PBI1393, PBI1607, PBI1737, PBI2856, PBI4419, PBI4419, P-Cam, PCI31523, PCI32765, PCI34051, PCI45261, PCI45292, PCI45308, PD360324, PD360324, PDA001, PDE4 inhibitor, PDE-IV Inhibitor, PDL241 antibody, PDL252, Pediapred, Pefree, pegacaristim, Peganix, Peg-Interleukin 12, pegsunercept, Pegsunercept, PEGylated arginine deiminase, peldesine, pelubiprofen, Penacle, penicillamine, Penostop, Pentalgin, Pentasa, Pentaud, pentostatin, Peon, Pepdase, Pepser, Peptirase, Pepzen, Pepzol, Percutalgine, Periochip, Peroxisome Proliferator Activated Receptor gamma modulators, Petizene, PF00344600, PF04171327, PF04236921, PF04308515, PF05230905, PF05280586, PF251802, PF3475952, PF3491390, PF3644022, PF4629991, PF4856880, PF5212367, PF5230896, PF547659, PF755616, PF9184, PG27, PG562, PG760564, PG8395, PGE3935199, PGE527667, PH5, PH797804, PHA408, Pharmaniaga Mefenamic acid, Pharmaniaga Meloxicam, Pheldin, Phenocept, phenylbutazone, PHY702, PI3K delta inhibitor, PI3K Gamma/Delta Inhibitor, PI3K Inhibitor, Picalm, pidotimod, piketoprofen, Pilelife, Pilopil, Pilovate, pimecrolimus, Pipethanen, Piractam, Pirexyl, Pirobet, Piroc, Pirocam, Pirofel, Pirogel, Piromed, Pirosol, Pirox, Piroxen, Piroxicam, piroxicam betadex, Piroxifar, Piroxil, Piroxim, Pixim, Pixykine, PKC Theta Inhibitor, PL3100, PL5100 Diclofenac, Placenta Polypeptide, Plaquenil, plerixafor, Plocfen, PLR14, PLR18, Plutin, PLX3397, PLX5622, PLX647, PLX-BMT, pms-Diclofenac, pms-Ibuprofen, pms-Leflunomide, pms-Meloxicam, pms-Piroxicam, pms-Prednisolone, pms-Sulfasalazine, pms-Tiaprofenic, PMX53, PN0615, PN100, PN951, podofilox, POL6326, Polcortolon, Polyderm, Polygam S/D, Polyphlogin, Poncif, Ponstan, Ponstil Forte, Porine-A Neoral, Potaba, potassium aminobenzoate, Potencort, Povidone, povidone iodine, pralnacasan, Prandin, Prebel, Precodil, Precortisyl Forte, Precortyl, Predfoam, Predicort, Predicorten, Predilab, Predilone, Predmetil, Predmix, Predna, Prednesol, Predni, prednicarbate, Prednicort, Prednidib, Prednifarma, Prednilasca, prednisolone, Deltacortril (prednisolone), prednisolone acetate, prednisolone sodium phosphate, prednisolone sodium succinate, prednisolone sodium succinate, prednisone, prednisone acetate, Prednitop, Prednol-L, Prednox, Predone, Predonema, Predsol, Predsolone, Predsone, Predval, Preflam, Prelon, Prenaxol, Prenolone, Preservex, Preservin, Presol, Preson, Prexige, Priliximab, Primacort, Primmuno, Primofenac, prinaberel, Privigen, Prixam, Probuxil, Procarne, Prochymal, Procider-EF, Proctocir, Prodase, Prodel B, Prodent, Prodent Verde, Proepa, Profecom, Profenac L, Profenid, Profenol, Proflam, Proflex, Progesic Z, proglumetacin, proglumetacin maleate, Prograf, Prolase, Prolixan, promethazine hydrochloride, Promostem, Promune, PronaB, pronase, Pronat, Prongs, Pronison, Prontoflam, Propaderm-L, Propodezas, Propolisol, Proponol, propyl nicotinate, Prostaloc, Prostapol, Protacin, Protase, Protease Inhibitors, Protectan, Proteinase Activated Receptor 2 Inhibitor, Protofen, Protrin, Proxalyoc, Proxidol, Proxigel, Proxil, Proxym, Prozym, PRT062070, PRT2607, PRTX100, PRTX200, PRX106, PRX167700, Prysolone, PS031291, PS375179, PS386113, PS540446, PS608504, PS826957, PS873266, Psorid, PT, PT17, PTL101, P-Transfer Factor peptides, PTX3, Pulminiq, Pulsonid, Purazen, Pursin, PVS40200, PX101, PX106491, PX114, PXS2000, PXS2076, PYM60001, Pyralvex, Pyranim, pyrazinobutazone, Pyrenol, Pyricam, Pyrodex, Pyroxi-Kid, QAX576, Qianbobiyan, QPI1002, QR440, qT3, Quiacort, Quidofil, R107s, R125224, R1295, R132811, R1487, R1503, R1524, R1628, R333, R348, R548, R7277, R788, rabeximod, Radix Isatidis, Radofen, Raipeck, Rambazole, Randazima, Rapacan, Rapamune, Raptiva, Ravax, Rayos, RDEA119, RDEA436, RDP58, Reactine, Rebif, REC200, Recartix-DN, receptor for advanced glycation end products antibody, Reclast, Reclofen, recombinant HSA-TIMP-2, recombinant human alkaline Phosphatase, recombinant Interferon Gamma, Recominant human alkaline phosphatase, Reconil, Rectagel HC, Recticin, Recto Menaderm, Rectos, Redipred, Redolet, Refastin, Regenica, REGN88, Relafen, Relaxib, Relev, Relex, Relifen, Relifex, Relitch, Rematof, remestemcel-1, Remesulidum, Remicade® (infliximab), Remsima, Remsima, Remsima, ReN1869, Renacept, Renfor, Renodapt, Renodapt-S, Renta, Reosan, Repare-AR, Reparilexin, reparixin, Repertaxin, Repisprin, Resochin, Resol, resolvin El, Resurgil, Re-tin-colloid, Retoz, Reumacap, Reumacon, Reumadolor, Reumador, Reumanisal, Reumazin, Reumel, Reumotec, Reuquinol, revamilast, Revascor, Reviroc, Revlimid, Revmoksikam, Rewalk, Rexalgan, RG2077, RG3421, RG4934 antibody, RG7416, RG7624, Rheila, Rheoma, Rheprox, Rheudenolone, Rheufen, Rheugesic, Rheumacid, Rheumacort, Rheumatrex, Rheumesser, Rheumid, Rheumon, Rheumox, Rheuoxib, Rhewlin, Rhucin, RhuDex, Rhulef, Ribox, Ribunal, Ridaura, rifaximin, rilonacept, rimacalib, Rimase, Rimate, Rimatil, Rimesid, risedronate sodium, Ritamine, Rito, Rituxan, rituximab, RNS60, RO1138452, Ro313948, RO3244794, RO5310074, Rob803, Rocamix, Rocas, Rofeb, rofecoxib, Rofee, Rofewal, Roficip Plus, Rojepen, Rokam, Rolodiquim, Romacox Fort, Romatim, romazarit, Ronaben, ronacaleret, Ronoxcin, ROR Gamma T Antagonist, ROR gamma t inverse agonists, Rosecin, rosiglitazone, Rosmarinic acid, Rotan, Rotec, Rothacin, Roxam, Roxib, Roxicam, Roxopro, Roxygin DT, RP54745, RPI78, RPI78M, RPI78MN, RPIMN, RQ00000007, RQ00000008, RTA402, R-Tyflam, Rubicalm, Rubifen, Ruma pap, Rumalef, Rumidol, Rumifen, Runomex, rusalatide acetate, ruxolitinib, RWJ445380, RX10001, Rycloser MR, Rydol, SIP Receptor Agonists, SIP Receptor Modulators, SIP1 Agonist, SIP1 receptor agonist, S2474, S3013, SA237, SA6541, Saaz, S-adenosyl-L-methionine-sulfate-p-toluene sulfonate, Sala, Salazidin, Salazine, Salazopyrin, Salcon, Salicam, salsalate, Sameron, SAN300, Sanaven, Sandimmun, Sandoglobulin, Sanexon, SangCya, SAR153191, SAR302503, SAR479746, Sarapep, sargramostim, Sativex, Savantac, Save, Saxizon, Sazo, SB1578, SB210396, SB217969, SB242235, SB273005, SB281832, SB683698, SB751689, SBI087, SC080036, SC12267, SC409, Scaflam, SCD ketoprofen, SCIO323, SCIO469, SD-15, SD281, SDP051 antibody, Sd-IxRNA, secukinumab, Sedase, Sedilax, Sefdene, Seizyme, SEL113, Seladin, Selecox, selectin P ligand antibody, Glucocorticoid Receptor Agonist, Selectofen, Selektine, SelK1 antibody, Seloxx, Selspot, Selzen, Selzenta, Selzentry, semapimod, semapimod hydrochloride, semparatide, Semparatide, Senafen, Sendipen, Senterlic, SEP119249, Sepdase, Septirose, Seractil, Serafen-P, Serase, Seratid D, Seratiopeptidase, Serato-M, Seratoma Forte, Serazyme, Serezon, Sero, Serodase, Serpicam, Serra, serrapeptase, Serratin, Serratiopeptidase, Serrazyme, Servisone, Seven E P, SGI1252, SGN30, SGN70, SGX203, shark cartilage extract, Sheril, Shield, Shifazen, Shifazen-Fort, Shincort, Shincort, Shiosol, ShK186, Shuanghuangxiaoyan, SI615, SI636, Sigmasporin, Sigmasporin, SIM916, Simpone, Simulect, Sinacort, Sinalgia, Sinapol, Sinatrol, Sinsia, siponimod, Sirolim, sirolimus, Siropan, Sirota, Sirova, sirukumab, Sistal Forte, SKF105685, SKF105809, SKF106615, SKF86002, Skinalar, Skynim, Skytrip, SLAM family member 7 antibody, Slo-indo, SM101, SM201 antibody, SM401, SMAD family member 7 oligonucleotide, SMART Anti-IL-12 Antibody, SMP114, SNO030908, SNO070131, sodium aurothiomalate, sodium chondroitin sulfate, sodium deoxyribonucleotide, sodium gualenate, sodium naproxen, sodium salicylate, Sodixen, Sofeo, Soleton, Solhidrol, Solicam, Soliky, Soliris, Sol-Melcort, Solomet, Solondo, Solone, Solu-Cort, Solu-Cortef, Solu-Decortin H, Solufen, Solu-Ket, Solumark, Solu-Medrol, Solupred, Somalgen, somatropin, Sonap, Sone, sonepcizumab, Sonexa, Sonim, Sonim P, Soonil, Soral, Sorenil, sotrastaurin acetate, SP-10, SP600125, Spanidin, SP-Cortil, SPD550, Spedace, sperm adhesion molecule 1, Spictol, spleen tyrosine kinase oligonucleotide, Sporin, S-prin, SPWF1501, SQ641, SQ922, SR318B, SR9025, SRT2104, SSR150106, SSR180575, SSS07 antibody, ST1959, STA5326, stabilin 1 antibody, Stacort, Stalogesic, stanozolol, Staren, Starmelox, Stedex IND-SWIFT, Stelara, Stemin, Stenirol, Sterapred, Steriderm S, Sterio, Sterisone, Steron, stichodactyla helianthus peptide, Stickzenol A, Stiefcortil, Stimulan, STNM01, Store Operated Calcium Channel (SOCC) Modulator, STP432, STP900, Stratasin, Stridimmune, Strigraf, SU Medrol, Subreum, Subuton, Succicort, Succimed, Sulan, Sulcolon, Sulfasalazin Heyl, Sulfasalazin, sulfasalazine, Sulfovit, Sulidac, Sulide, sulindac, Sulindex, Sulinton, Sulphafine, Sumilu, SUN597, Suprafen, Supretic, Supsidine, Surgam, Surgamine, Surugamu, Suspen, Suton, Suvenyl, Suwei, SW Dexasone, Syk Family Kinase Inhibitor, Syn1002, Synacran, Synacthen, Synalar C, Synalar, Synavive, Synercort, Sypresta, T cell cytokine-inducing surface molecule antibody, T cell receptor antibody, T5224, T5226, TA101, TA112, TA383, TA5493, tabalumab, Tacedin, Tacgraf, TACIFc5, Tacrobell, Tacrograf, Tacrol, tacrolimus, Tadekinig alpha, Tadolak, TAFA93, Tafirol Artro, Taizen, TAK603, TAK715, TAK783, Takfa, Taksta, talarozole, Talfin, Talmain, talmapimod, Talmea, Talnif, talniflumate, Talos, Talpain, Talumat, Tamalgen, Tamceton, Tamezon, Tandrilax, tannins, Tannosynt, Tantum, tanzisertib, Tapain-beta, Tapoein, Tarenac, tarenflurbil, Tarimus, Tarproxen, Tauxib, Tazomust, TBR652, TC5619, T-cell, immune regulator 1, ATPase, H+ transporting, lysosomal V0 subunit A3 antibody, TCK1, T-cort, T-Dexa, Tecelac, Tecon, teduglutide, Teecort, Tegeline, Tementil, temoporfin, Tencam, Tendrone, Tenefuse, Tenfly, tenidap sodium, Tenocam, Tenoflex, Tenoksan, Tenotil, tenoxicam, Tenoxim, Tepadina, Teracort, Teradol, tetomilast, TG0054, TG1060, TG20, TG20, tgAAC94, Th1/Th2 Cytokine Synthase Inhibitor, Th-17 cell inhibitors, Thalido, thalidomide, Thalomid, Themisera, Thenil, Therafectin, Therapyace, thiarabine, Thiazolopyrimidines, thioctic acid, thiotepa, THR090717, THR0921, Threenofen, Thrombate III, Thymic peptide, Thymodepressin, Thymogam, Thymoglobulin, Thymoglobuline, Thymoject thymic peptides, thymomodulin, thymopentin, thymopolypetides, tiaprofenic acid, tibezonium iodide, Ticoflex, tilmacoxib, Tilur, T-immune, Timocon, Tiorase, Tissop, TKB662, TL011, TLR4 antagonists, TLR8 inhibitor, TM120, TM400, TMX302, TNF Alpha inhibitor, TNF alpha-TNF receptor antagonist, TNF antibody, TNF receptor superfamily antagonists, TNF TWEAK Bi-Specific, TNF-Kinoid, TNFQb, TNFR1 antagonist, TNR001, TNX100, TNX224, TNX336, TNX558, tocilizumab, tofacitinib, Tokuhon happ, TOL101, TOL102, Tolectin, ToleriMab, Tolerostem, Tolindol, toll-like receptor 4 antibody, toll-like receptor antibody, tolmetin sodium, Tongkeeper, Tonmex, Topflame, Topicort, Topleucon, Topnac, Toppin Ichthammol, toralizumab, Toraren, Torcoxia, Toroxx, Tory, Toselac, Totaryl, Touch-med, Touchron, Tovok, Toxic apis, Toyolyzom, TP4179, TPCA1, TPI526, TR14035, Tradil Fort, Traficet-EN, Tramace, tramadol hydrochloride, tranilast, Transimune, Transporina, Tratul, Trexall, Triacort, Triakort, Trialon, Triam, triamcinolone, triamcinolone acetate, triamcinolone acetonide, triamcinolone acetonide acetate, triamcinolone hexacetonide, Triamcort, Triamsicort, Trianex, Tricin, Tricort, Tricortone, TricOs T, Triderm, Trilac, Trilisate, Trinocort, Trinolone, Triolex, triptolide, Trisfen, Trivaris, TRK170, TRK530, Trocade, trolamine salicylate, Trolovol, Trosera, Trosera D, Troycort, TRX1 antibody, TRX4, Trymoto, Trymoto-A, TT301, TT302, TT32, TT32, TT33, TTI314, tumor necrosis factor, tumor necrosis factor 2-methoxyethyl phosphorothioate oligonucleotide, tumor necrosis factor antibody, tumor necrosis factor kinoid, tumor necrosis factor oligonucleotide, tumor necrosis factor receptor superfamily, member 1B antibody, tumor necrosis factor receptor superfamily 1B oligonucleotide, tumor necrosis factor superfamily, member 12 antibody, tumor necrosis factor superfamily, member 4 antibody, tumor protein p53 oligonucleotide, tumour necrosis factor alpha antibody, TuNEX, TXA127, TX-RAD, TYK2 inhibitors, Tysabri, ubidecarenone, Ucerase, ulodesine, Ultiflam, Ultrafastin, Ultrafen, Ultralan, U-Nice-B, Uniplus, Unitrexate, Unizen, Uphaxicam, UR13870, UR5269, UR67767, Uremol-HC, Urigon, U-Ritis, ustekinumab, V85546, Valcib, Valcox, valdecoxib, Valdez, Valdixx, Valdy, Valentac, Valoxib, Valtune, Valus AT, Valz, Valzer, Vamid, Vantal, Vantelin, VAP-1 SSAO Inhibitor, vapaliximab, varespladib methyl, Varicosin, Varidase, vascular adhesion protein-1 antibody, VB110, VB120, VB201, VBY285, Vectra-P, vedolizumab, Vefren, VEGFR-1 Antibody, Veldona, veltuzumab, Vendexine, Venimmun N, Venoforte, Venoglobulin-IH, Venozel, Veral, Verax, vercirnon, vero-dexamethasone, Vero-Kladribin, Vetazone, VGX1027, VGX750, Vibex MTX, vidofludimus, Vifenac, Vimovo, Vimultisa, Vincort, Vingraf, Vioform-HC, Vioxl, Vioxx, Virobron, visilizumab, Vivaglobin, Vivalde Plus, Vivian-A, VLST002, VLST003, VLST004, VLST005, VLST007, Voalla, voclosporin, Vokam, Vokmor, Volmax, Volna-K, Voltadol, Voltagesic, Voltanase, Voltanec, Voltaren, Voltarile, Voltic, Voren, vorsetuzumab, Votan-SR, VR909, VRA002, VRP1008, VRS826, VRS826, VT111, VT214, VT224, VT310, VT346, VT362, VTX763, Vurdon, VX30 antibody, VX467, VX5, VX509, VX702, VX740, VX745, VX745, VX850, W54011, Walacort, Walix, WC3027, Wilgraf, Winflam, Winmol, Winpred, Winsolve, Wintogeno, WIP901, Woncox, WSB711 antibody, WSB712 antibody, WSB735, WSB961, X071NAB, X083NAB, Xantomicin Forte, Xedenol, Xefo, Xefocam, Xenar, Xepol, X-Flam, Xibra, Xicam, Xicotil, Xifaxan, XL499, XmAb5483, XmAb5485, XmAb5574, XmAb5871, XOMA052, Xpress, XPro1595, XtendTNF, XToll, Xtra, Xylex-H, Xynofen SR, Yang Shu-IVIG, YHB14112, YM974, Youfeline, Youfenac, Yuma, Yumerol, Yuroben, YY piroxicam, Z104657A, Zacy, Zaltokin, zaltoprofen, Zap70 Inhibitor, Zeepain, Zeloxim Fort, Zema-Pak, Zempack, Zempred, Zenapax, Zenas, Zenol, Zenos, Zenoxone, Zerax, Zerocam, Zerospasm, ZFNs, zinc oxide, Zipsor, ziralimumab, Zitis, Zix-S, Zocort, Zodixam, Zoftadex, zoledronic acid, Zolfin, Zolterol, Zopyrin, Zoralone, ZORprin, Zortress, ZP1848, zucapsaicin, Zunovate, Zwitterionic polysaccharides, ZY1400, Zybodies, Zycel, Zyrofen, Zyrogen Inhibitors, Zyser, Zytrim, and Zywin-Forte. In addition, the anti-inflammatory drugs, as listed above, may be combined with one or more agents listed above or herein or with other agents known in the art.

In one embodiment, the drug is a drug that inhibits, reduces or modulates the signaling and/or activity of PDGF-receptors (PDGFR). For example, in one embodiment, the PDGFR modulator is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFRα or PDGFRβ. In one embodiment, the PDGF antagonist is the anti-PDGF-β aptamer E10030, dasatinib, sunitinib, axitinib, sorefenib, imatinib, imatinib mesylate, nintedanib, pazopanib HCl, ponatinib, MK-2461, pazopanib, crenolanib, PP-121, telatinib, imatinib, KRN 633, CP 673451, TSU-68 (orantinib), Ki8751, amuvatinib, tivozanib, masitinib, motesanib diphosphate, dovitinib, dovitinib dilactic acid, FOVISTA, or linifanib (ABT-869). In a further embodiment, the PDGF antagonist also has VEGF antagonist activity. For example, an anti-VEGF/PDGF-B darpin, dasatinib, dovitinib, Ki8751, telatinib, TSU-68 (orantinib) or motesanib diphosphate are known inhibitors of both VEGF and PDGF, and can be used in the methods described herein.

Examples of other suitable drugs for use with the devices and methods described herein include, but are not limited to: A0003, A36 peptide, AAV2-sFLT01, ACE041, ACU02, ACU3223, ACU4429, AdPEDF, aflibercept, AG13958, aganirsen, AGN150998, AGN745, AL39324, AL78898A, AL8309B, ALN-VEG01, alprostadil, AM1101, amyloid beta antibody, anecortave acetate, Anti-VEGFR-2 Alterase, Aptocine, APX003, ARC1905, ARC1905 with Lucentis, ATG3, ATP-binding cassette, sub-family A, member 4 gene, ATXS10, Avastin with Visudyne, AVT101, AVT2, bertilimumab, bevacizumab with verteporfin, bevasiranib sodium, bevasiranib sodium; with ranibizumab, brimonidine tartrate, BVA301, canakinumab, Cand5, Cand5 with Lucentis, CERE140, ciliary neurotrophic factor, CLT009, CNTO2476, collagen monoclonal antibody, complement component 5 aptamer (pegylated), complement component 5 aptamer (pegylated) with ranibizumab, complement component C3, complement factor B antibody, complement factor D antibody, copper oxide with lutein, vitamin C, vitamin E, and zinc oxide, dalantercept, DE109, bevacizumab, ranibizumab, triamcinolone, triamcinolone acetonide, triamcinolone acetonide with verteporfin, dexamethasone, dexamethasone with ranibizumab and verteporfin, disitertide, DNA damage inducible transcript 4 oligonucleotide, E10030, E10030 with Lucentis, EC400, eculizumab, EGP, EHT204, embryonic stem cells, human stem cells, endoglin monoclonal antibody, EphB4 RTK Inhibitor, EphB4 Soluble Receptor, ESBA1008, ETX6991, Evizon, Eyebar, EyePromise Five, Eyevi, Eylea, F200, FCFD4514S, fenretinide, fluocinolone acetonide, fluocinolone acetonide with ranibizumab, fms-related tyrosine kinase 1 oligonucleotide, fms-related tyrosine kinase 1 oligonucleotide with kinase insert domain receptor 169, fosbretabulin tromethamine, Gamunex, GEM220, GS101, GSK933776, HC31496, Human n-CoDeR, HYB676, IBI-20089 with ranibizumab (Lucentis®), iCo-008, Icon1, I-Gold, Ilaris, Iluvien, Iluvien with Lucentis, immunoglobulins, integrin alpha5beta1 immunoglobulin fragments, Integrin inhibitor, IRIS Lutein, I-Sense Ocushield, Isonep, isopropyl unoprostone, JPE1375, JSM6427, KH902, LentiVue, LFG316, LP590, LPO1010AM, Lucentis, Lucentis with Visudyne, Lutein ekstra, Lutein with myrtillus extract, Lutein with zeaxanthin, M200, M200 with Lucentis, Macugen, MC1101, MCT355, mecamylamine, Microplasmin, motexafin lutetium, MP0112, NADPH oxidase inhibitors, aeterna shark cartilage extract (Arthrovas™, Neoretna™, Psovascar™), neurotrophin 4 gene, Nova21012, Nova21013, NT501, NT503, Nutri-Stulln, ocriplasmin, OcuXan, Oftan Macula, Optrin, ORA102 with bevacizumab (Avastin®), P144, P17, Palomid 529, PAN90806, Panzem, Panzem, PARP inhibitors, pazopanib hydrochloride, pegaptanib sodium, PF4523655, PG11047, piribedil, platelet-derived growth factor beta polypeptide aptamer (pegylated), platelet-derived growth factor beta polypeptide aptamer (pegylated) with ranibizumab, PLG101, PMX20005, PMX53, POT4, PRS055, PTK787, ranibizumab, ranibizumab with triamcinolone acetonide, ranibizumabwith verteporfin, ranibizumab with volociximab, RD27, Rescula, Retaane, retinal pigment epithelial cells, RetinoStat, RG7417, RN6G, RT101, RTU007, SB267268, serpin peptidase inhibitor, clade F, member 1 gene, shark cartilage extract, Shefl, SIR1046, SIR1076, Sirna027, sirolimus, SMTD004, Snelvit, SOD Mimetics, Soliris, sonepcizumab, squalamine lactate, ST602, StarGen, T2TrpRS, TA106, talaporfin sodium, Tauroursodeoxycholic acid, TG100801, TKI, TLCx99, TRC093, TRC105, Trivastal Retard, TT30, Ursa, ursodiol, Vangiolux, VAR10200, vascular endothelial growth factor antibody, vascular endothelial growth factor B, vascular endothelial growth factor kinoid, vascular endothelial growth factor oligonucleotide, VAST Compounds, vatalanib, VEGF antagonist (e.g., as described herein), verteporfin, Visudyne, Visudyne with Lucentis and dexamethasone, Visudyne with triamcinolone acetonide, Vivis, volociximab, Votrient, XV615, zeaxanthin, ZFP TF, zinc-monocysteine and Zybrestat. In one embodiment, one or more of the drugs described above is combined with one or more agents listed above or herein or with other agents known in the art.

In one embodiment, the drug is interferon gamma 1b (Actimmune®) with pirfenidone, ACUHTR028, AlphaVBeta5, aminobenzoate potassium, amyloid P, ANG1122, ANG1170, ANG3062, ANG3281, ANG3298, ANG4011, Anti-CTGF RNAi, Aplidin, Astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, Galectin-3 inhibitors, GKT137831, GMCT01, GMCT02, GRMD01, GRMD02, GRN510, Heberon Alfa R, interferon alfa-2b, interferon gamma-1b with pirfenidone, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 Fusion Proteins, RXI109, secretin, STX100, TGF-beta Inhibitor, transforming growth factor, beta receptor 2 oligonucleotide, VA999260 or XV615. In one embodiment, one or more of the drugs for treating macular edema associated with uveitis described above is combined with one or more agents listed above or herein or with other agents known in the art.

In some embodiments, the drug is AKB9778, bevasiranib sodium, Cand5, choline fenofibrate, Cortiject, c-raf 2-methoxyethyl phosphorothioate oligonucleotide, DE109, dexamethasone, DNA damage inducible transcript 4 oligonucleotide, FOV2304, iCo007, KH902, MP0112, NCX434, Optina, Ozurdex, PF4523655, SAR1118, sirolimus, SK0503 or TriLipix.

In some embodiments, the drug is selected from VEGF modulators, PDGF modulators, anti-inflammatory drugs. Examples of drugs that can be administered via IVT include, but are not limited to: A0003, A0006, Acedolone, AdPEDF, aflibercept, AG13958, aganirsen, AGN208397, AKB9778, AL78898A, amyloid P, Angiogenesis Inhibitor Gene Therapy, ARC1905, Aurocort, bevasiranib sodium, brimonidine, Brimonidine, brimonidine tartrate, bromfenac sodium, Cand5, CERE140, Ciganclor, CLT001, CLT003, CLT004, CLT005, complement component 5 aptamer (pegylated), complement factor D antibody, Cortiject, c-raf 2-methoxyethyl phosphorothioate oligonucleotide, cyclosporine, triamcinolone, DE109, denufosol tetrasodium, dexamethasone, dexamethasone phosphate, disitertide, DNA damage inducible transcript 4 oligonucleotide, E10030, ecallantide, EG3306, Eos013, ESBA1008, ESBA105, Eylea, FCFD4514S, fluocinolone acetonide, fms-related tyrosine kinase 1 oligonucleotide, fomivirsen sodium, fosbretabulin tromethamine, FOV2301, FOV2501, ganciclovir, ganciclovir sodium, GS101, GS156, hyaluronidase, IBI20089, iCo007, Iluvien, INS37217, Isonep, JSM6427, Kalbitor, KH902, lerdelimumab, LFG316, Lucentis®, M200, Macugen, Makyueido, Microplasmin, MK0140, MP0112, NCX434, neurotrophin 4 gene, OC10X, ocriplasmin, ORA102, Ozurdex, P144, P17, Palomid 529, pazopanib hydrochloride, pegaptanib sodium, Plasma Kallikrein Inhibitors, platelet-derived growth factor beta polypeptide aptamer (pegylated), POT4, PRM167, PRS055, QPI1007, ranibizumab, resveratrol, Retilone, retinal pigment epithelium-specific protein 65 kDa gene, Retisert, rod derived cone viability factor, RPE65 Gene Therapy, RPGR Gene Therapy, RTP801, Sd-rxRNA, serpin peptidase inhibitor clade F member 1 gene, Sirna027, sirolimus, sonepcizumab, SRT501, STP601, TG100948, Trabio, triamcinolone, triamcinolone acetonide, Trivaris, tumor necrosis factor antibody, VEGF/rGel-Op, verteporfin, Visudyne, Vitrase, Vitrasert, Vitravene, Vitreals, volociximab, Votrient, XG102, Xibrom, XV615, and Zybrestat

While various embodiments have been described above, it should be understood that they have been presented by way of example only, and not limitation. While the embodiments have been particularly shown and described, it will be understood that various changes in form and details may be made. Where schematics and/or embodiments described above indicate certain components arranged in certain orientations or positions, the arrangement of components may be modified. Although various embodiments have been described as having particular features and/or combinations of components, other embodiments are possible having a combination of any features and/or components from any of embodiments as discussed above.

1. A method, comprising:
measuring a physical property of an eye of a subject;
increasing a permeability of the eye of the subject; and
after the increasing the permeability of the eye, producing a stream of medicament directed towards a suprachoroidal space of the eye of the subject based on the measured physical property.

2. The method of claim 1, wherein the measuring includes emitting an infrared excitation signal or an ultrasonic excitation sound wave towards the eye and sensing a response to the excitation signal or the excitation sound wave.

3. The method of claim 1, wherein the physical property includes at least one of a thickness of a scleral layer of the eye, a thickness of a choroid layer of the eye, or a thickness of a retinal of the eye.

4. The method of claim 1, wherein the physical property includes at least one of a permeability of a scleral layer, a permeability of a chordoid layer of the eye, or a permeability of a retinal layer of the eye.

5. The method of claim 1, wherein the measuring includes measuring a distance from an outer surface of the eye to an interface between a scleral layer of the eye and a choroid layer of the eye.

6. The method of claim 1, wherein the increasing the permeability of the eye includes delivering a chemical species to increase permeability of a scleral layer of the eye.

7. The method of claim 1, wherein the increasing the permeability of the eye includes delivering an ultrasound perturbation at a frequency having sufficient intensity to reduce resistance of a sclera of the eye.

8. The method of claim 1, wherein the increasing the permeability of the eye includes delivering thermal ablation to a sclera of the eye.

9. The method of claim 8, wherein the delivering the thermal ablation includes delivering optical energy using a laser gun, causing evaporation of water and formation of microchannels within the sclera to facilitate diffusion of the stream of medicament.

10. The method of claim 8, wherein prior to or during the delivering thermal ablation, disrupting a conjunctiva layer of the eye to expose a surface of the sclera, the delivering the optical energy including delivering the optical energy directly to the sclera.

11. The method of claim 8, wherein the delivering the thermal ablation includes adjusting at least one of wavelength, a frequency, a pulse length, a pulse energy, a number of pulses, an exposure time, or pulse repetition during the delivering.

12. The method of claim 11, wherein the adjusting is based on the measured physical property.

13. The method of claim 1, wherein the increasing the permeability of the eye includes disposing one or more electrodes on an external surface of the eye, and passing an excitation direct current on the eye.

14. The method of claim 1, wherein the suprachoroidal space of the eye is located at an anterior portion of the eye.

15. An apparatus, comprising:
a housing;
a medicament container configured to accommodate a medicinal component;
a sensor configured to measure a physical property of an eye of a subject; and
an injector coupled to the medicament container and configured to produce a stream of the medicinal component directed towards a region of the eye of the subject based on the measured physical property.

16. The apparatus of claim 15, wherein:
the injector includes a high-pressure chamber comprising:
a compressed gas; and
a piston, the piston being coupled to the medicament container;
the compressed gas is configured to expand from an initial pressure to move the piston and generate the stream of the medicinal component; and
the initial pressure is determined based on the measured physical property of the eye of the subject.

17. The apparatus of claim 16 further comprising a processor operatively coupled to the injector and the sensor, the processor being configured to:
receive a signal from the sensor indicative of the physical property of the eye;
determine the physical property of the eye based on the received signal; and
determine the initial pressure of the high-pressure chamber.

18. The apparatus of claim 15, wherein:
the injector includes a Lorentz motor comprising:
a coil; and
a piston, the piston being coupled to the medicament container;
the coil is configured to pass an electric current to generate a magnetic force to move the piston and generate the stream of the medicinal component; and
the electric current is determined based on the measured physical property of the eye of the subject.

19. The apparatus of claim 18, further comprising a processor operatively coupled to the injector and the sensor, the processor being configured to:
receive a signal from the sensor indicative of the physical property of the eye;
determine the physical property of the eye based on the received signal; and
determine the electric current of the Lorentz motor.

20. The apparatus of claim 15, wherein:
the injector includes a Lorentz actuator comprising a coil;
the coil is configured to pass an electric current to generate a magnetic force to move the medicinal components and generate the stream of the medicinal component; and
the electric current is determined based on the measured physical property of the eye of the subject.

21. The apparatus of claim 19, further comprising a processor operatively coupled to the injector and the sensor, the processor being configured to:
receive a signal from the sensor indicative of the physical property of the eye;
determine the physical property of the eye based on the received signal; and
determine the electric current of the Lorentz actuator.

22. The apparatus of claim 15, further comprising a tissue manipulator, the tissue manipulator being configured to increase a permeability of the eye of the subject.

23. The apparatus of claim 22, wherein the tissue manipulator includes a thermal ablation device configured to heat a small area of an ocular tissue layer of the eye of the subject.

24. The apparatus of claim 23, wherein the thermal ablation device includes a laser gun.

25. The apparatus of claim 22, wherein the tissue manipulator includes:
a reservoir containing a chemical species, the chemical species configured to increase the permeability of a sclera layer of the eye of the subject; and
a chemical reagent injector fluidically coupled to the reservoir and configured to deliver the chemical species to the sclera od the eye of the subject.

26. The apparatus of claim 22, wherein the tissue manipulator includes an ultrasound actuator, the ultrasound actuator configured to deliver an ultrasound perturbation at a frequency having sufficient intensity to reduce resistance of a sclera of the eye.
